(19) United States
(12) Patent Application Publication
(10) Pub. No.: US 2006/0294608 A1
(43) Pub. Date: Dec. 28, 2006

(54) Novel haemopoietin receptor and genetic sequences encoding same

(75) Inventors: Douglas J. Hilton, Warrandyte (AU); Nicos A. Nicola, Mont Albert (AU); Alison Farley, Port Fairy (AU); Tracy Willson, North Balwyn (AU); Jian-Guo Zhang, North Melbourne (AU); Warren Alexander, Moonee Ponds (AU); Steven Rakar, Avondale Heights (AU); Louis Fabri, Diamond Creek (AU); Tetsuo Kojima, Gotemba (JP); Masatsugu Maeda, Tsukuba (JP); Yasufumi Kikuchi, Mishima (JP); Andrew Nash, Kew (AU)

Correspondence Address: SCULLY SCOTT MURPHY & PRESSER, PC, 400 GARDEN CITY PLAZA, SUITE 300, GARDEN CITY, NY, 11530, US

(73) Assignee: Zenyth Operations Pty Ltd, Richmond (AU)

(21) Appl. No.: 11/509,410

(22) Filed: Aug. 24, 2006

Related U.S. Application Data
(63) Continuation of application No. 09/037,657, filed on Mar. 10, 1998.
Application No. 09/037,657 is a continuation-in-part of application No. 08/928,720, filed on Sep. 11, 1997, now abandoned.

(30) Foreign Application Priority Data
Sep. 11, 1996 (AU) PO 2246/96

(51) Int. Cl. A01K 67/027; C07H 21/04; C12P 21/06; C07K 14/71; C07K 16/28

(52) U.S. Cl. 800/14; 800/18; 530/350; 530/388.22; 435/69.1; 435/320.1; 435/325; 514/12; 536/23.5

(57) ABSTRACT

The present invention relates generally to a novel haemopoietin receptor or derivatives thereof and to genetic sequences encoding same. Interaction between the novel receptor of the present invention and a cytokine ligand facilitates proliferation, differentiation and survival of a wide variety of cells. The novel receptor and its derivatives and the genetic sequences encoding same of the present invention are useful in the development of a wide range of agonists, antagonists, therapeutics and diagnostic reagents based on ligand interaction with its receptor.

### CROSS REFERENCE TO RELATED APPLICATIONS

This application is a continuation of U.S. patent application Ser. No. 09/037,657, filed Mar. 10, 1998, which is a continuation-in-part of U.S. patent application Ser. No. 08/928,720 filed Sep. 11, 1997 now abandoned.

### FIELD OF THE INVENTION

The present invention relates generally to a novel haemopoietin receptor or derivatives thereof and to genetic sequences encoding same. Interaction between the novel receptor of the present invention and a ligand facilitates proliferation, differentiation and survival of a wide variety of cells. The novel receptor and its derivatives and the genetic sequences encoding same of the present invention are useful in the development of a wide range of agonists, antagonists, therapeutics and diagnostic reagents based on ligand interaction with its receptor.

Bibliographic details of the publications numerically referred to in this specification are collected at the end of the description. Sequence Identity Numbers (SEQ ID NOs.) for the nucleotide and amino acid sequences referred to in the specification are defined following the bibliography.

### BACKGROUND OF THE INVENTION

Throughout this specification and the claims which follow, unless the context requires otherwise, the word “comprise”, or variations such as “comprises” or “comprising”, will be understood to imply the inclusion of a stated integer or group of integers but not the exclusion of any other integer or group of integers.

The rapidly increasing sophistication of recombinant DNA techniques is greatly facilitating research into the medical and allied health fields. Cytokine research is of particular importance, especially as these molecules regulate the proliferation, differentiation and function of a wide variety of cells. Administration of recombinant cytokines or regulating cytokine function and/or synthesis is becoming increasingly the focus of medical research into the treatment of a range of disease conditions.

Despite the discovery of a range of cytokines and other secreted regulators of cell function, comparatively few cytokines are directly used or targeted in therapeutic regimens. One reason for this is the pleiotropic nature of many cytokines. For example, interleukin (IL)-1 is a functionally pleiotropic molecule (1,2), initially characterized by its ability to stimulate proliferation of the IL-6-dependent plasmacytoma cell line, T11 65 (3). Other biological actions of IL-11 include induction of multipotential haemopoietin progenitor cell proliferation (4,5,6), enhancement of megakaryocyte and platelet formation (7,8,9,10), stimulation of acute phase protein synthesis (11) and inhibition of adipocyte lipoprotein lipase activity (12, 13).

Other important cytokines in the IL-11 group include IL-6, leukaemia inhibitory factor (LIF), oncostatin M (OSM) and CNTF. All these cytokines exhibit pleiotropic properties with significant activities in proliferation, differentiation and survival of cells. Members of the haemopoietin receptor family are defined by the presence of a conserved amino acid domain in their extracellular region. However, despite the low level of amino acid sequence conservation between other haemopoietin receptor domains of different receptors, they are all predicted to assume a similar tertiary structure, centred around two fibronectin-type III repeats (18,19).

The size of the haemopoietin receptor family has now become extensive and includes the cell surface receptors for may cytokines including interleukin-2 (IL-2), IL-3, IL-4, IL-5, IL-6, IL-7, IL-9, IL-11, IL-12, IL-13, IL-15, granulocyte colony stimulating factor (G-CSF), granulocyte-macrophage-CSF (GM-CSF), erythropoietin, thrombopoietin, leptin, leukaemia inhibitory factor, oncostatin-M, ciliary neurotrophic factor, cardiotrophin, growth hormone and prolactin. Although most of the members of the haemopoietin receptor family act as classic cell surface receptors, binding their cognate ligand at the cell surface and initiating intracellular signal transduction, some receptors are also produced in naturally occurring soluble forms. These soluble receptors can either act as cytokine antagonists, by binding to cytokines and inhibiting productive interactions with cell surface receptors (eg LIF binding protein; (20) or as agonists, binding to cytokine and potentiating interaction with cell surface receptor components (eg soluble interleukin-6 receptor a-chain; (21). Still other members of the family appear to be produced only as secreted proteins, with no evidence of a cell surface form. In this regard, the IL-12 p40 subunit is a useful example. The cytokine IL-12 is secreted as a heterodimer composed of a p35 subunit which shows similarity to cytokines such as IL-6 (22) and a p40 subunit which shares similarity with the IL-6 receptor a-chain (23). In this case the soluble receptor acts as part of the cytokine itself and essential to formation of an active protein. In addition to acting as cytokines (eg IL-12p40), cytokine agonists (eg IL-6 receptor a-chain) or cytokine antagonists (LIF binding protein), members of the haemopoietin receptor have been useful in the discovery of small molecule cytokine mimetics. For example, the discovery of peptide mimetics of two commercially valuable cytokines, erythropoietin and thrombopoietin, centred on the selection of peptides capable of binding to soluble versions of the erythropoietin and thrombopoietin receptors (24,25). Due to the importance and multifactorial nature of these cytokines, there is a need to identify receptors, including both cell bound and soluble, for pleiotropic cytokines. Identification of such receptors permits the identification of pleiotropic cytokines and the development of a range of therapeutic and diagnostic agents.

### DETAILED DESCRIPTION OF THE INVENTION

One aspect of the present invention relates to a nucleic acid molecule comprising a sequence of nucleotides encoding or complementary to a sequence encoding a novel haemopoietin receptor or a derivative thereof.

More particularly, the present invention provides a nucleic acid molecule comprising a sequence of nucleotides encoding or complementary to a sequence encoding a novel haemopoietin receptor or a derivative thereof having the motif:

| | |
|---|---|
| Trp Ser Xaa Trp Ser, | [SEQ ID NO: 1] |

wherein Xaa is any amino acid and is preferably Asp or Glu.

Even more particularly, the present invention is directed to a nucleic acid molecule comprising a sequence of nucleotides encoding or complementary to a sequence encoding a novel haemopoietin receptor or a derivative thereof, said receptor comprising the motif:

| | |
|---|---|
| Trp Ser Xaa Trp Ser | [SEQ ID NO: 1] |

wherein Xaa is any amino acid and is preferably Asp or Glu, said nucleic acid molecule is identifiable by hybridisation to said molecule under low stringency conditions at 42° C. with

| | |
|---|---|
| 5′ (A/G)CTCCA(A/G)TC(A/G)CTCCA 3′ | [SEQ ID NO: 7] |
| and | |
| 5′ (A/G)CTCCA(C/T)TC(A/G)CTCCA 3′. | [SEQ ID NO: 8] |

Still more particularly, the present invention provides an isolated nucleic acid molecule comprising a sequence of nucleotides substantially as set forth in SEQ ID NO:12 or a nucleotide sequence having at least 60% similarity to the nucleotide sequence set forth in SEQ ID NO:12 or a nucleotide sequence capable of hybridising thereto under low stringency conditions at 42° C. and wherein said nucleotide sequence encodes a novel haemopoietin receptor or a derivative thereof.

In a related embodiment, the present invention provides an isolated nucleic acid molecule comprising a sequence of nucleotides substantially as set forth in SEQ ID NO:14 or a nucleotide sequence having at least 60% similarity to the nucleotide sequence set forth in SEQ ID NO:14 or a nucleotide sequence capable of hybridising thereto under low stringency conditions at 42° C. and wherein said nucleotide sequence encodes a novel haemopoietin receptor or a derivative thereof.

In another related embodiment, the present invention provides an isolated nucleic acid molecule comprising a sequence of nucleotides substantially as set forth in SEQ ID NO:16 or a nucleotide sequence having at least 60% similarity to the nucleotide sequence set forth in SEQ ID NO:16 or a nucleotide sequence capable of hybridising thereto under low stringency conditions at 42° C. and wherein said nucleotide sequence encodes a novel haemopoietin receptor or a derivative thereof.

In a further related embodiment, the present invention provides an isolated nucleic acid molecule comprising a sequence of nucleotides substantially as set forth in SEQ ID NO:18 or a nucleotide sequence having at least 60% similarity to the nucleotide sequence set forth in SEQ ID NO:18 or a nucleotide sequence capable of hybridising thereto under low stringency conditions at 42° C. and wherein said nucleotide sequence encodes a novel haemopoietin receptor or a derivative thereof.

In yet a further related embodiment, the present invention provides an isolated nucleic acid molecule comprising a sequence of nucleotides substantially as set forth in SEQ ID NO:24 or a nucleotide sequence having at least 60% similarity to the nucleotide sequence set forth in SEQ ID NO:24 or a nucleotide sequence capable of hybridising thereto under low stringency conditions at 42° C. and wherein said nucleotide sequence encodes a novel haemopoietin receptor or a derivative thereof.

Still yet a further embodiment of the present invention is directed to a sequence of nucleotides substantially as set forth in SEQ ID NO:28 or a nucleotide sequence having at least 60% similarity to the nucleotide sequence set forth in SEQ ID NO:28 or a nucleotide sequence capable of hybridising thereto under low stringency conditions at 42° C. and wherein said nucleotide sequence encodes a novel haemopoietin receptor or a derivative thereof.

In still yet another embodiment, the present invention provides an isolated nucleic acid molecule comprising a sequence of nucleotides substantially set forth in SEQ ID NO:38 or a nucleotide sequence having at least 60% similarity to the nucleotide sequence set forth in SEQ ID NO:38 or a nucleotide sequence capable of hybridising thereto under low stringency conditions at 42° C. and wherein said nucleotide sequence encodes a novel haemopoietin receptor or a derivative thereof.

Another embodiment of the present invention provides an isolated nucleic acid molecule comprising a sequence of nucleotides substantially set forth in SEQ ID NO:43 or a nucleotide sequence having at least 60% similarity to the nucleotide sequence set forth in SEQ ID NO:43 or a nucleotide sequence capable of hybridising thereto under low stringency conditions at 42° C. and wherein said nucleotide sequence encodes a novel haemopoietin receptor or a derivative thereof.

The term “receptor” is used in its broadest sense and includes any molecule capable of binding, associating or otherwise interacting with a ligand. Generally, the interaction will have a signalling effect although the present invention is not necessarily so limited. For example, the “receptor” may be in soluble form, often referred to as a cytokine binding protein. A receptor may be deemed a receptor notwithstanding that its ligand or ligands has or have not been identified.

Preferably, the novel receptor is derived from a mammal or a species of bird. Particularly, preferred mammals include humans, primates, laboratory test animals (e.g. mice, rats, rabbits, guinea pigs), livestock animals (e.g. sheep, horses, pigs, cows), companion animals (e.g. dogs, cats) or captive wild animals (e.g. deer, foxes, kangaroos). Although the present invention is exemplified with respect to mice, the scope of the subject invention extends to all animals and in particular humans.

The present invention is predicated in part on an ability to identify members of the haemopoietin receptor family with limited sequence similarity. Based on this approach, a genetic sequence has been identified in accordance with the present invention which encodes a novel receptor. The expressed genetic sequence is referred to herein as “NR6”. Different forms of NR6 are referred to as, for example, NR6.1, NR6.2 and NR6.3. The nucleotide and corresponding amino acid sequences for these molecules are represented in SEQ ID NOs: 12, 14 and 16, respectively.

Preferred human and murine nucleic acid sequences for NR6 or its derivatives include sequences from brain, liver, kidney, neonatal, embryonic, cancer or tumour-derived tissues.

Reference herein to a low stringency at 42° C. includes and encompasses from at least about 1% v/v to at least about 15% v/v formamide and from at least about 1M to at least about 2M salt for hybridisation, and at least about 1M to at least about 2M salt for washing conditions. Alternative stringency conditions may be applied where necessary, such as medium stringency, which includes and encompasses from at least about 16% v/v to at least about 30% v/v formamide and from at least about 0.5M to at least about 0.9M salt for hybridisation, and at least about 0.5M to at least about 0.9M salt for washing conditions, or high stringency, which includes and encompasses from at least about 31% v/v to at least about 50% v/v formamide and from at least about 0.01M to at least about 0.15M salt for hybridisation, and at least about 0.01M to at least about 0.15M salt for washing conditions.

The nucleic acid molecules contemplated by the present invention are generally in isolated form and are preferably cDNA or genomic DNA molecules. In a particularly preferred embodiment, the nucleic acid molecules are in vectors and most preferably expression vectors to enable expression in a suitable host cell. Particularly useful host cells include prokaryotic cells, mammalian cells, yeast cells and insect cells. The cells may also be in the form of a cell line.

Accordingly, another aspect of the present invention provides an expression vector comprising a nucleic acid molecule encoding the novel haempoietin receptor or a derivative thereof as hereinbefore described, said expression vector capable of expression in a selected host cell.

Another aspect of the present invention contemplates a method for cloning a nucleotide sequence encoding NR6 or a derivative thereof, said method comprising searching a nucleotide data base for a sequence which encodes the amino acid sequence set forth in SEQ ID NO:1, designing one or more oligonucleotide primers based on the nucleotide sequence located in the search, screening a nucleic acid library with said one or more oligonucleotides and obtaining a clone therefrom which encodes said NR6 or part thereof.

Once a novel nucleotide sequence is obtained as indicated above encoding NR6, oligonucleotides may be designed which bind cDNA clones with high stringency. Direct colony hybridisation may be employed or PCR amplification may be used. The use of oligonucleotide primers which bind under conditions of high stringency ensures rapid cloning of a molecule encoding the novel NR6 and less time is required in screening out cloning artefacts. However, depending on the primers used, low or medium stringency conditions may also be employed.

Alternatively, a library may be screened directly such as using oligonucleotides set forth in SEQ ID NO:7 or SEQ ID NO:8 or a mixture of both oligonucleotides may be used. In addition, one or more of oligonucleotides defined in SEQ ID NO:2 to 11 may also be used.

Preferably, the nucleic acid library is a cDNA, genomic, cDNA expression or mRNA library.

Preferably, the nucleic acid library is a cDNA expression library. Preferably, the nucleotide data base is of human or murine origin and of brain, liver, kidney, neo-natal tissue, embryonic tissue, tumour or cancer tissue origin.

Preferred percentage similarities to the reference nucleotide sequences include at least about 70%, more preferably at least about 80%, still more preferably at least about 90% and even more preferably at least about 95% or above.

Another aspect of the present invention provides an isolated nucleic acid molecule comprising a sequence of nucleotides encoding a novel haempoietin receptor or derivative thereof having an amino acid sequence as set forth in SEQ ID NO:13 or having at least about 50% similarity to all or part thereof.

Still yet another aspect of the present invention provides an isolated nucleic acid molecule comprising a sequence of nucleotides encoding a novel haempoietin receptor or derivative thereof having an amino acid sequence as set forth in SEQ ID NO:15 or having at least about 50% similarity to all or part thereof.

Even yet another aspect of the present invention provides an isolated nucleic acid molecule comprising a sequence of nucleotides encoding a novel haempoietin receptor or derivative thereof having an amino acid sequence as set forth in SEQ ID NO:17 or having at least about 50% similarity to all or part thereof.

A further aspect of the present invention provides an isolated nucleic acid molecule comprising a sequence of nucleotides encoding a novel haempoietin receptor or derivative thereof having an amino acid sequence as set forth in SEQ ID NO:19 or having at least about 50% similarity to all or part thereof.

Even yet a another aspect of the present invention provides an isolated nucleic acid molecule comprising a sequence of nucleotides encoding a novel haempoietin receptor or derivative thereof having an amino acid sequence as set forth in SEQ ID NO:25 or having at least about 50% similarity to all or part thereof.

Another aspect of the present invention provides an isolated nucleic acid molecule comprising a sequence of nucleotides encoding a novel haempoietin receptor or derivative thereof having an amino acid sequence as set forth in one or more of SEQ ID NOs:29 or having at least about 50% similarity to all or part thereof.

Still another aspect of the present invention provides an isolated nucleic acid molecule comprising a sequence of nucleotides encoding a novel haemopoietin receptor or derivative thereof having an amino acid sequence as set forth in one or more of SEQ ID NOs:44 or having at least about 50% similarity to all or part thereof.

Preferably, the percentage amino acid similarity is at least about 60%, more preferably at least about 70%, even more preferably at least about 80-85% and still even more preferably at least about 90-95% or greater.

The NR6 polypeptide contemplated by the present invention includes, therefore, derivatives which are components, parts, fragments, homologues or analogues of the novel haempoietin receptors which are preferably encoded by all or part of a nucleotide sequences substantially set forth in SEQ ID NO:12 or 14 or 16 or 18 or 25 or 20 or 24 or 28 or 38 or 43 or a molecule having at least about 60% nucleotide similarity to all or part thereof or a molecule capable of hybridising to the nucleotide sequence set forth in SEQ ID NO:12 or 14 or 16 or 18 or 20 or 24 or 28 or 38 or 43 or a complementary form thereof. The NR6 molecule may be glycosylated or non-glycosylated. When in glycosylated form, the glycosylation may be substantially the same as naturally occurring haempoietin receptor or may be a modified form of glycosylation. Altered or differential glycosylation states may or may not affect binding activity of the novel receptor.

The NR6 haemopoietin receptor may be in soluble form or may be expressed on a cell surface or conjugated or fused to a solid support or another molecule.

As stated above, the present invention further contemplates a range of derivatives of NR6. Derivatives include fragments, parts, portions, mutants, homologues and analogues of the NR6 polypeptide and corresponding genetic sequence. Derivatives also include single or multiple amino acid substitutions, deletions and/or additions to NR6 or single or multiple nucleotide substitutions, deletions and/or additions to the genetic sequence encoding NR6. “Additions” to amino acid sequences or nucleotide sequences include fusions with other peptides, polypeptides or proteins or fusions to nucleotide sequences. Reference herein to “NR6” includes reference to all derivatives thereof including functional derivatives or NR6 immunologically interactive derivatives.

Analogues of NR6 contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecule or their analogues.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acid, contemplated herein is shown in Table 1.

These types of modifications may be important to stabilise NR6 if administered to an individual or for use as a diagnostic reagent.

Crosslinkers can be used, for example, to stabilise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids, introduction of double bonds between C_{α}, and C_{β} atoms of amino acids and the formation of cyclic peptides or analogues by introducing covalent bonds such as forming an amide bond between the N and C termini, between two side chains or between a side chain and the N or C terminus.

TABLE 1

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | | |
| ethylamino)cyclopropane | | | |

The present invention further contemplates chemical analogues of NR6 capable of acting as antagonists or agonists of NR6 or which can act as functional analogues of NR6. Chemical analogues may not necessarily be derived from NR6 but may share certain conformational similarities. Alternatively, chemical analogues may be specifically designed to mimic certain physiochemical properties of NR6. Chemical analogues may be chemically synthesised or may be detected following, for example, natural product screening.

The identification of NR6 permits the generation of a range of therapeutic molecules capable of modulating expression of NR6 or modulating the activity of NR6. Modulators contemplated by the present invention includes agonists and antagonists of NR6 expression. Antagonists of NR6 expression include antisense molecules, ribozymes and co-suppression molecules. Agonists include molecules which increase promoter ability or interfere with negative regulatory mechanisms. Agonists of NR6 include molecules which overcome any negative regulatory mechanism. Antagonists of NR6 include antibodies and inhibitor peptide fragments.

Other derivatives contemplated by the present invention include a range of glycosylation variants from a completely unglycosylated molecule to a modified glycosylated molecule. Altered glycosylation patterns may result from expression of recombinant molecules in different host cells.

Another embodiment of the present invention contemplates a method for modulating expression of NR6 in a subject such as a human or mouse, said method comprising contacting the genetic sequence encoding NR6 with an effective amount of a modulator of NR6 expression for a time and under conditions sufficient to up-regulate or down-regulate or otherwise modulate expression of NR6. Modulating NR6 expression provides a means of modulating NR6-ligand interaction or NR6 stimulation of cell activities.

Another aspect of the present invention contemplates a method of modulating activity of NR6 in a human, said method comprising administering to said mammal a modulating effective amount of a molecule for a time and under conditions sufficient to increase or decrease NR6 activity. The molecule may be a proteinaceous molecule or a chemical entity and may also be a derivative of NR6 or its ligand or a chemical analogue or truncation mutant of NR6 or its ligand.

The present invention, therefore, contemplates a pharmaceutical composition comprising NR6 or a derivative thereof or a modulator of NR6 expression or NR6 activity and one or more pharmaceutically acceptable carriers and/or diluents. These components are referred to as the “active ingredients”.

The pharmaceutical forms suitable for injectable use include sterile aqueous solutions (where water soluble) and sterile powders for the extemporaneous preparation of sterile injectable solutions. It must be stable under the conditions of manufacture and storage and must be preserved against the contaminating action of microorganisms such as bacteria and fungi. The carrier can be a solvent or dilution medium comprising, for example, water, ethanol, polyol (for example, glycerol, propylene glycol and liquid polyethylene glycol, and the like), suitable mixtures thereof, and vegetable oils. The proper fluidity can be maintained, for example, by the use of superfactants. The preventions of the action of microorganisms can be brought about by various antibacterial and antifungal agents, for example, parabens, chlorobutanol, phenol, sorbic acid, thirmerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 ug and 2000 mg of active compound. Alternative dosage amounts include from about 1 μg to about 1000 mg and from about 10 μg to about 500 mg.

The tablets, troches, pills, capsules and the like may also contain the components as listed hereafter: A binder such as gum, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The present invention also extends to forms suitable for topical application such as creams, lotions and gels as well as a range of “paints” which are applied to skin and through which the active ingredients are absorbed.

Pharmaceutically acceptable carriers and/or diluents include any and all solvents, dispersion media, coatings, antibacterial and antifungal agents, isotonic and absorption delaying agents and the like. The use of such media and agents for pharmaceutical active substances is well known in the art and except insofar as any conventional media or agent is incompatible with the active ingredient, their use in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired as herein disclosed in detail.

The principal active ingredient is compounded for convenient and effective administration in effective amounts with a suitable pharmaceutically acceptable carrier in dosage unit form as hereinbefore disclosed. A unit dosage form can, for example, contain the principal active compound in amounts ranging from 0.5 μg to about 2000 mg. Expressed in proportions, the active compound is generally present in from about 0.5 μg to about 2000 mg/ml of carrier. In the case of compositions containing supplementary active ingredients, the dosages are determined by reference to the usual dose and manner of administration of the said ingredients.

Dosages may also be expressed per body weight of the recipient. For example, from about 10 ng to about 1000 mg/kg body weight, from about 100 ng to about 500 mg/kg body weight and for about 1 μg to above 250 mg/kg body weight may be administered.

The pharmaceutical composition may also comprise genetic molecules such as a vector capable of transfecting target cells where the vector carries a nucleic acid molecule capable of modulating NR6 expression or NR6 activity. The vector may, for example, be a viral vector.

Still another aspect of the present invention is directed to antibodies to NR6 and its derivatives. Such antibodies may be monoclonal or polyclonal and may be selected from naturally occurring antibodies to NR6 or may be specifically raised to NR6 or derivatives thereof. In the case of the latter, NR6 or its derivatives may first need to be associated with a carrier molecule. The antibodies and/or recombinant NR6 or its derivatives of the present invention are particularly useful as therapeutic or diagnostic agents. For example, NR6 antibodies or antibodies to its ligand may act as antagonists.

For example, NR6 and its derivatives can be used to screen for naturally occurring antibodies to NR6. These may occur, for example in some autoimmune diseases. Alternatively, specific antibodies can be used to screen for NR6. Techniques for such assays are well known in the art and include, for example, sandwich assays and ELISA. Knowledge of NR6 levels may be important for diagnosis of certain cancers or a predisposition to cancers or for monitoring certain therapeutic protocols.

Antibodies to NR6 of the present invention may be monoclonal or polyclonal. Alternatively, fragments of antibodies may be used such as Fab fragments. Furthermore, the present invention extends to recombinant and synthetic antibodies and to antibody hybrids. A “synthetic antibody” is considered herein to include fragments and hybrids of antibodies. The antibodies of this aspect of the present invention are particularly useful for immunotherapy and may also be used as a diagnostic tool for assessing apoptosis or monitoring the program of a therapeutic regimen.

For example, specific antibodies can be used to screen for NR6 proteins. The latter would be important, for example, as a means for screening for levels of NR6 in a cell extract or other biological fluid or purifying NR6 made by recombinant means from culture supernatant fluid. Techniques for the assays contemplated herein are known in the art and include, for example, sandwich assays and ELISA.

It is within the scope of this invention to include any second antibodies (monoclonal, polyclonal or fragments of antibodies or synthetic antibodies) directed to the first mentioned antibodies discussed above. Both the first and second antibodies may be used in detection assays or a first antibody may be used with a commercially available anti-immunoglobulin antibody. An antibody as contemplated herein includes any antibody specific to any region of NR6.

Both polyclonal and monoclonal antibodies are obtainable by immunization with the enzyme or protein and either type is utilizable for immunoassays. The methods of obtaining both types of sera are well known in the art. Polyclonal sera are less preferred but are relatively easily prepared by injection of a suitable laboratory animal with an effective amount of NR6, or antigenic parts thereof, collecting serum from the animal, and isolating specific sera by any of the known immunoadsorbent techniques. Although antibodies produced by this method are utilizable in virtually any type of immunoassay, they are generally less favoured because of the potential heterogeneity of the product.

The use of monoclonal antibodies in an immunoassay is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art.

Another aspect of the present invention contemplates a method for detecting NR6 in a biological sample from a subject said method comprising contacting said biological sample with an antibody specific for NR6 or its derivatives or homologues for a time and under conditions sufficient for an antibody-NR6 complex to form, and then detecting said complex.

The presence of NR6 may be accomplished in a number of ways such as by Western blotting and ELISA procedures. A wide range of immunoassay techniques are available as can be seen by reference to U.S. Pat. Nos. 4,016,043, 4,424,279 and 4,018,653. These, of course, includes both single-site and two-site or “sandwich” assays of the non-competitive types, as well as in the traditional competitive binding assays. These assays also include direct binding of a labelled antibody to a target.

Sandwich assays are among the most useful and commonly used assays and are favoured for use in the present invention. A number of variations of the sandwich assay technique exist, and all are intended to be encompassed by the present invention. Briefly, in a typical forward assay, an unlabelled antibody is immobilized on a solid substrate and the sample to be tested brought into contact with the bound molecule. After a suitable period of incubation, for a period of time sufficient to allow formation of an antibody-antigen complex, a second antibody specific to the antigen, labelled with a reporter molecule capable of producing a detectable signal is then added and incubated, allowing time sufficient for the formation of another complex of antibody-antigen-labelled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of hapten. Variations on the forward assay include a simultaneous assay, in which both sample and labelled antibody are added simultaneously to the bound antibody. These techniques are well known to those skilled in the art, including any minor variations as will be readily apparent. In accordance with the present invention, the sample is one which might contain NR6 including cell extract, tissue biopsy or possibly serum, saliva, mucosal secretions, lymph, tissue fluid and respiratory fluid. The sample is, therefore, generally a biological sample comprising biological fluid but also extends to fermentation fluid and supernatant fluid such as from a cell culture.

In the typical forward sandwich assay, a first antibody having specificity for the NR6 or antigenic parts thereof, is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes or overnight if more convenient) and under suitable conditions (e.g. from about room temperature to about 37° C.) to allow binding of any subunit present in the antibody. Following the incubation period, the antibody subunit solid phase is washed and dried and incubated with a second antibody specific for a portion of the hapten. The second antibody is linked to a reporter molecule which is used to indicate the binding of the second antibody to the hapten.

An alternative method involves immobilizing the target molecules in the biological sample and then exposing the immobilized target to specific antibody which may or may not be labelled with a reporter molecule. Depending on the amount of target and the strength of the reporter molecule signal, a bound target may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.

In another alternative method, the NR6 ligand is immobilised to a solid support and a biological sample containing NR6 brought into contact with its immobilised ligand. Binding between NR5 and its ligand can then be determined using an antibody to NR6 which itself may be labelled with a reporter molecule or a further anti-immunoglobulin antibody labelled with a reporter molecule could be used to detect antibody bound to NR6.

By “reporter molecule” as used in the present specification, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase and alkaline phosphatase, amongst others. The substrates to be used with the specific enzymes are generally chosen for the production, upon hydrolysis by the corresponding enzyme, of a detectable colour change. Examples of suitable enzymes include alkaline phosphatase and peroxidase. It is also possible to employ fluorogenic substrates, which yield a fluorescent product rather than the chromogenic substrates noted above. In all cases, the enzyme-labelled antibody is added to the first antibody hapten complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of hapten which was present in the sample. “Reporter molecule” also extends to use of cell agglutination or inhibition of agglutination such as red blood cells on latex beads, and the like.

Alternately, fluorescent compounds, such as fluorescein and rhodamine, may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic colour visually detectable with a light microscope. As in the EIA, the fluorescent labelled antibody is allowed to bind to the first antibody-hapten complex. After washing off the unbound reagent, the remaining tertiary complex is then exposed to the light of the appropriate wavelength the fluorescence observed indicates the presence of the hapten of interest. Immunofluorescene and EIA techniques are both very well established in the art and are particularly preferred for the present method. However, other reporter molecules, such as radioisotope, chemiluminescent or bioluminescent molecules, may also be employed.

The present invention also contemplates genetic assays such as involving PCR analysis to detect the NR6 gene or its derivatives. Alternative methods or methods used in conjunction include direct nucleotide sequencing or mutation scanning such as single stranded conformational polymorphisms analysis (SSCP) as specific oligonucleotide hybridisation, as methods such as direct protein truncation tests.

The nucleic acid molecules of the present invention may be DNA or RNA. When the nucleic acid molecule is in a DNA form, it may be genomic DNA or cDNA. RNA forms of the nucleic acid molecules of the present invention are generally mRNA.

Although the nucleic acid molecules of the present invention are generally in isolated form, they may be integrated into or ligated to or otherwise fused or associated with other genetic molecules such as vector molecules and in particular expression vector molecules. Vectors and expression vectors are generally capable of replication and, if applicable, expression in one or both of a prokaryotic cell or a eukaryotic cell. Preferably, prokaryotic cells include E. coli, Bacillus sp and Pseudomonas sp. Preferred eukaryotic cells include yeast, fungal, mammalian and insect cells.

Accordingly, another aspect of the present invention contemplates a genetic construct comprising a vector portion and a mammalian and more particularly a human NR6 gene portion, which NR6 gene portion is capable of encoding an NR6 polypeptide or a functional or immunologically interactive derivative thereof.

Preferably, the NR6 gene portion of the genetic construct is operably linked to a promoter on the vector such that said promoter is capable of directing expression of said NR6 gene portion in an appropriate cell.

In addition, the NR6 gene portion of the genetic construct may comprise all or part of the gene fused to another genetic sequence such as a nucleotide sequence encoding maltose binding protein or glutathione-5-transferase or part thereof.

The present invention extends to such genetic constructs and to prokaryotic or eukaryotic cells comprising same.

The present invention also extends to any or all derivatives of NR6 including mutants, part, fragments, portions, homologues and analogues or their encoding genetic sequence including single or multiple nucleotide or amino acid substitutions, additions and/or deletions to the naturally occurring nucleotide or amino acid sequence.

NR6 may be important for the proliferation, differentiation and survival of a diverse array of cell types. Accordingly, it is proposed that NR6 or its functional derivatives be used to regulate development, maintenance or regeneration in an array of different cells and tissues in vitro and in vivo. For example, NR6 is contemplated to be useful in modulating neuronal proliferation, differentation and survival.

Soluble NR6 polypeptides are also contemplated to be useful in the treatment of a range of diseases, injuries or abnormalities.

Membrane bound or soluble NR6 may be used in vitro on nerve cells or tissues to modulate proliferation, differentiation or survival, for example, in grafting procedures or transplantation.

As stated above, the NR6 of the present invention or its functional derivatives may be provided in a pharmaceutical composition comprising the NR6 together with one or more pharmaceutically acceptable carriers and/or diluents. In addition, the present invention contemplates a method of treatment comprising the administration of an effective amount of a NR6 of the present invention. The present invention also extends to antagonists and agonists of NR6s and their use in therapeutic compositions and methodologies.

A further aspect of the present invention contemplates the use of NR6 or its functional derivatives in the manufacture of a medicament for the treatment of NR6 mediated conditions defective or deficient.

Still a further aspect of the present invention contemplates a ligand for NR6 preferably, in isolated or recombinant form or a derivative of said ligand.

The present invention further contemplates knockout animals such as mice or other murine species for the NR6 gene including homozygous and heterozygous knockout animals. Such animals provide a particularly useful live in vivo model for studying the effects of NR6 as well as screening for agents capable of acting as agonists or antagonists of NR6.

According to this embodiment there is provided a transgenic animal comprising a mutation in at least one allele of the gene encoding NR6. Additionally, the present invention provides a transgenic animal comprising a mutation in two alleles of the gene encoding NR6. Preferably, the transgenic animal is a murine animal such as a mouse or rat.

The present invention is further described by the following non-limiting Figures and Examples.

### BRIEF DESCRIPTION OF THE DRAWINGS

FIGS. 1A-1B are diagrammatic representations showing expansion of sequenced region of the mouse NR6 gene indicating splicing patterns seen in the three forms of NR6 cDNA, NR6.1, NR6.2 and NR6.3.

FIGS. 2(1)-2(9) are representations of the nucleotide sequence of the mouse NR6 gene, containing exons encoding the cDNA from nucleotide 148 encoding D50 of the cDNAs shown in SEQ ID NOs:12 and 14 to the end of the 3′ untranslated region shared by both NR6.1, NR6.2 and NR6.3. In this figure, this region encompasses nucleotides g1182 to g6617. This sequence is also defined in SEQ ID NO:28.

FIGS. 3(1)-3(25) are representations of the nucleotide sequence of the mouse genomic NR6 gene with additional 5′ sequences. The coding exons of NR6 span approximately 11 kb of the mouse genome. There are 9 coding exons separated by 8 introns:

| exon 1 | at least 239 | nt | intron 1 | 5195 nt |
|---|---|---|---|---|
| exon 2 | 282 | nt | intron 2 | 214 nt |
| exon 3 | 130 | nt | intron 3 | 107 nt |
| exon 4 | 170 | nt | intron 4 | 1372 nt |
| exon 5 | 158 | nt | intron 5 | 68 nt |
| exon 6 | 169 | nt | intron 6 | 2020 nt |
| exon 6 | 188 | nt | intron 7 | 104 nt |
| exon 8 | 43 | nt | intron 8 | 181 nt |
| exon 9 | 252 | nt | | |

Exon 1 encoding the signal sequence, exon 2 the Ig-like domain, exons 3 to 6 the haemopoietin domain. Exons 7, 8 and 9 are alternatively spliced.

FIG. 4 is a diagrammatic representation showing the genomic structure of murine NR6.

FIG. 5 is a diagrammatic representation showing targetting of the NR6 locus by homologous recombination.

FIGS. 6(1)-6(6) are representations of a comparison of human and mouse NR6 cDNA sequences.

FIGS. 7(1)-7(6) are representations of a comparison of human and mouse NR6 protein sequences.

FIGS. 8(a)-8(c) are representations showing transient expression of C-terminal FLAG tagged human NR6 in 293T cells. (A) Biosensor response, M2 immobilised; (B) SDS PAGE/silver staining analysis of M2 eluted fractions; and (C) Western blot analysis of M2 eluted fractions.

FIG. 9 a photographic representation showing biosensor analysis of supernatant fluid from each of clones CHO C′ FLAG human NR6 clone #30, CHO N′ FLAG human NR6 clone #23 and 293T C′ FLAG human NR6 clone #38 (lanes 1-3, respectively).

Single and three letter abbreviations for amino acid residues used in the specification are summarised in Table 2:

TABLE 2

| | Three-letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any residue | Xaa | X |

TABLE 3
SUMMARY OF SEQ ID NO.

| | SEQ ID |
|---|---|
| Sequence | NO. |
| Amino acid sequence WSXWS | 1 |
| Oligonucleotide primers and probes listed in Example 1 | 2-11 |
| Nucleotide sequence of NR6.1¹ | 12 |
| Amino acid sequence of NR6.1 | 13 |
| Nucleotide sequence of NR6.2² | 14 |
| Amino acid sequence of NR6.2 | 15 |
| Nucleotide sequence of NR6.3³ | 16 |
| Amino acid sequence of NR6.3 | 17 |
| Nucleotide sequence of products generated by 5′ RACE of brain | 18 |
| cDNA using NR6 specific primers⁴ | |
| Amino acid sequence of SEQ ID NO: 18 | 19 |
| Nucleotide sequence unique to 5′ RACE of brain cDNA | 20 |
| Amino acid sequence for SEQ ID NO: 20 | 21 |
| Unspliced murine NR6 nucleotide sequence | 22 |
| PCR product for human NR6 | 23 |
| Nucleotide sequence of clone HFK-66 encoding human NR6 | 24 |
| Amino acid sequence of SEQ ID NO: 24 | 25 |
| Oligonucleotide sequences UP1 and LP1, respectively | 26-27 |
| Genomic nucleotide sequence of murine NR6 | 28 |
| Amino acid sequence of SEQ ID NO: 28 | 29 |
| Murine NR6.1 oligonucleotide primers | 30, 31 |
| Murine IL-3 signal sequence | 32 |
| Linker sequence for mouse IL-3 signal sequence and | 33-35 |
| FLAG epitope | |
| Genomic nucleotide sequence of murine NR6 containing | 38 |
| additonal 5′ sequence | |
| Oligonucleotide 2199 and 2200, respectively | 36, 37 |
| N-terminal region of NR6 | 39 |
| Oligonucleotide sequences | 40-42 |
| Nucleotide sequence of NR6 | 43 |
| Amino acid sequence of NR6 | 44 |
| Oligonucleotide sequences | 45-54 |

The polyadenylation signal AATAAATAAA (SEQ ID NO: 58) is at nucleotide position 1574 to 1583 of SEQ ID NO: 12; the coding sequences of NR6.1 (SEQ ID NO: 12) and NR6.2 (SEQ ID NO: 14) are identical to the codon encoding Q407, which represents the end of an exon. NR6.1 splices out an exon present only in NR6.2 and uses a different reading frame for the final exon which is shared with NR6.2; this corresponds to amino acids VLPAKL at amino acid residue positions 408-413 of SEQ ID NO: 13. The region of 3′-untranslated DNA shared by NR6.1, NR6.2 and NR6.3 is from nucleotide 1363 to 1598 of SEQ ID NO: 12. The WSXWS motif is at amino acid residues 330 to 334 of SEQ ID NO: 13.

The polyadenylation signal AATAAATAAA (SEQ ID NO: 58) is at nucleotide positions 1618 to 1627 of SEQ ID NO: 14. The WSXWS motif is at amino acid residues 330 to 334 of SEQ ID NO: 15. NR6.1 and NR6.2 are identical to the codon encoding Q407 which represents the end of an exon. NR6.2 splices in an exon beginning at nucleotide 1348 and ends at nucleotide 1388 of SEQ ID NO: 14 (corresponding to the coding sequence for amino acid residue D408 to residue G422 of SEQ ID NO: 15). The region of 3′ untranslated DNA shared by NR6.1, NR6.2 and NR6.3 is from nucleotide position 1407 to 1641 of SEQ ID NO: 14.

The WSXWS motif is at amino acid residues 24-28 of SEQ ID NO: 17. The polyadenylation signal AATAAATAAA (SEQ ID NO: 58) is from nucleotide 863 to 872 of SEQ ID NO: 16. Amino acids 1-101 of NR6.3 (SEQ ID NO: 17) are identical to amino acids 307-407 of NR 6.1 (SEQ ID NO: 13) and NR6.2 (SEQ ID NO: 15). The codon encoding Q407 of SEQ ID NO: 13 or Q101 of SEQ ID NO: 17 represents the end of an exon. NR6.3 fails to splice from this position and, therefore, translation continues through the intron, giving rise to the C-terminal protein region from amino acid residues 102-155 of SEQ ID NO: 17. The region of 3′ untranslated DNA shared by NR6.1, NR6.2 and NR6.3 is from nucleotide 650-887 of SEQ ID NO 16.

The nucleotide sequence is identical to NR6.1, NR6.2 and NR6.3 from nucleotide C151, the first nucleotide for Pro51. The numbering from this nucleotide is the same as for SEQ ID NO:14 and 16. The 5′ of this point is unique to the products generated by 5′ RACE not being found in NR6.1, NR6.2 and NR6.3 and is represented in SEQ ID NOs:20 and 21.

Structure of the murine genomic NR6 locus. The coding exons of NR6 span approximately 11 kb of the mouse genome. There are 9 coding exons separated by 8 introns:

| exon 1 | at least 239 | nt | intron 1 | 5195 nt |
|---|---|---|---|---|
| exon 2 | 282 | nt | intron 2 | 214 nt |
| exon 3 | 130 | nt | intron 3 | 107 nt |
| exon 4 | 170 | nt | intron 4 | 1372 nt |
| exon 5 | 158 | nt | intron 5 | 68 nt |
| exon 6 | 169 | nt | intron 6 | 2020 nt |
| exon 7 | 188 | nt | intron 7 | 104 nt |
| exon 8 | 43 | nt | intron 8 | 181 nt |
| exon 9 | 252 | nt | | |

Exon 1 encodes the signal sequence, exon 2 the Ig-like domain, exons 3 to 6 the hemopoietin domain. Exons 7, 8 and 9 are alternatively spliced.

The NRG molecules of the present invention have a range of utilities referred to in the subject specification. Additional utilities include:

1. Identification of molecules that interact with NR6. These may include:

a) a corresponding ligand using standard orphan receptor techniques (26),

b) monoclonal antibodies that act either as receptors antagonists or agonists,

c) mimetic or antagonistic peptides isolated using phage display technology (27,28),

d) small molecule natural products that act either as antagonists or agonists.

2. Development of diagnostics to detect deletions/rearrangements in the NR6 gene.

The NR6 knock-out mice studies described herein provide a useful model for this utility. There are also applications in the field of reproduction. For example, people can be tested for their NR6 status. NR6+/−carriers might be expected to give rise to offspring with developmental problems.

### EXAMPLE 1

### Oligonucleotides

| | |
|---|---|
| M116: | |
| 5′ ACTCGCTCCAGATTCCCGCCTTTT 3′ | [SEQ ID NO: 2] |
| M108: | |
| 5′ TCCCGCCTTTTTCGACCCATAGAT 3′ | [SEQ ID NO: 3] |
| M159: | |
| 5′ GGTACTTGGCTTGGAAGAGGAAAT 3′ | [SEQ ID NO: 4] |
| M242: | |
| 5′ CGGCTCACGTGCACGTCGGGTGGG 3′ | [SEQ ID NO: 5] |
| M112: | |
| 5′ AGCTGCTGTTAAAGGGCTTCTC 3′ | [SEQ ID NO: 6] |
| WSDWS | |
| 5′ (A/G)CTCCA(A/G)TC(A/G)CTCCA 3′ | [SEQ ID NO: 7] |
| WSEWS | |
| 5′ (A/G)CTCCA(C/T)TC(A/G)CTCCA 3′ | [SEQ ID NO: 8] |
| 1944 | |
| 5′ AAGTGTGACCATCATGTGGAC 3′ | [SEQ ID NO: 9] |
| 2106 | |
| 5′ GGAGGTGTTAAGGAGGCG 3′ | [SEQ ID NO: 10] |
| 2120 | |
| 5′ ATGCCCGCGGGTCGCCCG 3′ | [SEQ ID NO: 11] |

### EXAMPLE 2

### Isolation of Initial NR6 cDNA Clones Using Oligonucleotides Designed Against the Conserved WSXWS Motif Found in Members of the Haemopoietin Receptor Family

(i) A commercial adult mouse testis cDNA library cloned into the UNI-ZAP bacteriophage (Stratagene, Calif., USA; Catalogue numbers 937 308) was used to infect Escherichia coli of the strain LE392. Infected bacteria were grown on twenty 150 mm agar plates, to give approximately 50,000 plaques per plate. Plaques were then transferred to duplicate 150 mm diameter nylon membranes (Colony/Plaque Screen, NEN Research Products, MA, USA), bacteria were lysed and the DNA was denatured and fixed by autoclaving at 100° C. for 1 min with dry exhaust. The filters were rinsed twice in 0.1% (w/v) sodium dodecyl sulfate (SDS), 0.1×SSC(SSC is 150 mM sodium chloride, 15 mM sodium citrate dihydrate) at room temperature and pre-hybridized overnight at 42° C. in 6×SSC containing 2 mg/ml bovine serum albumin, 2 mg/ml Ficoll, 2 mg/ml polyvinylpyrrolidone, 100 mM ATP, 10 mg/ml tRNA, 2 mM sodium pyrophosphate, 2 mg/ml salmon sperm DNA, 0.1% (w/v) SDS and 200 mg/ml sodium azide. The pre-hybridisation buffer was removed. 1.2 μg of the degenerate oligonucleotides for hybridization (WSDWS; Example 1) were phosphorylated with T4 polynucleotide kinase using 960 mCi of y³²P-ATP (Bresatec, S. A., Australia). Unincorporated ATP was separated from the labelled oligonucleotide using a pre-packed gel filtration column (NAP-5; Pharmacia, Uppsala, Sweden). Filters were hybridized overnight at 42° C. in 80 ml of the prehybridisation buffer containing 0.1% (w/v) SDS, rather than NP40, and 10⁶-10⁷ cpm/ml of labelled oligonucleotide. Filters were briefly rinsed twice at room temperature in 6×SSC, 0.1% (v/v) SDS, twice for 30 min at 45° C. in a shaking waterbath containing 1.5 l of the same buffer and then briefly in 6×SSC at room temperature. Filters were then blotted dry and exposed to autoradiographic film at −70° C. using intensifying screens, for 7-14 days prior to development.

Plaques that appeared positive on orientated duplicate filters were picked, eluted in 1 ml of 100 mM NaCl, 10 mM MgCl₂, 10 mM Tris.HCl pH7.4 containing 0.5% (w/v) gelatin and 0.5% (v/v) chloroform and stored at 4° C. After 2 days LE392 cells were infected with the eluate from the primary plugs and replated for the secondary screen. This process was repeated until hybridizing plaques were pure.

Once purified, positive cDNAs were excised from the ZAP II bacteriophage according to the manufacturer's instructions (Stratagene, Calif., USA) and cloned into the plasmid pBluescript. A CsCl purified preparation of the DNA was made and this was sequenced on both strands. Sequencing was performed using an Applied Biosystems automated DNA sequencer, with fluorescent dideoxynucleotide analogues according to the manufacturer's instructions. The DNA sequence was analysed using software supplied by Applied Biosystems.

Two clones isolated from the mouse testis cDNA library shared large regions of nucleotide sequence identity 68-1 and 68-2 and appeared to encode a novel member of the haemopoietin receptor family and the inventors gave the putative receptor the working name “NR6”.

(ii) In a parallel series of experiments, a commercial mouse brain cDNA library (STRATAGENE #967319, Balb/c day-20, whole brain cDNA/Uni-ZAP XR Vector) was used to infect E. coli strain XL1-Blue MRF′. Infected bacteria were grown on 90×135 mm square agar plates to give about 25,000 plaques per plate. Plaques were then transferred to positively charged nylon membranes, Hybond-N(+) (Amersham RPN 203B), bacteria were lysed and the DNA was denatured with denaturing 0.5 M NaOH, 1.5 M NaCl at room temperature for 7 min. The membranes were neutralized with 0.5 M Tris-HCL pH7.2, 1.5 M NaCl, 1 mM EDTA at room temperature for 10 min before the DNA fixation by UV crosslinking.

A mixture of WSDWS and WSEWS oligonucleotide probes (SEQ ID NOs: 7 and 8) were labelled with a [α-³²P]-ATP (TOYOBO #PNK-104 Kination kit). The membranes from the mouse brain cDNA library were then hybridized with the mixture of WSDWS and WSEWS oligonucleotide probes in the Rapid Hybridization Buffer (Amersham, RPN1636) at 42° C. for 16 hours. Filters were washed with 1×SSC/0.1% (w/v) SDS at 42° C. before autoradiography. Plaques that appeared positive on orientated duplicate filters were picked and replated on E. coli, XL1-Blue MRF′ with the process of immobilisation on nylon membranes, hybridization of membranes with oligonucleotide probes, washing and autoradiography repeated until pure plaques had been obtained.

The cDNA fragment from pure positively hybridizing plaques was isolated by excision with the helper phage strain ExAssist according to the manufacturer's instructions (Stratagene, #967319). Sequencing was performed after the amplification with Ampli-Taq DNA polymerase and Taq dideoxy terminator cycle sequencing kit (Perkin Elmer, #401150) by 25 cycles of 96° C. for 10 sec, 50° C. for 5 sec, 60° C. for 4 min followed by 60° C. for 5 min with the sequencing primers on an ABI model 377 DNA sequencer.

One clone, MBC-8, from the mouse brain library shared large regions of nucleotide sequence identity with both the 68-1 and 68-2 clones isolated from the mouse testis cDNA library.

(iii) In a third series of experiments, total RNA was prepared from the mouse osteoblastic cell line, KUSA, according to the method of Chirgwin et al. (15), and poly(A)+ RNA was further purified by oligo(dT)-cellulose chromatography (Pharmacia Biotech). Complementary DNA was synthesized by oligo(dT) priming, inserted into the UniZAP XR directional cloning vector (Stratagene), and packaged into λ phage using Gigapack Gold (Stratagene), yielding 1.25×10⁷ independent clones.

Approximately 10⁶ clones were screened essentially as described in (ii) above. Briefly, probes were labeled with ³²P using T4 polynucleotide kinase and prehybridization was performed for 4 hr in the Rapid hybridization buffer (Amersham LIFE SCIENCE) at 42° C. Filters (Hybond N+, Amersham) were then hybridized for 19 hr under the same condition with the addition of ³²P-labeled WSXWS mix oligonucleotides and washed 3 times. The final wash was for 30 min in 1×SSPE, 0.1% (w/v) SDS at 42° C. Filters were then exposed with an intensifying screen to Kodak X-OMAT AR film for 5 days.

Isolated clones were subjected to the in vivo excision of pBluescript SK(−) phagemid (Stratagene), and plasmid DNA was prepared by the standard method. DNA sequences were determined using an ABI PRISM 377 DNA Sequencer (Perkin Elmer) with appropriate synthetic oligonucleotide primers. A clone pKUSA166 shared large regions of nucleotide sequence identity with the MBC-8, 68-1 and 68-2 clones isolated from the mouse brain and testis cDNA libraries.

### EXAMPLE 3

### Isolation of Further NR6 cDNA Clones Using Probes Specific for NR6

(i) In order to identify other cDNA libraries containing cDNA clones for NR6, the inventors performed PCR upon 1 μl aliquots of λ-bacteriophage cDNA libraries made from mRNA from various human tissues and using oligonucleotides 2070 and 2057, designed from the sequence of 68-1 and 68-2, as primers. Reactions contained 5 μl of 10× concentrated PCR buffer (Boehringer Mannheim GmbH, Mannheim, Germany), 1 μl of 10 mM dATP, dCTP, dGTP and dTTP, 2.5 μl of the oligonucleotides HYB2 and either T3 or T7 at a concentration of 100 mg/ml, 0.5 μl of Taq polymerase (Boehringer Mannheim GmbH) and water to a final volume of 50 μl. PCR was carried out in a Perkin-Elmer 9600 by heating the reactions to 96° C. for 2 min and then for 25 cycles at 96° C. for 30 sec, 55° C. for 30 sec and 72° C. for 2 min. PCR products were resolved on an agarose gel, immobilized on a nylon membrane and hybridized with ³²P-labelled oligonucleotide 1943 (SEQ ID NO:42).

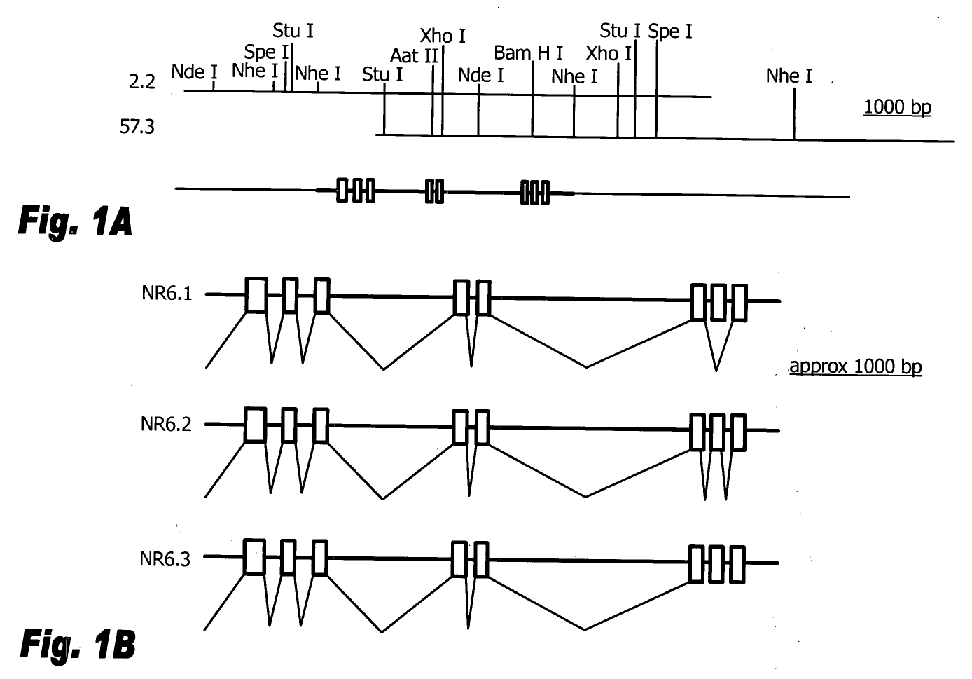

In addition to the original library, a mouse brain cDNA library appeared to contain NR6 cDNAs. These were screened using a ³²P-labelled oligonucleotides 1944, 2106, 2120 (Example 1) or with a fragment of the original NR6 cDNA clone from 68-1 (nucleotide 934 to the end of NR6.1 in FIGS. 1A-1B) labelled with ³²P using a random decanucleotide labelling kit (Bresatec). Conditions used were similar to those described in (i) above except that for the labelled oligonucleotides, filters were washed at 55° C. rather than 45° C., while for the NR6 cDNA fragment prehybridization and hybridization was carried out in 2×SSC and filters were washed at 0.2×SSC at 65° C. Again, as described in (i) above, positively hybridising plaques were purified, the cDNAs were recovered and cloned into plasmids pBluescript II or pUC19. Independent cDNA clones were sequenced on both strands.

Using this procedure, 6 further clones, 68-5, 68-35, 68-41, 68-51, 68-77 and 73-23, contained large regions of sequence identity with 68-1, 68-2, MBC-8 and pKUSA166.

In a parallel series of experiments, further screening was performed with hybridization probes prepared from the 1.7 kbp EcoRI-XhoI fragment excised from pKUSA166. This fragment was excised and labeled with ³²P by using T7QuickPrime Kit (Pharmacia Biotech). Approximately 6×10⁵ clones were screened. Hybond N+filters (Amersham) were first prehybridized for 4 hr at 42° C. in 50% (v/v) formamide, 5×SSPE, 5× Denhardt's solution, 0.1% (w/v) SDS, and 0.1 mg/ml denatured salmon sperm DNA. Hybridization was for 16 hours under the same conditions with the addition of ³²P-labelled NR6-cDNA fragment probes. Finally the filters were washed once for 1 hr in 0.2×SSC, 0.1% (w/v) SDS at 68° C. Eight clones were isolated, and phage clones were subjected to the in vivo excision of the pBluescript SK(−) phagemid (Stratagene). The plasmid DNAs were prepared by the standard method. DNA sequences were determined by an ABI PRISM 377 DNA Sequencer using appropriate synthetic oligonucleotide primers.

Using this procedure 8 further clones from the KUSA library contained large regions of sequence identity with 68-1, 68-2, MBC-8, pKUSA166, 68-5, 68-35, 68-41, 68-51, 68-77 and 73-23 were isolated.

### EXAMPLE 4

### Isolation of Genomic DNA Encoding NR6

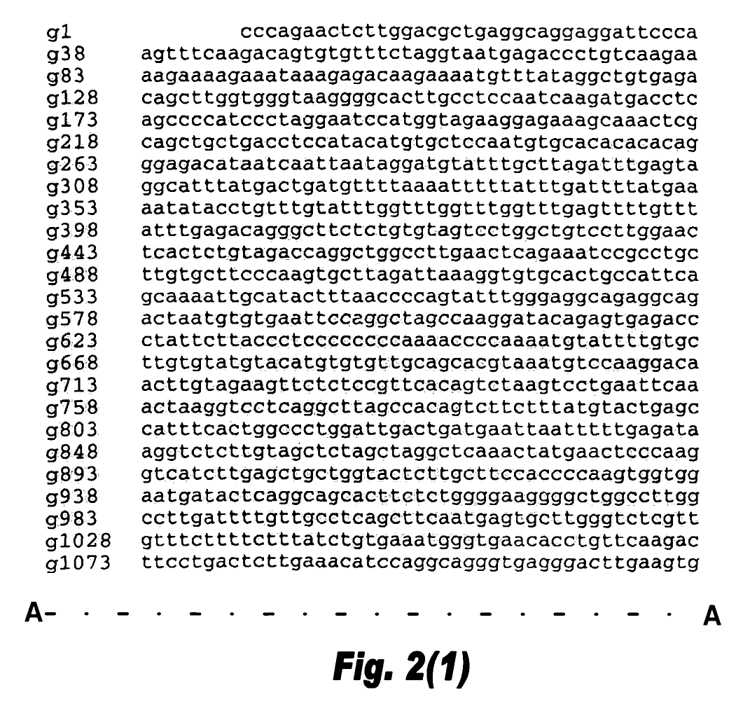

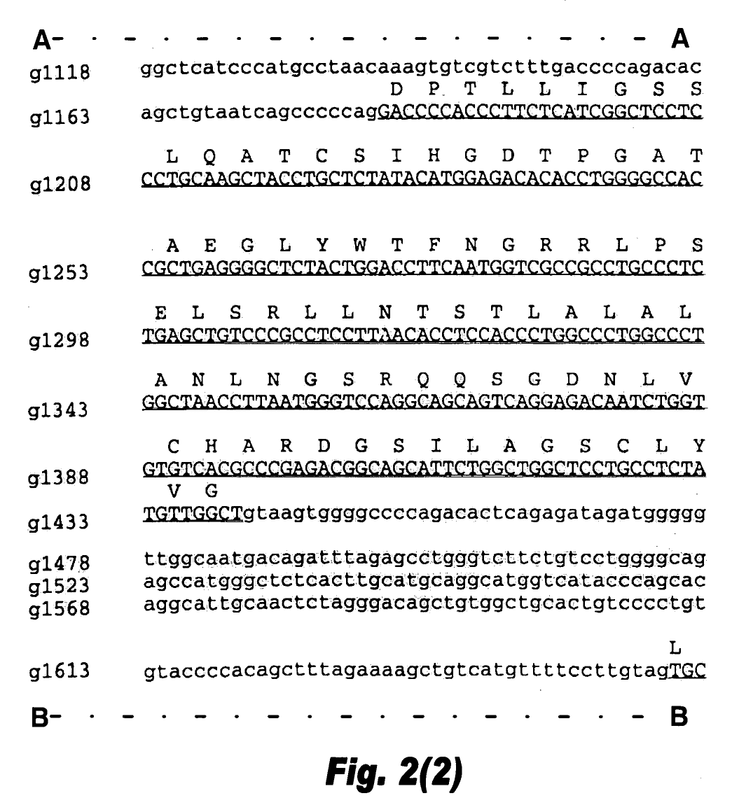

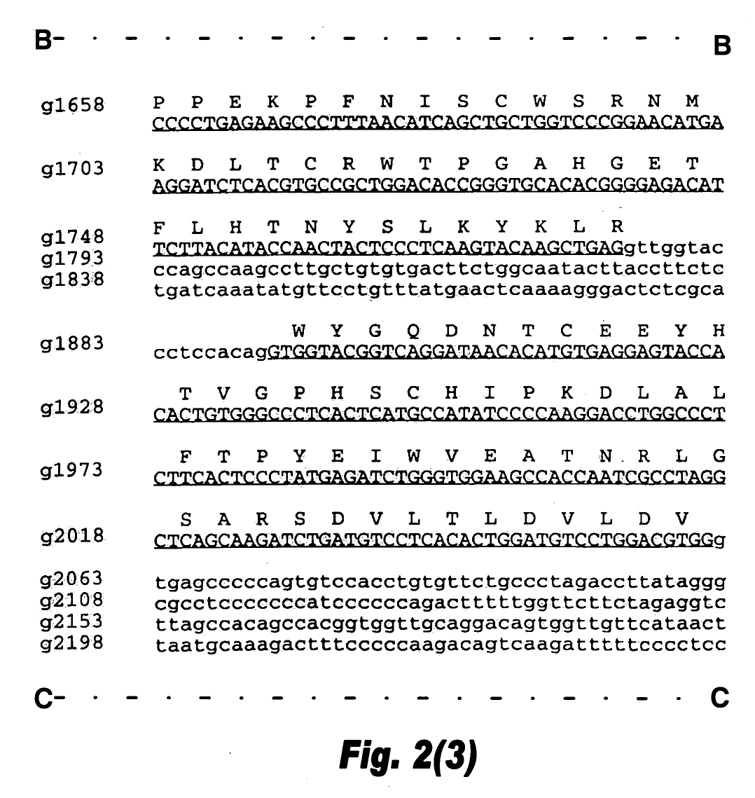

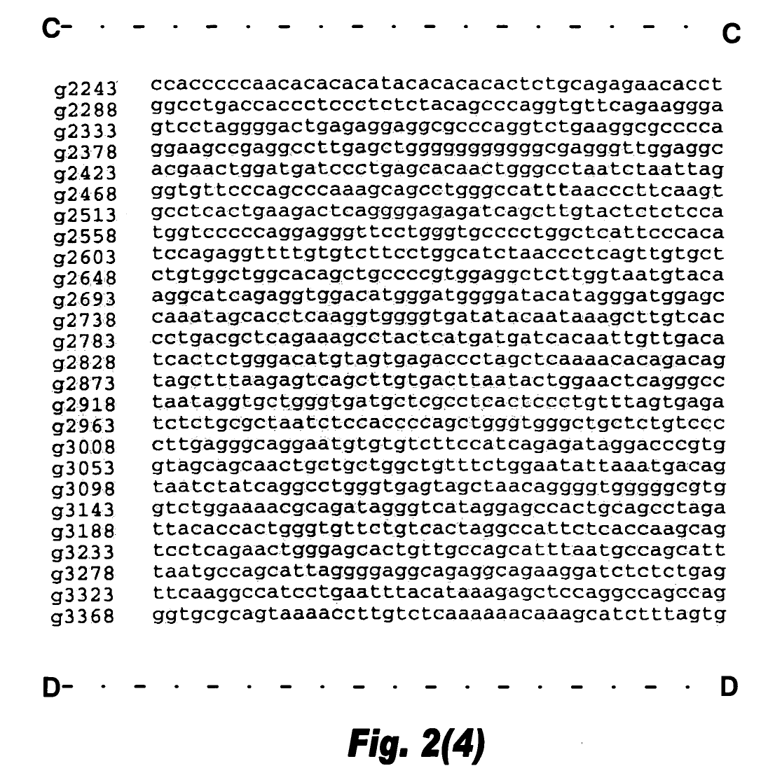

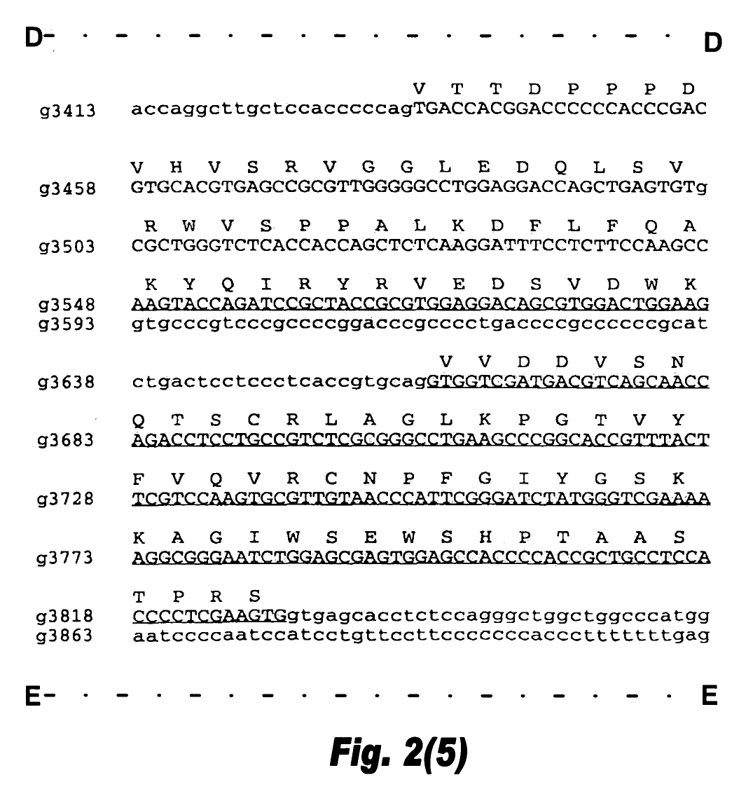

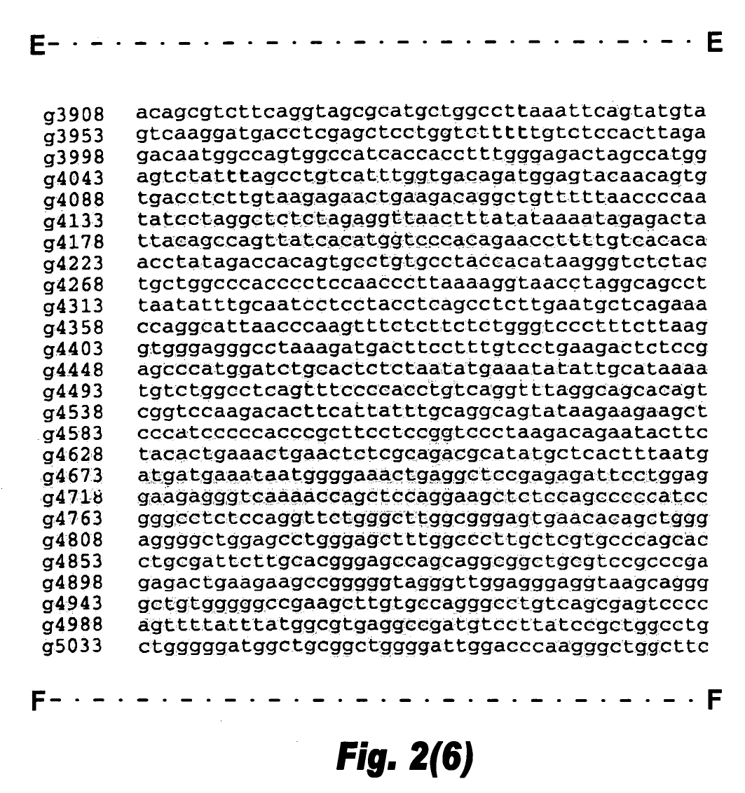

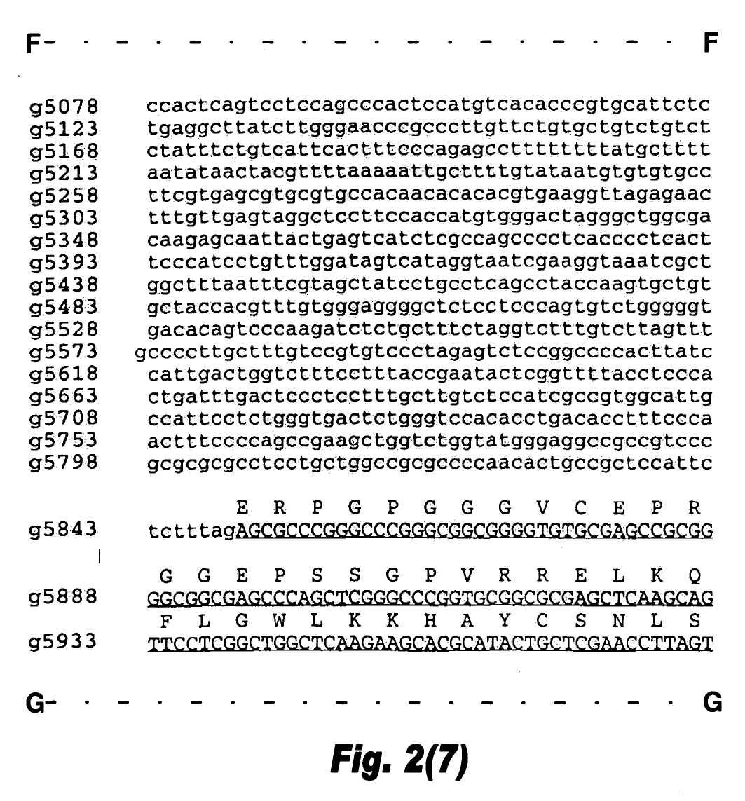

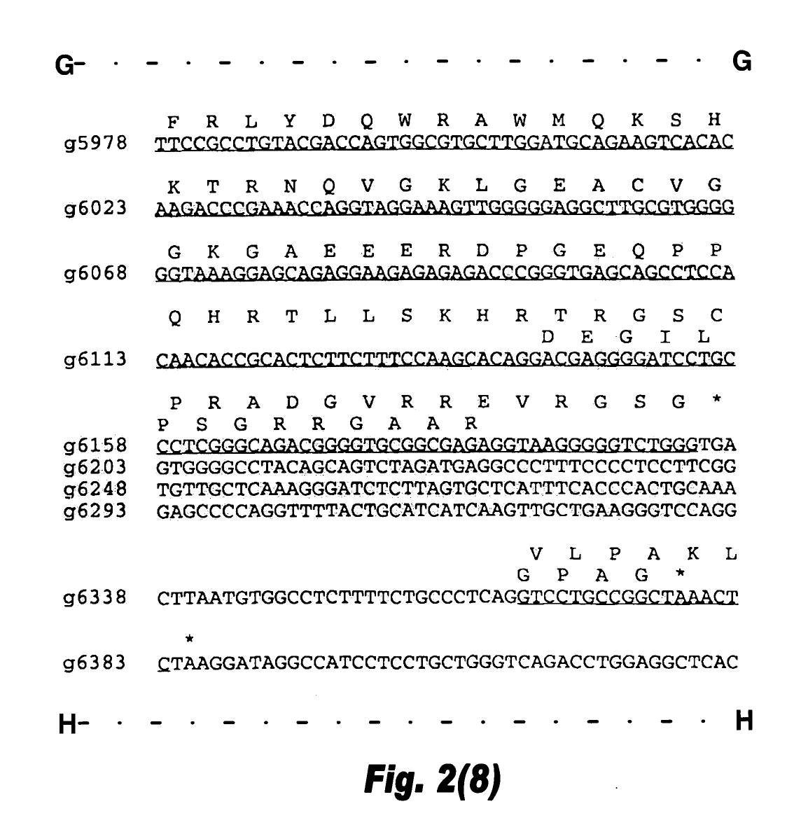

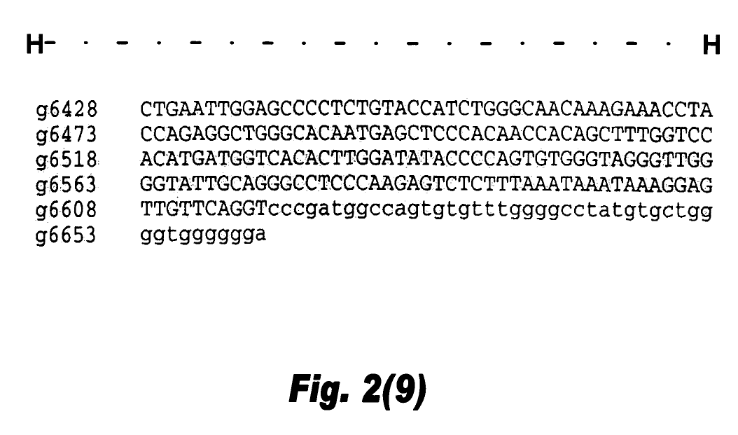

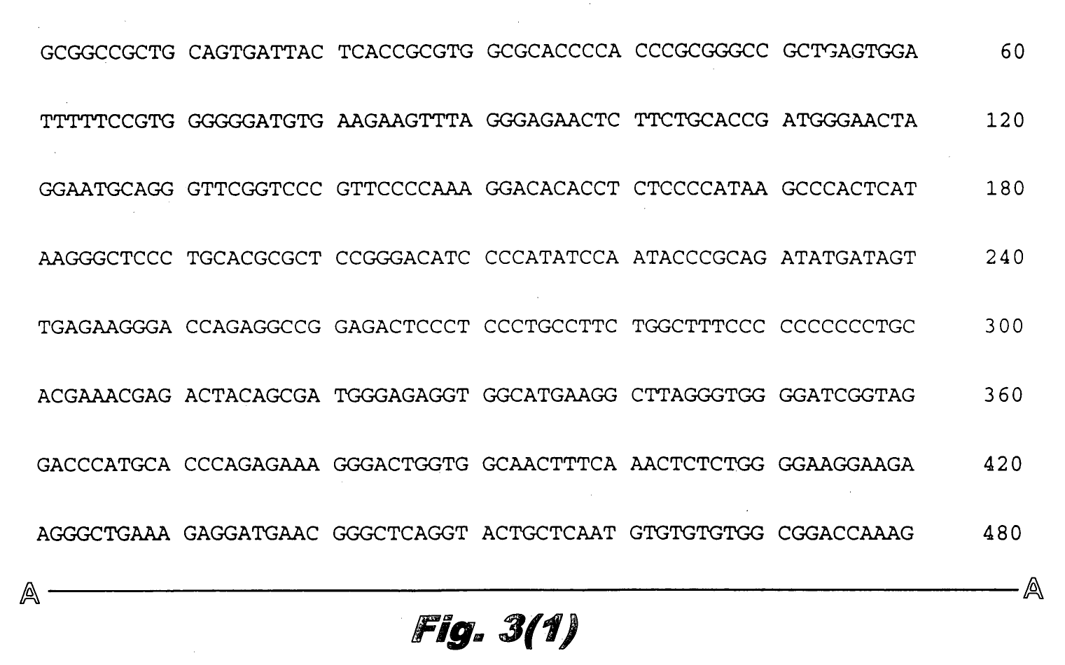

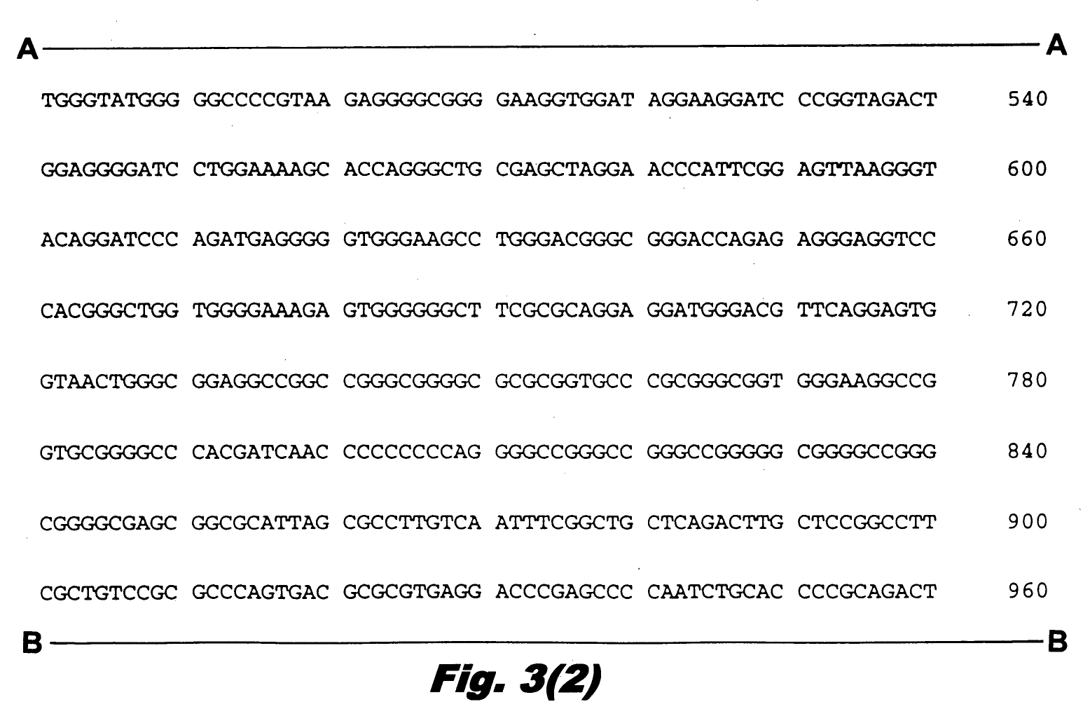

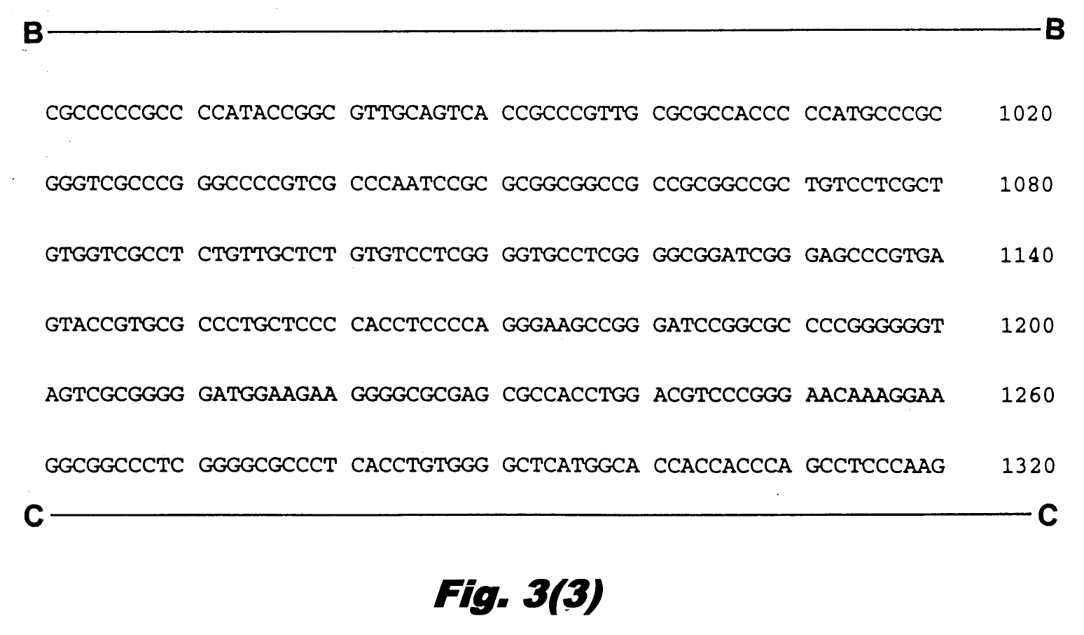

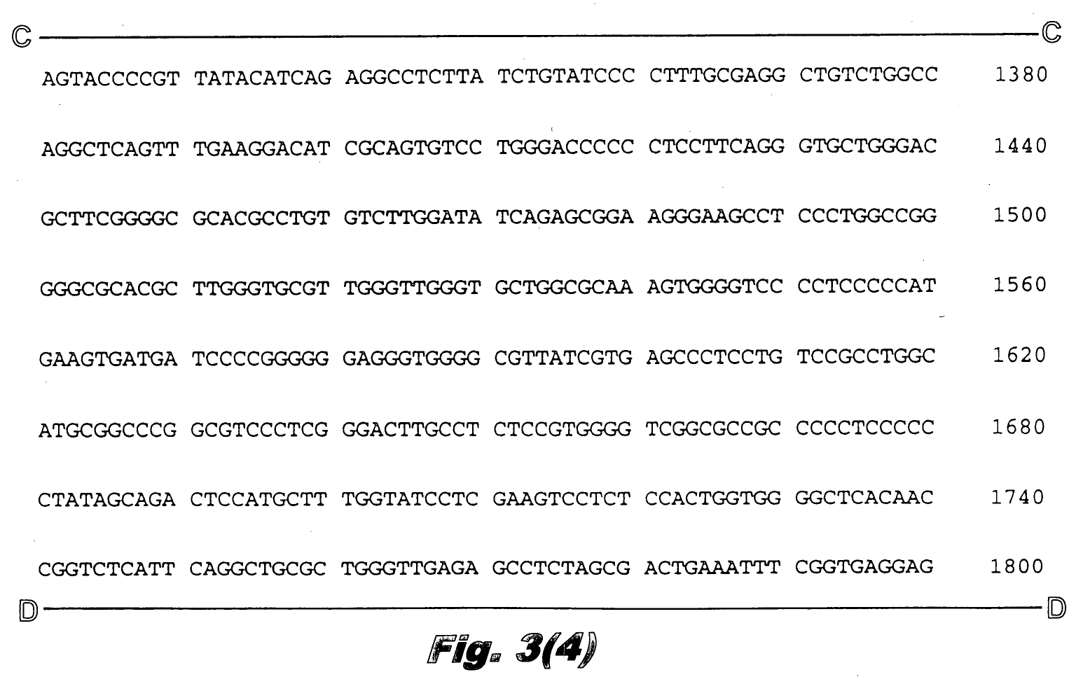

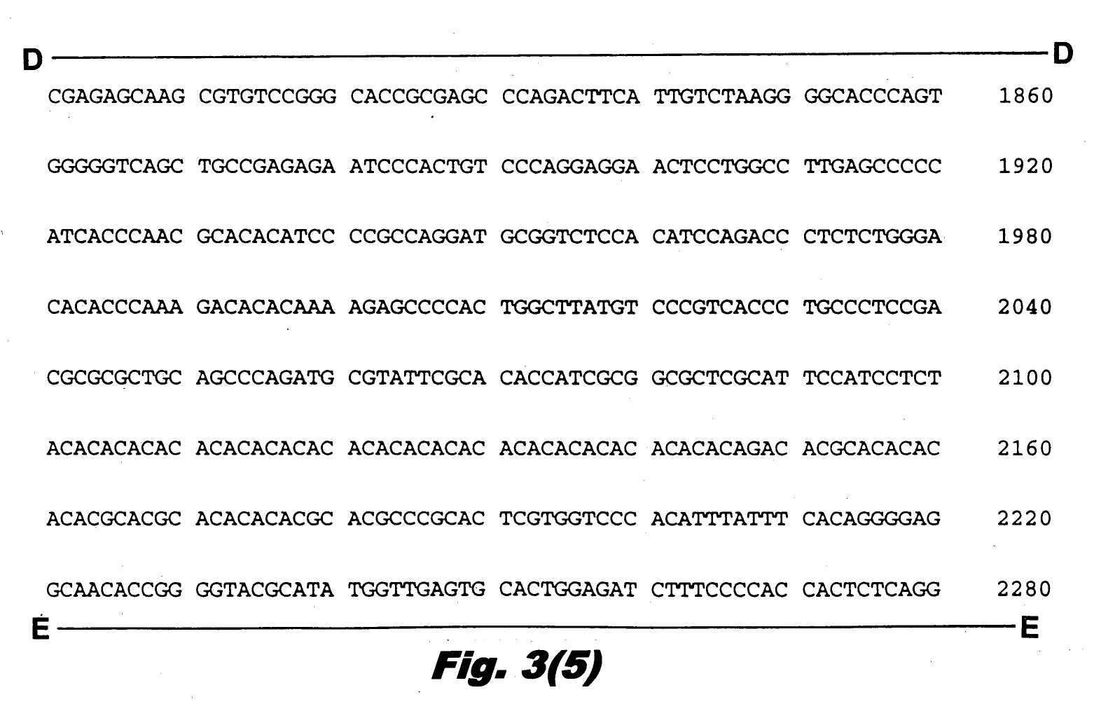

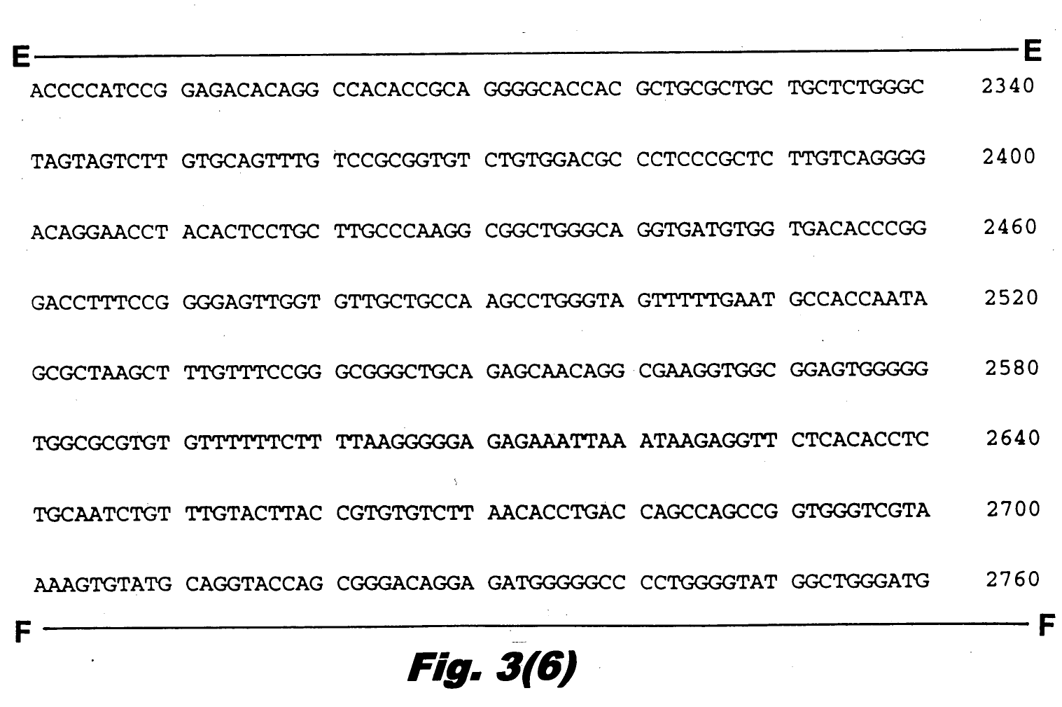

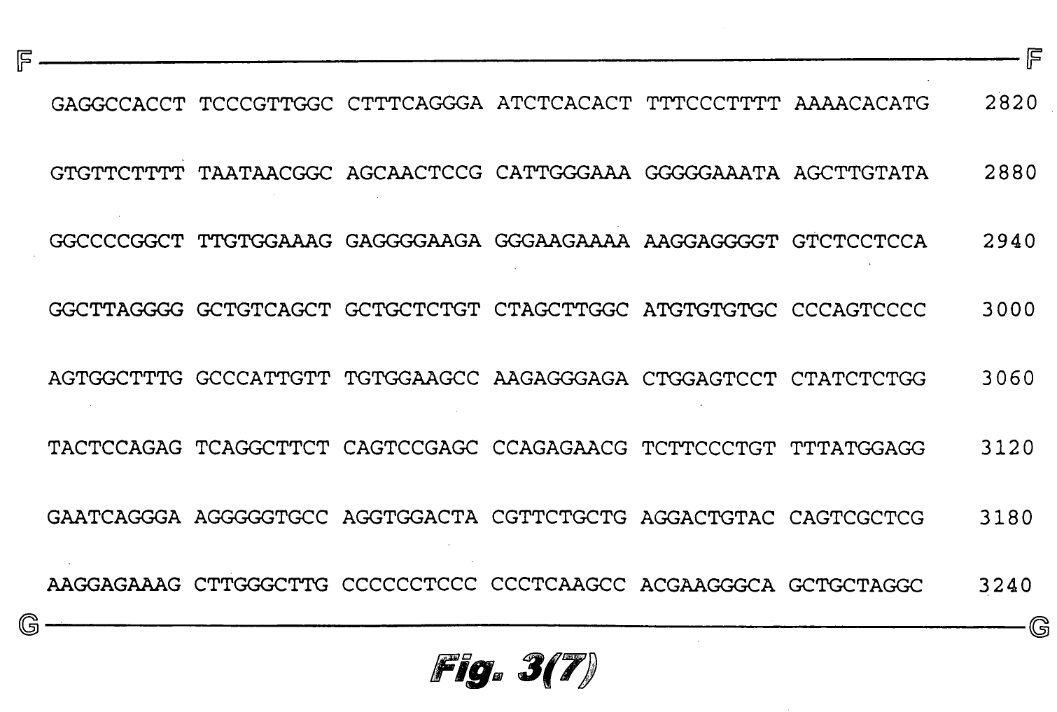

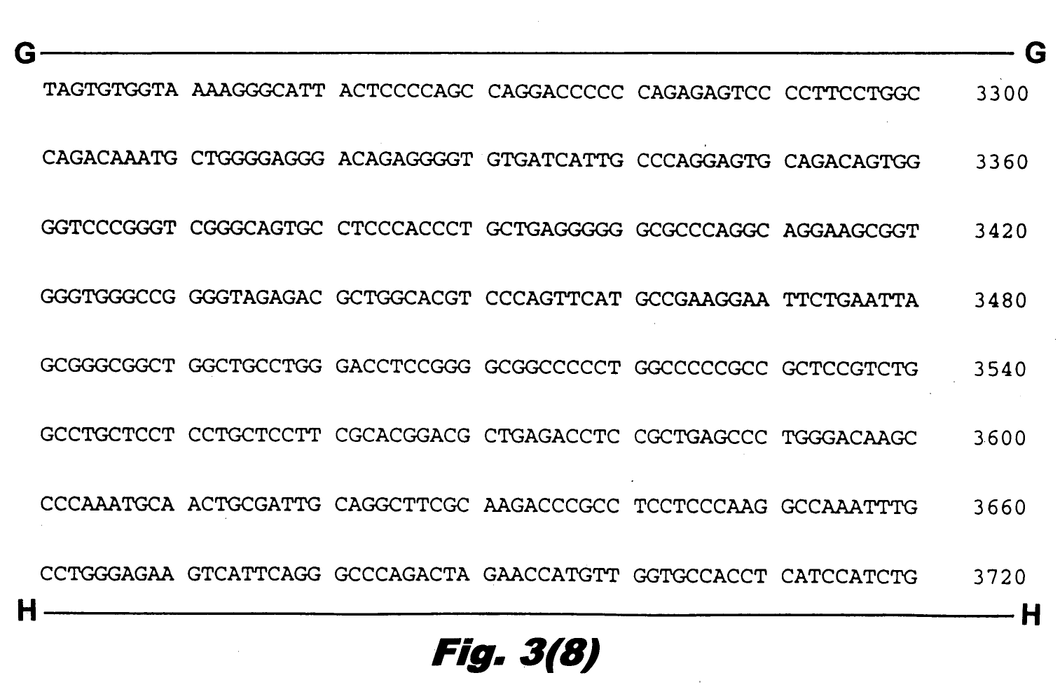

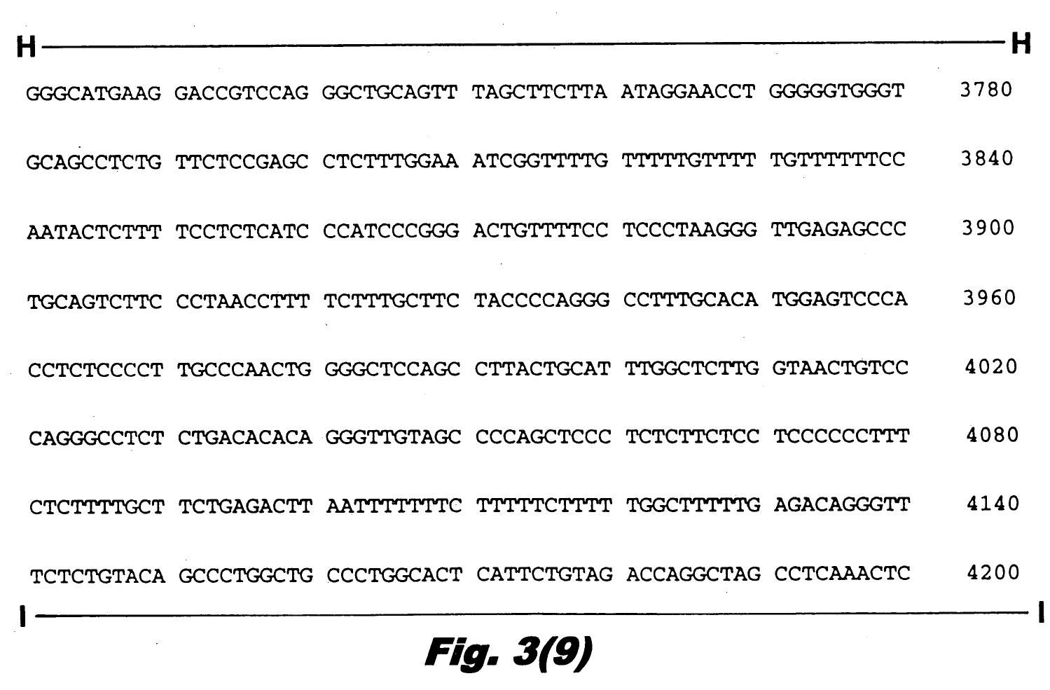

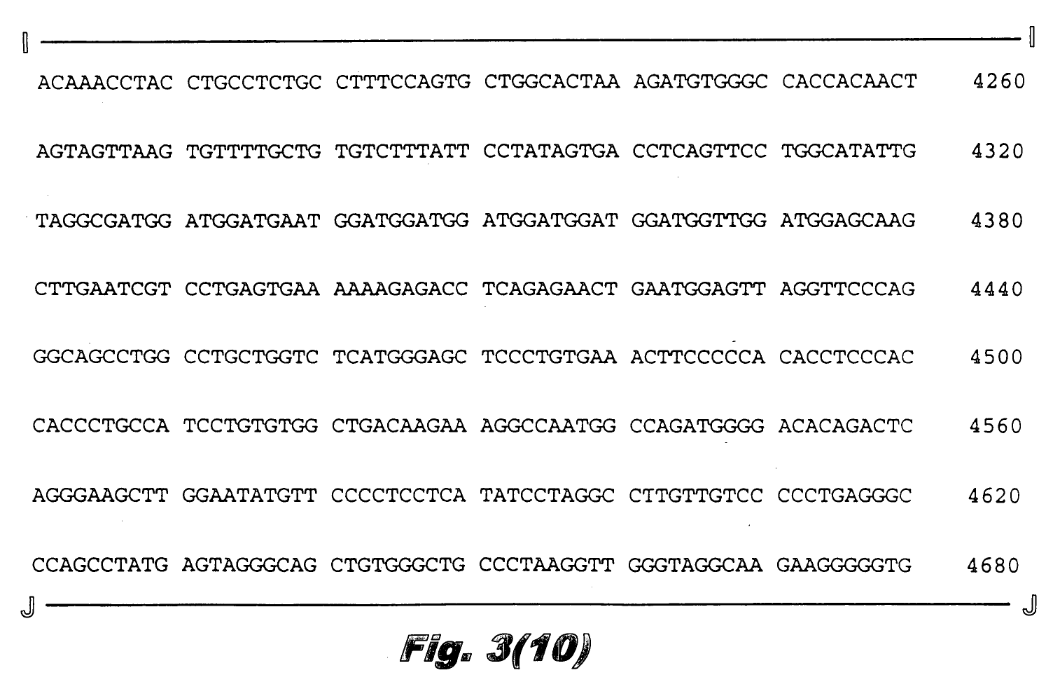

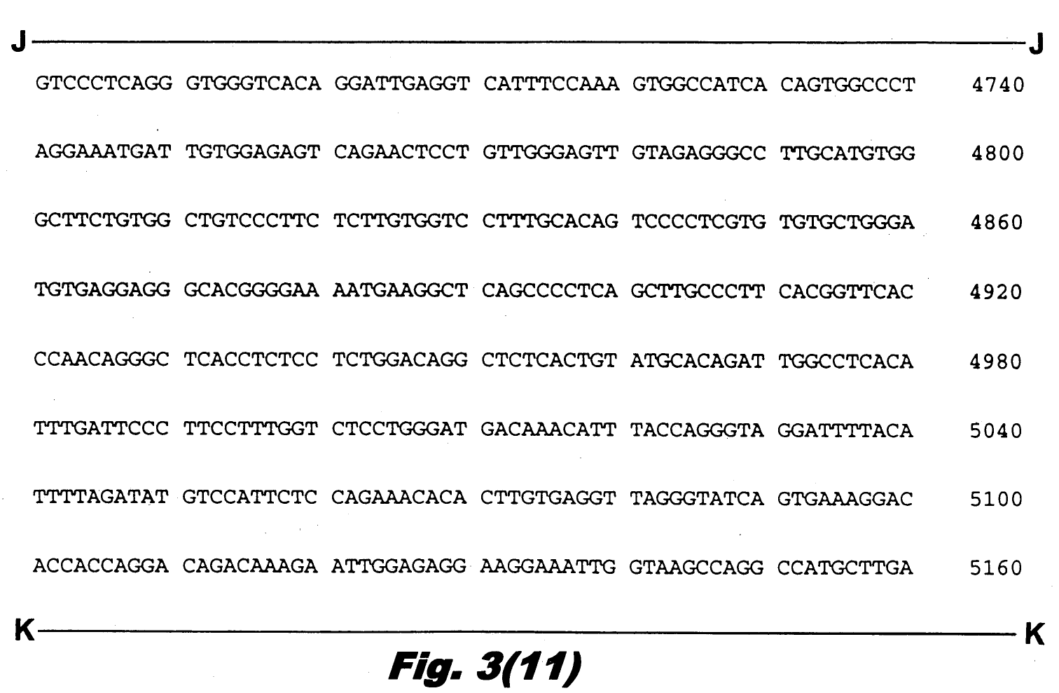

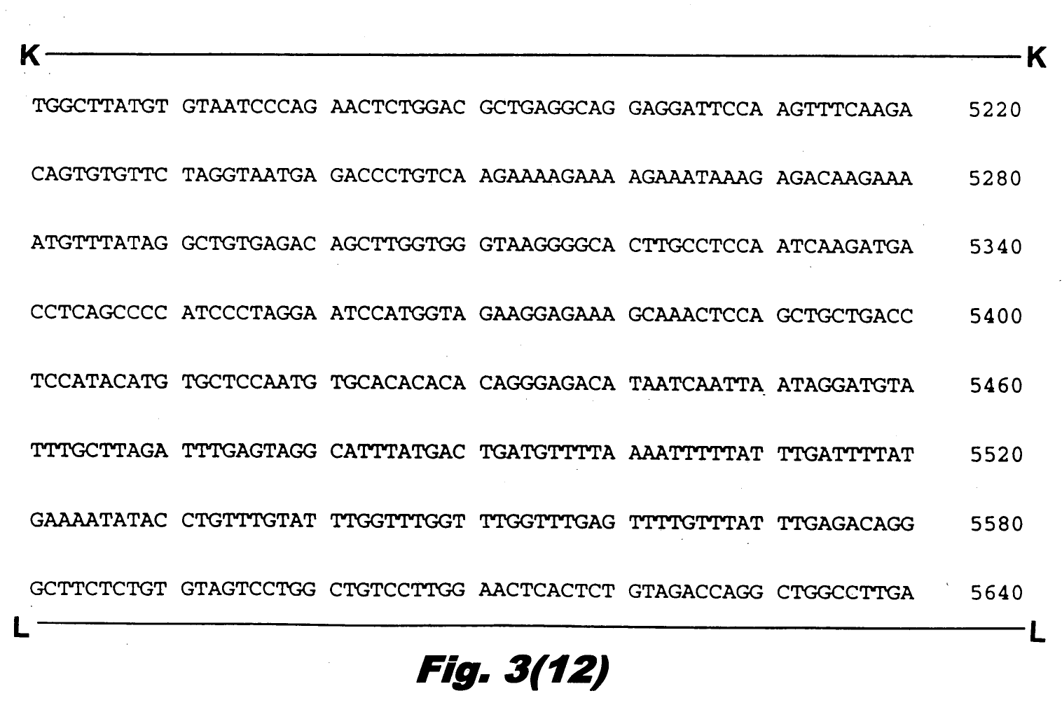

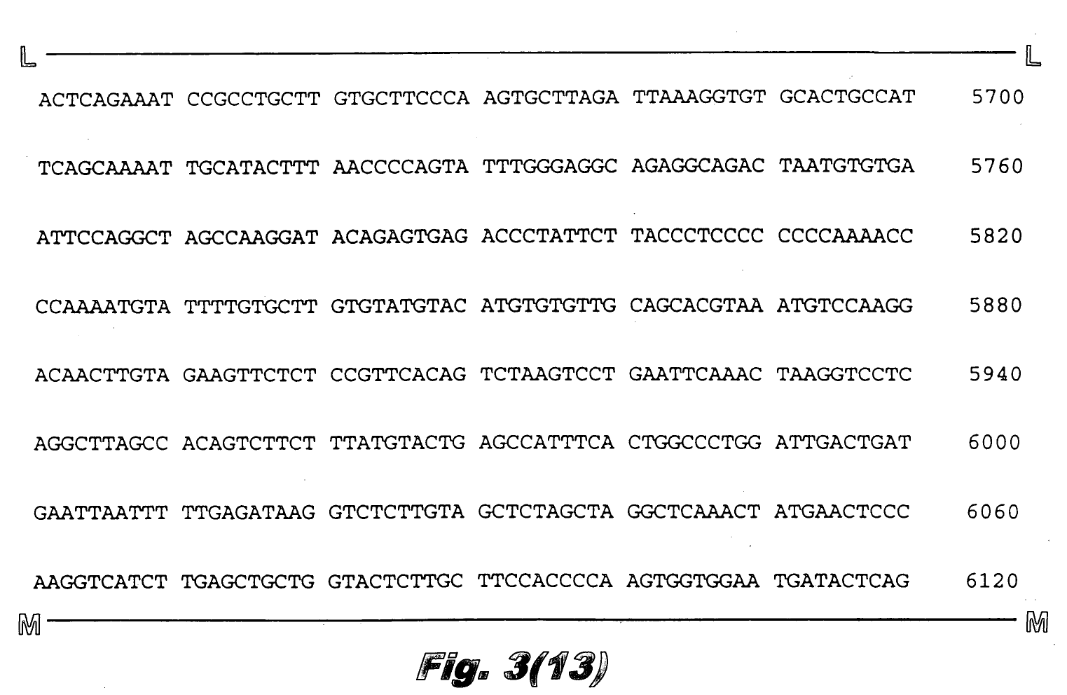

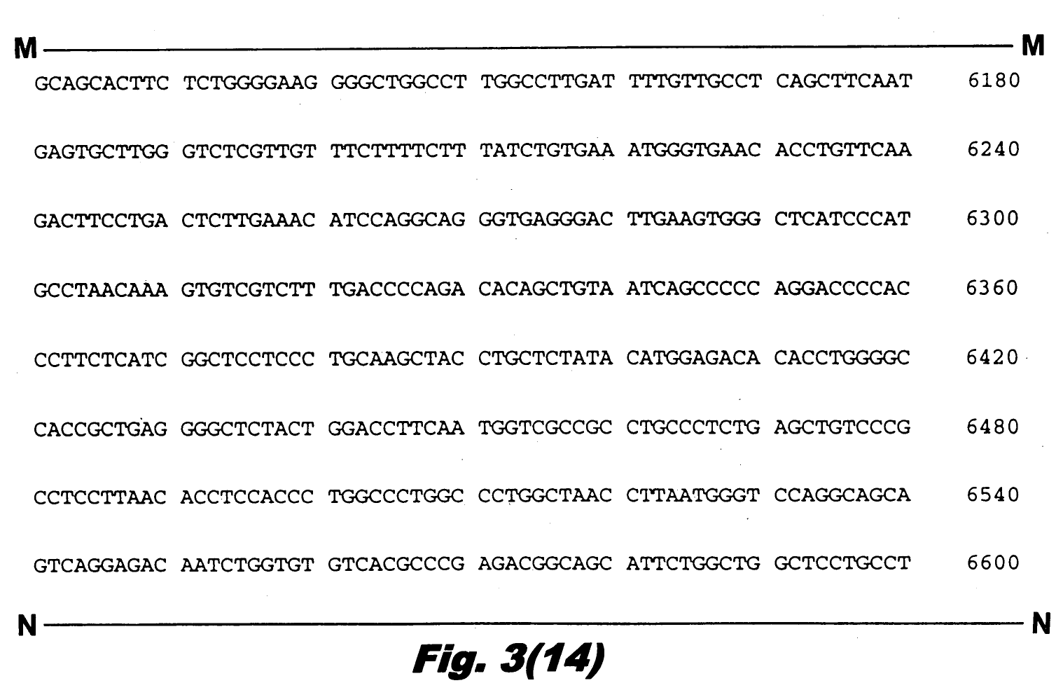

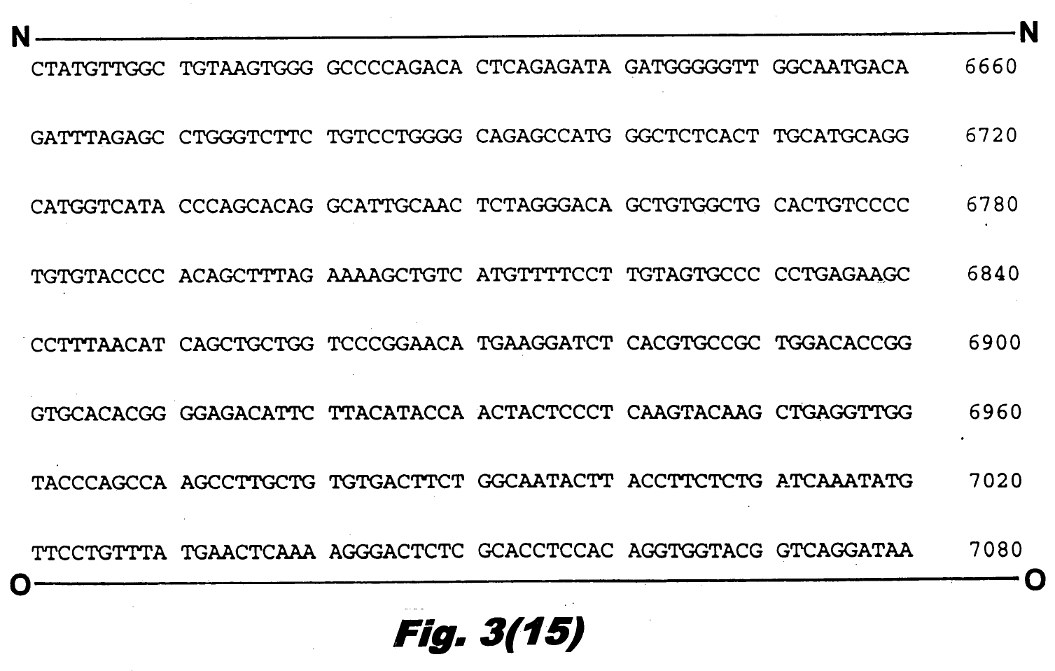

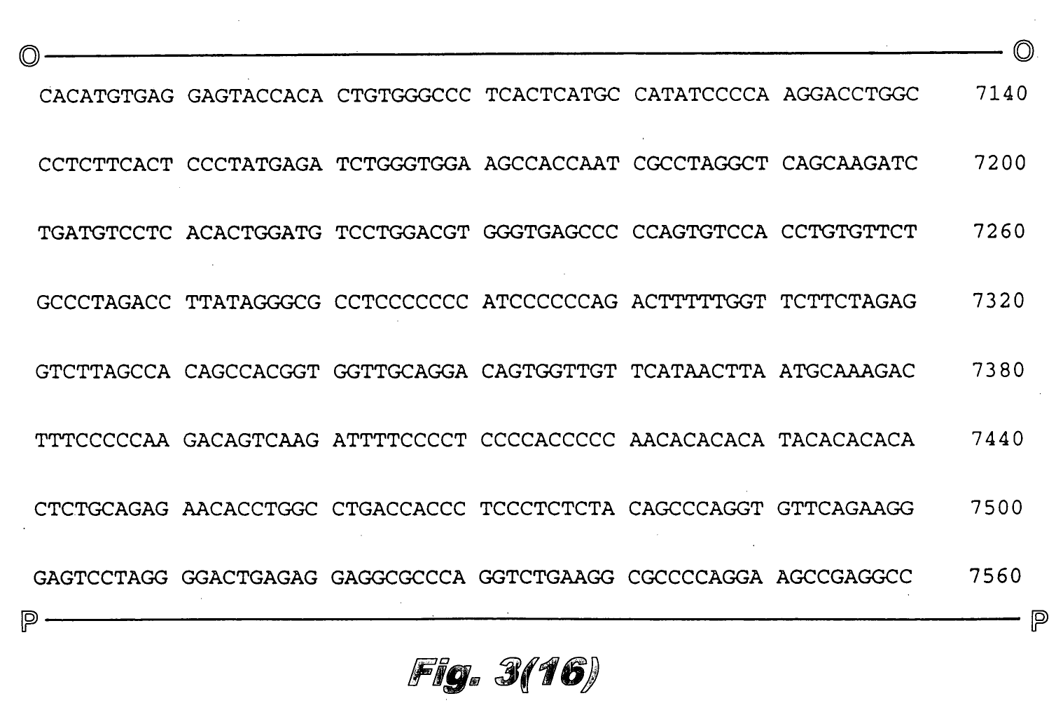

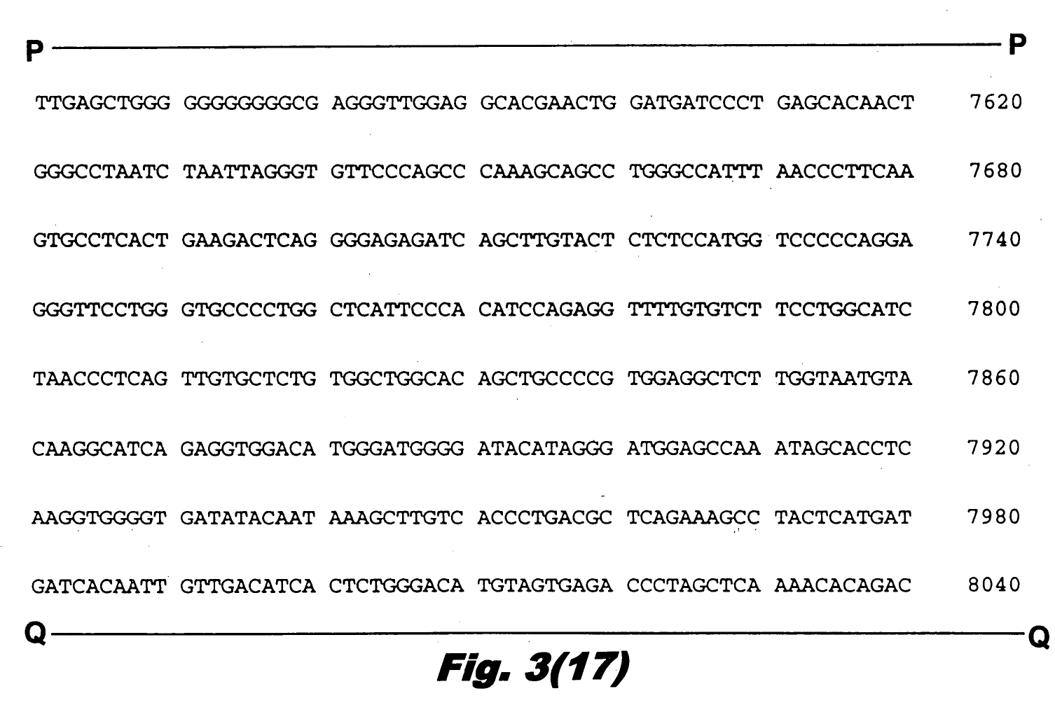

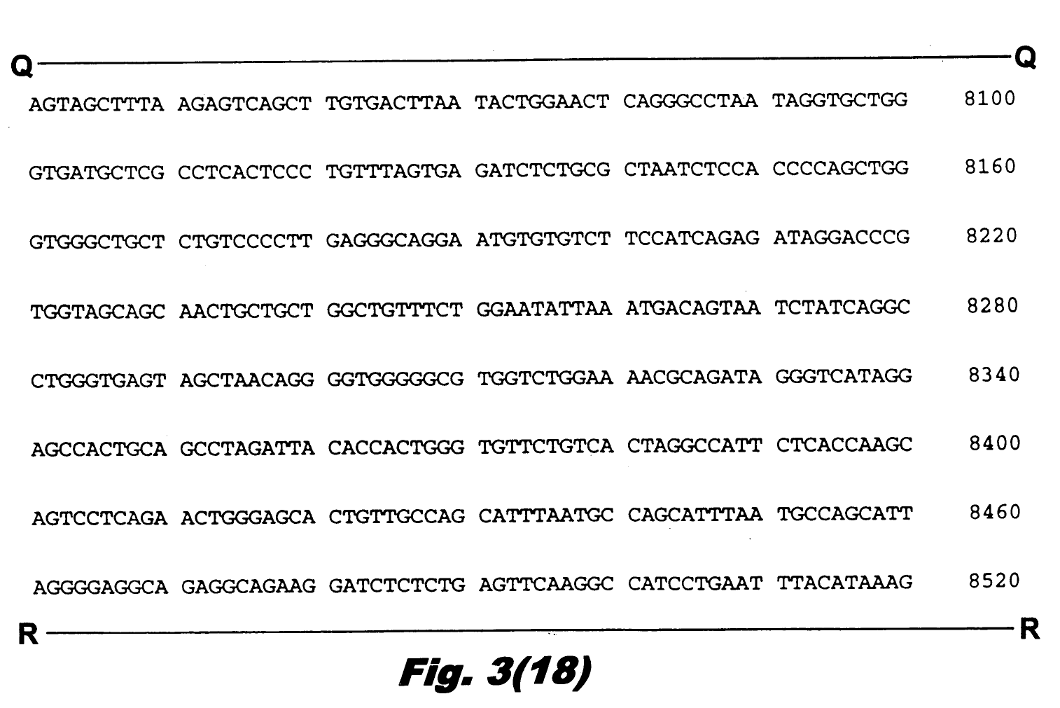

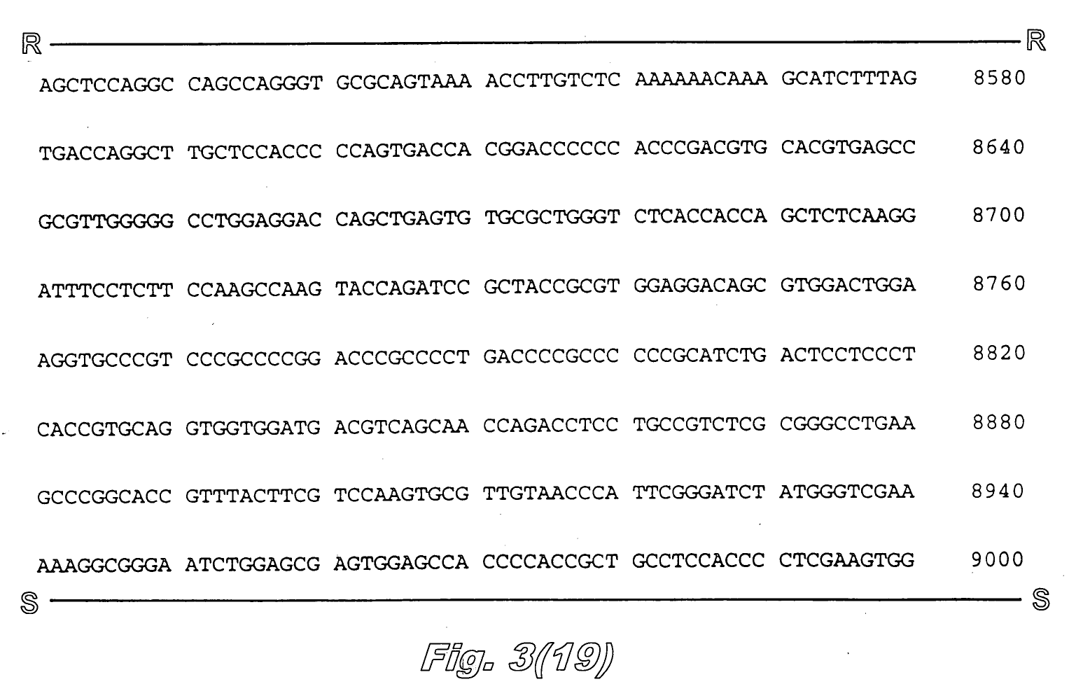

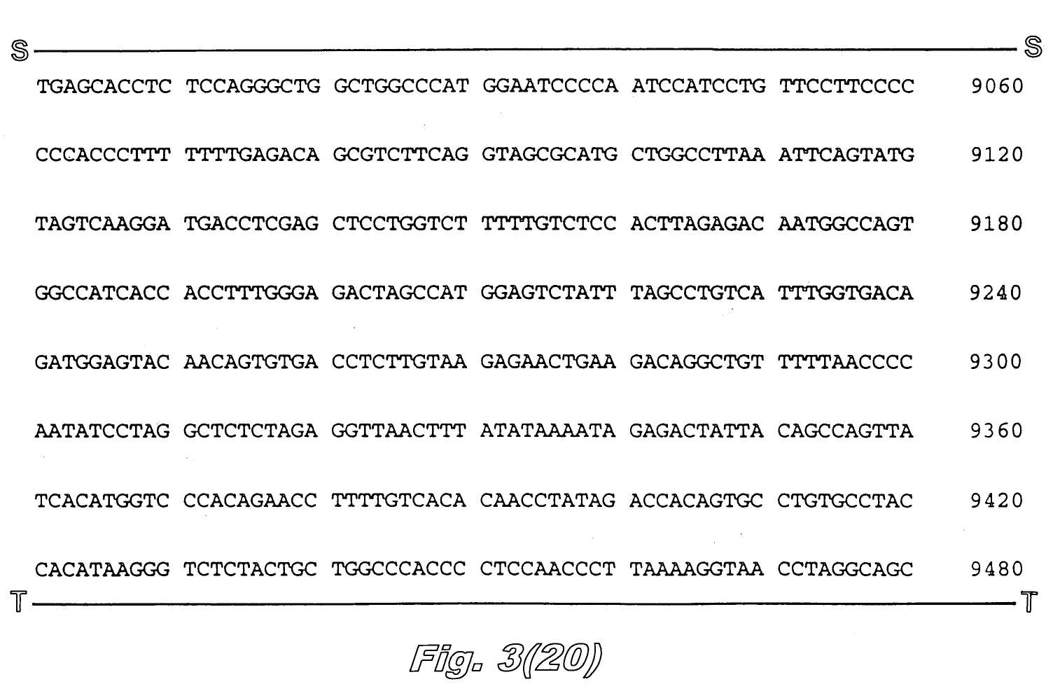

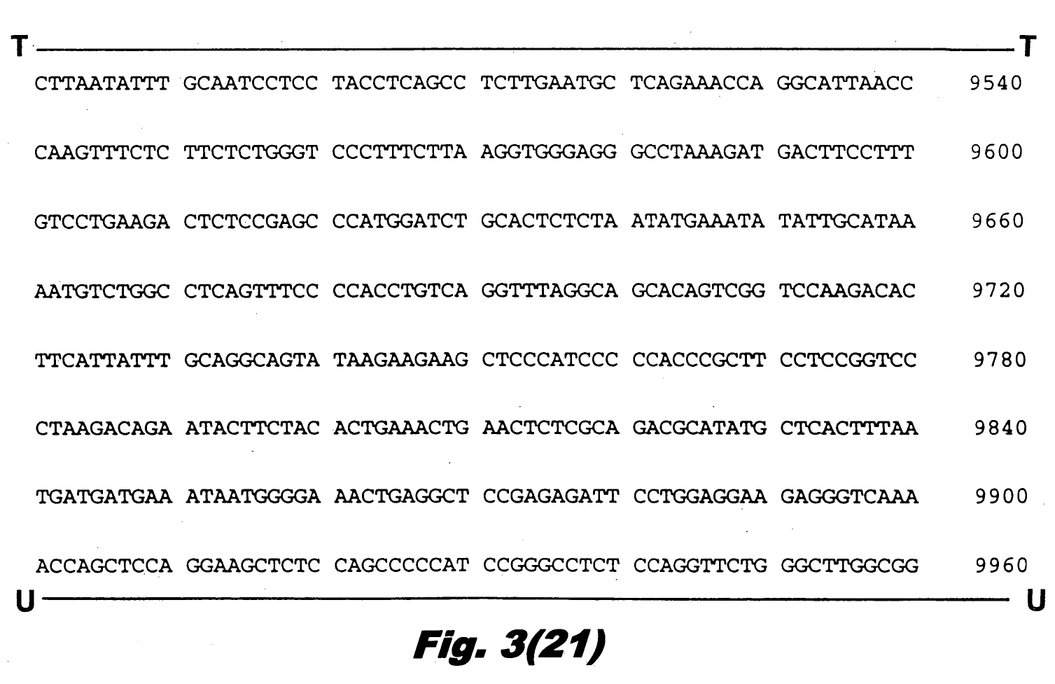

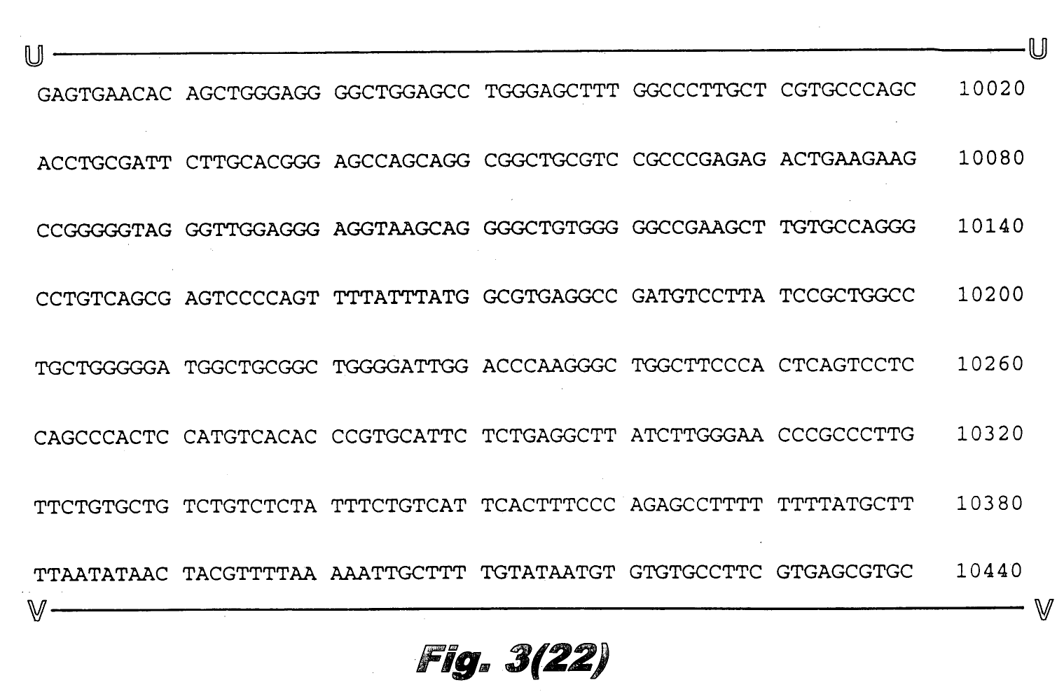

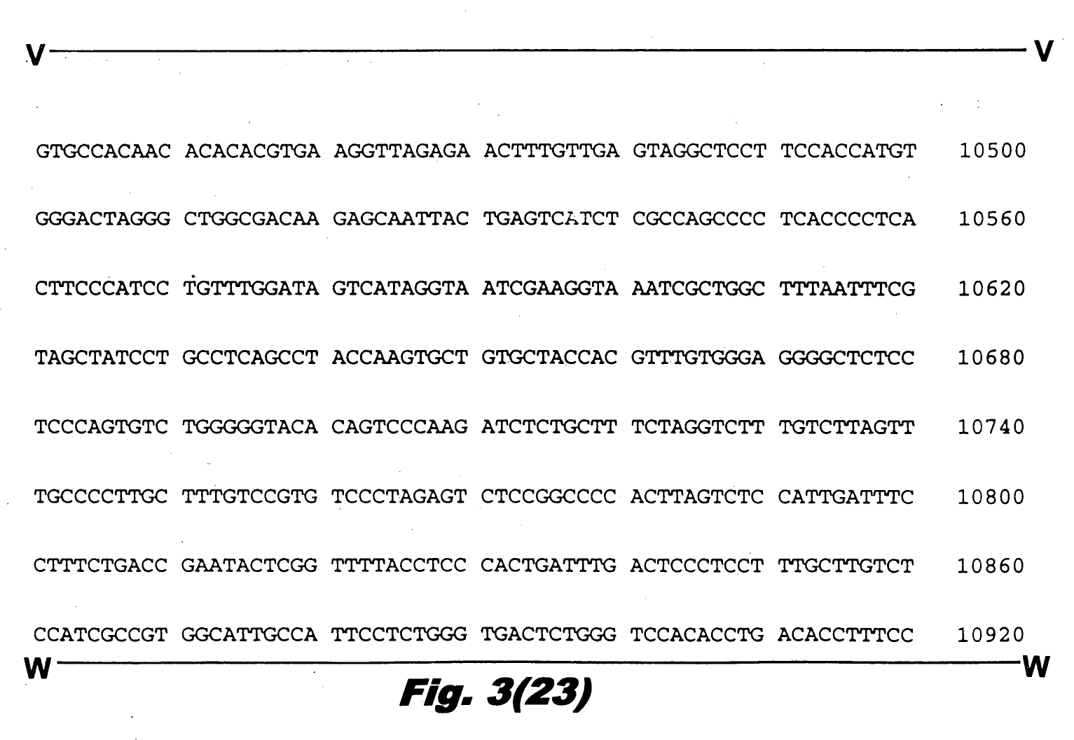

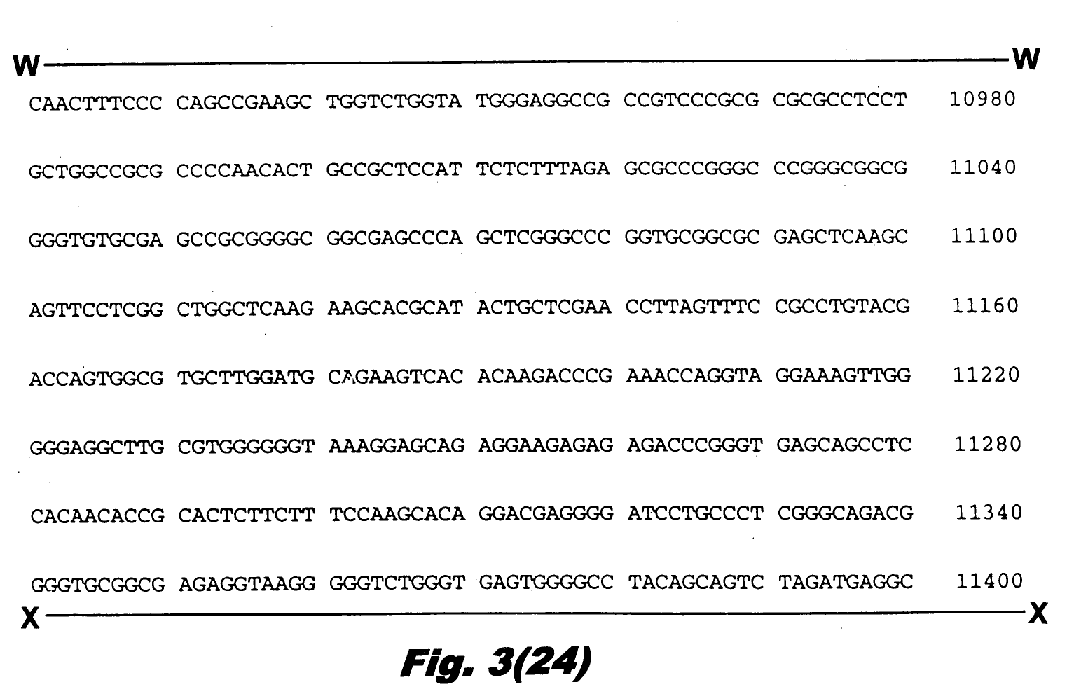

DNA encoding the murine NR6 genomic locus was also isolated using the 68-1 cDNA as a probe. Two positive clones, 2-2 and 57-3, were isolated from a mouse 129/Sv strain genomic DNA library cloned into λ FIX. These clones were overlapping and the position of the restriction sites, introns and exons were determined in the conventional manner. The region of the genomic clones containing exons and the intervening introns were sequenced on both strands using an Applied Biosystems automated DNA sequencer, with fluorescent dideoxynucleotide analogues according to the manufacturer's instructions. FIGS. 2(1)-2(9) shows the nucleotide sequence and corresponding amino acid sequence of the translation regions. This is also shown in SEQ ID NOs:30 and 31. FIGS. 3(1)-3(25) provides the genomic NR6 gene sequence but with additional 5′ sequence. This is also represented in SEQ ID NO:38 in relation to this sequence. The coding exons of NR6 span approximately 11 kb of the mouse genome. There are 9 coding exons separated by 8 introns:

| exon 1 | at least 239 | nt | intron 1 | 5195 nt |
|---|---|---|---|---|
| exon 2 | 282 | nt | intron 2 | 214 nt |
| exon 3 | 130 | nt | intron 3 | 107 nt |
| exon 4 | 170 | nt | intron 4 | 1372 nt |
| exon 5 | 158 | nt | intron 5 | 68 nt |
| exon 6 | 169 | nt | intron 6 | 2020 nt |
| exon 7 | 188 | nt | intron 7 | 104 nt |
| exon 8 | 43 | nt | intron 8 | 181 nt |
| exon 9 | 252 | nt | | |

Exon 1 encodes the signal sequence, exon 2 the Ig-like domain, exons 3 to 6 the hemopoietin domain. Exons 7, 8 and 9 are alternatively spliced.

### EXAMPLE 5

### 5′ RACE Analysis of NR6

5′-RACE was used to investigate the nature of the sequence 5′ of nucleotide 960, encoding Ile321 of NR6.1, 2 and 3. The nucleotide and corresponding amino acid sequences are shown in SEQ ID NOs: 12, 14 and 16, respectively. 5′-RACE was performed using Advantage KlenTaq polymerase (CLONTECH, CAT NO. K1905-1) on mouse brain Marathon-ready cDNA (CLONTECH, CAT NO. 7450-1) according to the manufacturer's instructions. Briefly, the first rounds of amplification were performed using 5 μl of cDNA in a total volume of 50%, with 1 mM each of the primers AP1&M116 [SEQ ID NO:2] or AP1&M159 [SEQ ID NO:4] by 35 cycles of 94° C.×0.5 min, 68° C.×2.0 min on GeneAmp 2400 (Perkin-Elmer). An amount of 5 μl of 50-fold diluted product from the first amplification was then re-amplified; for the products generated with primers AP1 and M116 [SEQ ID NO:2] in the first amplification, 1 mM of the primers AP2&M108 [SEQ ID NO:3] were used in the second amplification. For the products generated with primers AP1 and M116 [SEQ ID NO:2] in the first amplification, two separate secondary reactions were performed, one reaction with 1 mM primers AP2&M242 [SEQ ID NO:5] and the other with 1 mM primers AP2&M112 [SEQ ID NO:6]. Amplification was achieved using 25 cycles of 94° C.×0.5 min, 68° C.×2.0 min. These samples were analyzed by agarose gel electrophoresis. When a single ethidium bromide staining amplification product was observed, it was purified by QlAquick PCR purification kit according to the manufacturer's instructions (QIAGEN, CAT NO. DG-0281) and its sequence was directly determined using both primers used in the secondary amplification step, that is AP2 and either M108 [SEQ ID NO:3], M242 [SEQ ID NO:5] or M112 [SEQ ID NO:6].

### EXAMPLE 6

### Cloning of NR6

From the initial screens of mouse brain and testis cDNA libraries with the degenerate WSXWS oligonucleotides and subsequent screening of cDNA libraries from mouse testis, mouse brain and the KUSA osteoblastic cells line a total of 18 NR6 cDNAs have been isolated. Nucleotide sequence of NR6 was also determined from 5′RACE analysis of brain cDNA. Additionally, two murine genomic DNA clones encoding NR6 have also been isolated.

Comparison of the NR6 cDNA clones revealed a common region of nucleotide sequence which included a 123 base pairs 5′-untranslated region and 1221 base pairs open reading frame, stretching from the putative initiation methionine, Met1 to Gln407 (SEQ ID NOs:12, 14 and 16, respectively). Within this common open reading frame, a haemopoietin receptor domain was observed which contained the four conserved cysteine residues and the five amino acid motif WSXWS typical of members of the haemopoietin receptor family, was observed.

Further analyses revealed that after nucleotide 1221, three different classes of NR6 cDNAs could be found, these were termed NR6.1, NR6.2 and NR6.3 (SEQ ID NOs: 12, 14 and 16, respectively). Each encoded a receptor that appeared to lack a classical transmembrane domain and, would, therefore be likely to be secreted into the extracellular environment. Although the putative C-terminal region of the three classes of NR6 proteins appear to be different, the cDNAs encoding them also had a common region of 3′-untranslated region.

With regard to SEQ ID NOs:12, 14 and 16, the number of both nucleotides and amino acids begins at the putative initiation methione. NR6.1 and NR6.2 are identical to nucleotide 1223 encoding Q407, this represents the end of an exon.

NR6.1 splices out an exon present only in NR6.2 and uses a different reading frame for the final exon which is shared with NR6.2. The 3′-untranslated region is shared by NR6.1, NR6.2 and NR6.3, NR6.2 splices in an exon starting with nucleotide 1224 encoding D408 and ending with nucleotide 1264 encoding the first nucleotide in the codon for G422 and uses a different reading frame for the final exon which is shared with NR6.2 (see FIGS. 1A-1B). NR6.3 fails to splice from position nucleotide 1224, therefore, translation continues through the intron, giving rise to the C-terminal protein region.

The sequence of NR6 cDNA products generated by 5′-RACE amplification from mouse brain cDNA preparation is shown in SEQ ID NO: 18. The nucleotide sequence identified using 5′-RACE appeared to be identical to the sequence of cDNAs encoding NR6.1, NR6.2, and NR6.3 from nucleotide C151, the first nucleotide for the codon for Pro51. 5′ of this nucleotide, the sequences diverged and the sequence is unique not being found in NR6.1, NR6.2 or NR6.3. Additionally, there is a single nucleotide difference, with the sequence from the RACE containing an G rather than an A at nucleotide 475, resulting in Thr159 becoming Ala.

Analysis of the genomic clones, revealed that they were overlapping and contained exons encoding the majority of the coding region of the three forms of NR6 (FIGS. 1, 2 and 3). These genomic clones, contained exons encoding from Asp50 (nucleotide 148) of the NR6 cDNAs. Sequence 5′ of this in the cDNAs, including the 5′-untranslated region and the region encoding Met1 to Gln49 (SEQ ID NOs:12, 14 and 16), and the 5′ end predicted from analysis of 5′ RACE products (SEQ ID NO: 18) were not present in the two genomic clones isolated.

Analysis of the NR6 genomic DNA clones also provided an explanation of the three classes of NR6 cDNAs found. It is likely that NR6.1, NR6.2 and NR6.3 arise through alternative splicing of NR6 mRNA (FIGS. 1A-1B). The last amino acid residue that these different NR6 proteins are predicted to share is Gln407. SEQ ID NO:18 shows that Gln407 is the last amino acid encoded by the exon that covers nucleotides g5850 to g6037 (see FIGS. 2(1)-2(9)). Alternative splicing from the end of this exon (FIGS. 1A-1B) accounts for the generation of cDNAs encoding NR6.1 (SEQ ID NO: 12), NR6.2 (SEQ ID NO: 14) and NR6.3 (SEQ ID NO:16). In the case of NR6.1, the region from g6038 to g6425 is spliced out, leading to juxtaposition of g6037 and g6426. In the case of NR6.2, the region from g6038 to 6141 is spliced out, an exon from 6142 to g6183 is retained and then this is followed by splicing out of the region from g6183 to g6425. NR6.3 appears to arise when there is no splicing from nucleotide g6038. For all three forms, a secreted rather then transmembrane form is generated, these differ however in their predicted C-terminal region. The genomic NR6 sequence with additional 5′ sequence is shown in FIGS. 3(1)-3(25).

### EXAMPLE 7

### ESTs

Databases were searched with the murine NR6 corresponding to the unspliced version shown in SEQ ID NO:16. The murine NR6 sequence used is shown in SEQ ID NO:22.

The databases searched were:

(i) dbEST—Database of Expressed Sequence Tags National Center for Biotechnology Information National Library of Medicine, 38A, 8N8058600 Rockville Pike, Bethesda, Md. 20894 Phone: 0011-1-301-496-2475 Fax: 0015-1-301-480-9241 USA.

(ii) DNA Data Bank of Japan DNA Database Release 3689. Prepared by: Sanzo Miyazawa Manager/Database Administrator HidenoriHayashida Scientific Reviewer Yukiko Yamazaki/Eriko Hatada/Hiroaki Serizawa Annotators/reviewers Motono Horie/Shigeko Suzuki/Yumiko SataoSecretaries/typists DNA Data Bank of JapanNational Institute of Genetics Center for Genetic Information research Laboratory of Genetic Information Analyses 1111 YataMishima, Shizuoka 411 Japan.

(iii) EMBL Nucleic Acid Sequence Data Bank Release 47.0.

(iv) EMBL Nucleic Acid Sequence Data Bank Weekly Updates Since Release 44.

(v) Genetic Sequence Data Bank NCBI-GenBank Release 94 National Center for Biotechnology Information National Library of Medicine, 38A, 8N₈O₅ 8600 Rockville Pike, Bethesda, Md. 20894 Phone: 0011-1-301-495-2475 Fax: 0015-1-301-480-9241 USA.

(vi) Cumulative Updates since NCBI-GenBank Release 88 National Center for Biotechnology Information National Library of Medicine, 38A, 8N₈O₅ 8600 Rockville Pike, Bethesda, Md. 20894 USA.

The search of the databases with the murine probe identified several EST's having sequence similarity to the probe. The EST's were:

W66776 (murine sequence)

MM5839 (murine sequence)

AA014965 (murine sequence)

W46604 (human sequence)

W46603 (human sequence)

H14009 (human sequence)

N78873 (human sequence)

R87407 (human sequence).

### EXAMPLE 8

### Isolation of 3′ cDNA Clones Encoding Human NR6

PCR products encoding human NR6 were generated using oligonucleotides UP1 and LP1 (see below) based on human ESTs (Genbank Acc:H14009, Genbank Acc:AA042914) that were identified from databases searched with murine NR6 sequence (SEQ ID NO:22). PCR was performed on a human fetal liver cDNA library (Marathon ready cDNA CLONTECH #7403-1) using Advantage Klen Taq Polymerase mix (CLONTECH #8417-1) in the buffer supplied at 94° C. fro 30 s and 68° C. for 3 min for 35 cycles followed by 68° C. for 4 min and then stopping at 15° C. A standard PCR programme for the Perkin-Elmer GeneAmp PCT system 2400 thermal cycle was used. The PCR yielded a prominent product of approximately 560 base pairs (bp; SEQ ID NO:18), which was radiolabelled with [α-³²P] dCTP using a random priming method (Amersham, RPN, 1607, Mega prime kit) and used to screen a human fetal kidney 5′-STRETCH PLUS cDNA library (CLONTECH #HL1150x). Library screens were performed using Rapid Hybridisation Buffer (Amersham, RPn 1636) according to manufacturer's instructions and membranes washed at 65° C. for 30 min in 0.1×SSC/0.1% (w/v) SDS. Two independent cDNA clones were obtained as lambda phage and subsequently subcloned and sequenced. Both clones (HFK-63 and HFK-66) contained 1.4 kilobase (kb) inserts that showed sequence similarity with murine NR6. The sequence and corresponding amino acid translation of HFK-66 is shown in SEQ ID NO:24.

The translation protein sequences of clone HFK-66 shows a high degree of sequence similarity with the mouse NR6.

Oligonucleotides

| | | |
|---|---|---|
| UP1: | 5′TCC AGG CAG CGG TCG GGG GAC | [SEQ ID NO: 26] |
| | AAC 3′ | |
| LP1: | 5′TTG CTC ACA TCG TCC ACC ACC | [SEQ ID NO: 27] |
| | TTC 3′ | |

### EXAMPLE 9

### Genomic Structure of Human NR6

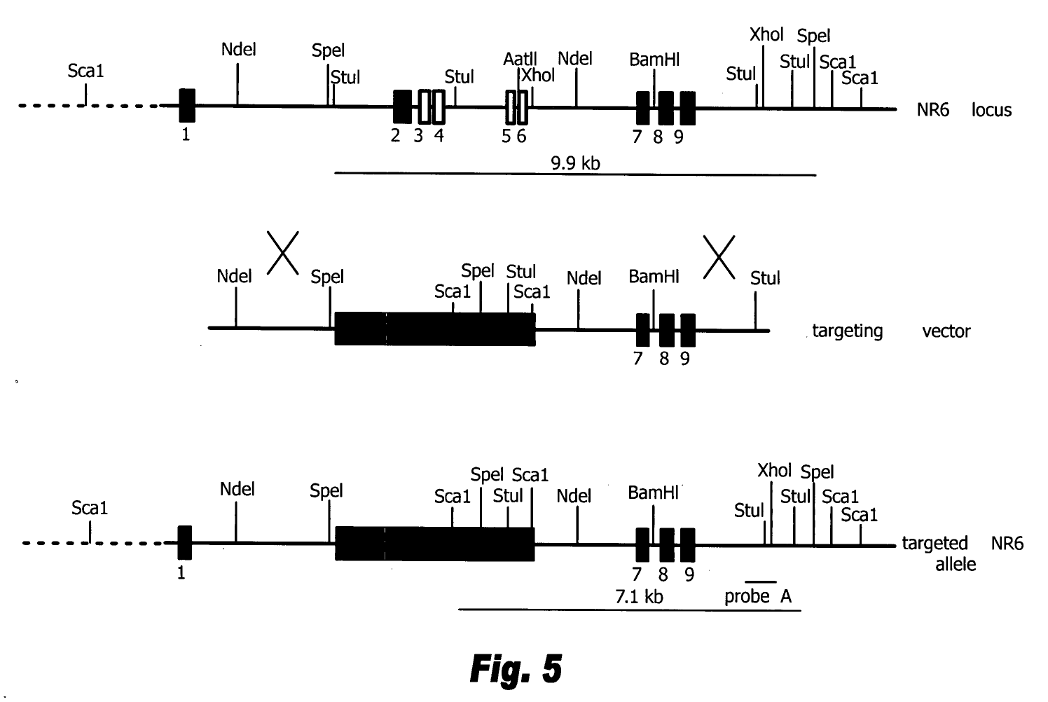

Human genomic DNA clones encoding human NR6 was isoloated by screening a human genomic library (Lambda FIX™II Stratagene 946203) with radiolabelled oligonucleotides, 2199 and 2200 (see below). These oligonucleotides were designed based on human ESTs (Genbank Acc:R87407, Genbank Acc:H14009) that were identified from databases searched with murine NR6. Filters were hybridised overnight at 37° C. in 6×SSC containing 2 mg/ml bovine serum albumin, 2 mg/ml Ficoll, 2 mg/ml polyvinylpyrrolidone, 100 mM ATP, 10 mg/ml tRNA, 2 mM sodium pyrophosphate, 2 mg/ml salmon sperm DNA, 0.1% (w/v) SDS and 200 mg/ml sodium azide and washed at 65° C. in 6×SSC/0.1% SDS. Five independent genomic clones were obtained and sequenced. The extend of sequence obtained has determined that the clones overlap and exhibit a similar genomic structure to murine NR6. Exon coding regions are almost identical over the region covered by the genomic clones while intron coding regions differ, although the size of the introns are comparable. The extent of known overlap is shown in FIG. 5.

Oligonucleotides:

| | | |
|---|---|---|
| 2199: | 5′ CCC ACG CTT CTC ATC GGA | [SEQ ID NO: 36] |
| | TTC TCC CTG 3′ | |
| 2200: | 5′ CAG TCC ACA CTG TCC TCC | [SEQ ID NO: 37] |
| | ACT CGG TAG 3′ | |

### EXAMPLE 10

### Northern Blot Analysis of Human NR6 mRNA Expression

Clontech Multiple Tissue Northern Blots (Human MTN Blot, CLONTECH #7760-1, Human MTN Blot IV, CLONTECH #7766-I, Human Brain MTN Blot II, CLONTECH #7755-1, Human Brain MTN Blot III, CLONTECH #7750) were probed with a radiolabelled 3′ human NR6 cDNA clone, HFK-66 (SEQ ID NO:24). The clone was labelled with [α-³²P] dCTP using a random priming method (Amersham, RPN 1607, Mega prime kit). Hybridisation was performed in Express Hybridisation Solution (CLONTECH H50910) for 3 hours at 67° C. and membranes were washed in 0.1×SSC/0.1% w/v SDS at 50° C.

A 1.8 kb transcript was detected in a variety of human tissues encompassing reproductive, digestive and neural tissues. High levels were observed in the heart, placenta, skeletal muscle, prostate and various areas of the brain, lower levels were observed in the testis, uterus, small intestine and colon. Photographs showing these Northern blots are available upon request. This expression pattern differs from the expression pattern observed with murine NR6.

### EXAMPLE 11

### Mouse NR6 Expression Vectors

pEF-FLAG/mNR6.1

The mature coding region of mouse NR6.1 was amplified using the PCR to introduce an in-frame Asc I restriction enzyme site at the 5′ end of the mature coding region and an Mlu I site at the 3′ end, using the following oligonucleotides:—

[SEQ ID NO: 30]

| | |
|---|---|
| 5′ oligo | 5′-AGCTGGCGCGCCTCCCGGGCGGATCGGGAGCCCAC-3′ |

[SEQ ID NO: 31]

| | |
|---|---|
| 3′ oligo | 5′-AGCTACGCGTTTAGAGTTTAGCCGGCAG-3′ |

The resulting PCR derived DNA fragment was then digested with Asc I and Mlu I and cloned into the Mlu I site of pEF-FLAG. Expression of NR6 is under the control of the polypeptide chain elongation factor 1α promoter as described (16) and results in the secretion, using the IL3 signal sequence from pEF-FLAG, of N-terminal FLAG-tagged NR6 protein.

pEF-FLAG was generated by modifying the expression vector pEF-BOS as follows:—

pEF-BOS (16) was digested with Xba I and a linker was synthesized that encoded the mouse IL3 signal sequence (MVLASSTTSIHTMLLLLLMLFHLGLQASIS)(SEQ ID NO: 32) and the FLAG epitope (DYKDDDDK)(SEQ ID NO: 55). Asc I and Mlu I restriction enzyme sites were also introduced as cloning sites.

The sequence of the linker is as follows:—

| | | |
|---|---|---|
| | M V L A S S T T S I H T M | |
| 5′ | CTAGACTAGTGCTGACACAATGGTTCTTGCCAGCTCTACCACCAGCATCCACACCATG | |
| 3′ | TGATCACGACTGTGTTACCAAGAACGGTCGAGATGGTGGTCGTAGGTGTGGTAC | |
| | L L L L L M L F H L G L Q A S I S | (SEQ ID NO: 32) |
| | Asc I | |
| | CTGCTCCTGCTCCTGATGCTCTTCCACCTGGGACTCCAAGCTTCAATCTCGGCGCGCC | |
| | GACGAGGACGAGGACTAGCAGAAGGTGGACCCTGAGGTTCGAAGTTAGAGCCGCGCGG | |
| | D Y K D D D D K | (SEQ ID NO: 55) |
| | Mlu I | |
| | AGGACTACAAGGACGACGATGACAAGACGCGTGCTAGCACTAGT 3′ | (SEQ ID NO: 56) |
| | TCCTGATGTTCCTGCTGCTACTGTTCTGCGCACGATCGTGATCAGATC 5′ | (SEQ ID NO: 57) |

The two oligonucleotides (SEQ ID NOS: 56 and 57) were annealed together and ligated into the Xba I site of pEF-BOS to give pEF-FLAG.

pCOS1/FLAG/mNR6 & pCHO1/FLAG/mNR6

A DNA fragment containing the sequences encoding IL3 signal sequence/Flag/mNR6 and the poly(A) adenylation signal from human G-CSF cDNA, was excised from pEF-FLAG/mNR6 using the restriction enzyme EcoR I. This DNA fragment was then inserted into the EcoR I cloning site of pCOS1 and pCHO1

The pCOSI and pCHO1 vectors were constructed as follows. pCH01 is also described in reference (17) but with a different selectable marker.

pCOS1 was prepared by digesting HEF-12h-gα1 (see FIG. 24 of International Patent Publication No. WO 92/19759) with EcoRI and SmaI and ligating the digesting product iwht an EcoRI-NotI-BamHI adaptor (Takara 4510). The resulting plasmid comprises an EFIα promoter/enhancer, Ncor marker gene, SV40E, ori and an Amp^{r} marker gene.

pCH01 was constructed by digesting DHFR-PMh-gr1 (see FIG. 25 of International Patent Publication No. WO 92/19759) with PvuI and Eco47III and ligating same with pCOSI digested with PvuI and Eco47III. The resulting vector, pCH01, comprises an EFIa promoter/enhancer, an DHFR marker gene, SV40E, Ori and a Amp^{r} gene.

### EXAMPLE 12

mRN6 has been expressed as an N′Flag tagged protein following transfection of CHO cells and as a C′ Flag tagged protein following transfection of KUSA cells in both cases varying levels of dimeric and aggregated NR6 were secreted.

### EXAMPLE 13

### Murine NR6 Expression

NR6 expression studies were conducted in murine Northern Blots. At the level of sensitivity used in the adult mouse, NR6 expression was detected in salivary gland, lung and testis. During embryonic development, NR6 is expressed in fetal tissues from day 10 of gestation through to birth. In cell lines, NR6 expression has been observed in the T-lymphoid line CTLL-2 as well as in FD-PyMT (FDC-P1 myeloid cells expressing polyoma midle T gene), and fibroblastoid cells including bone marrow and fetal liver stromal lines.

### EXAMPLE 14

### Expression, Purification and Characterisation of CHO and KUSA mNR6

The methods provide for the production of a dimeric form of CHO derived N′ FLAG-mNR6 without refolding. All other methods are capable of producing NR6 and are encompassed by the present invention.

A. Production of CHO Derived N′ FLAGmNR6 (Dimeric Form)

(i) Protein Production

To analyse structure and functional activity, a cDNA fragment containing the entire coding sequence of murine NR6 with an N-terminal FLAG (N′ FLAG) sequence was cloned into the EcoRI site of the expression vector pCHO1. For stable production of N-terminal FLAG-tagged NR6 the vector contains the DHFR (dihydrofolate reductase) gene as a selective marker with the NR6 gene under the control of an EF1a promoter. CHO cells were transfected with the construct using a polycationic liposome transfection reagent (Lipofectamine, GibcoBRL).

(ii) Lipofectamine Transfection Method

Using six well tissue culture plates either 2×10⁵ KUSA cells in 2 ml IMDM+10% (v/v) FCS or 2×10⁵ CHO cells were cultured in 2 ml α-MEM+10% (v/v) FCS until 70% confluent. 2 μg DNA diluted in 100 μl OPTI-MEM I (Gibco BRL, USA) was mixed gently with 12 μl lipofectamine diluted in 100 μl OPTI-MEM I and incubated at room temperature for 30 min to allow DNA complex formation. DNA complexes were gently diluted in a total volume of 1 ml of OPTI-MEM I and overlaid onto washed KUSA or CHO cell monolayers. A further 1 ml IMDM+20% (v/v) FCS (KUSA cells) or 1 ml α-MEM+20% (v/v) FCS(CHO cells) was added to transfected cells after 5 hours. At 24 hours, the culture medium was replaced with fresh complete growth medium. At 48 hours after transfection, selection was applied. A methotrexate resistant clone secreting comparatively high levels of NR6 was selected and expanded for further analysis.

(iii) Protein Expression

CHO cells were grown to confluence in roller bottles in nucleoside free α-MEM+10% (v/v) FCS. Selection was maintained by using 100 ng/ml Methotrexate in the conditioned media according to manufacturer instructions. Expression was monitored by Biosensor and harvesting found to be optimal at 3 to 4 days.

B. Protein Analysis

(i) Biosensor Analysis

Expression and purification was monitored by Biosensor analysis (BiaCore™, Sweden) where anti FLAG peptide M2 antibody (Kodak Eastman, USA), specific for the FLAG peptide sequence was bound to the sensorchip. Fractions were analysed for binding to the sensor surface (resonance units) and the sample then removed from the surface using 50 mM Diethylamine pH 12.0 prior to analysis of the next fraction. Immobilisation and running conditions of the Biosensor follow the manufacturer's instructions.

(ii) Protein Production

In order to generate and characterise NR6, conditioned media (2 L) produced by CHO cells was harvested after day 3, post confluence. Conditioned media was concentrated using diafiltration with a 10,000 molecular weight cut-off. (Easy flow, Sartorius, Aus). At a volume of 200 ml (i.e. 10× concentrated) the sample was buffer exchanged into 20 mM Tris, 0.15M NaCl, 0.02% (v/v) Tween 20 pH 7.5 (Buffer A).

(iii) Immunoprecipitation and Western Blot Analysis of mNR6

Concentrated conditioned media (1 ml) was immunoprecipitated with M2 affinity resin (20 μl, Kodak Eastman). To examine the structural characterisation of mNR6 SDS PAGE was performed under reducing and non-reducing conditions. Separation was performed on NOVEX 4-20% (v/v) Tris/glycine gradient gels and protein transfered on PVDF membrane. Western blots were probed with biotinylated M2 antibody (primary, 1:500) and then streptavidin peroxidase (secondary, 1:3000). Samples were visualised by autoradiography using electrochemiluminescence (ECL, Dupont, USA).

By regressional analysis of prestained standards (BIORAD, Aus.) the molecular weight of the monomeric unit was calculated to be 65,000 daltons. Under non-reducing conditions the molecular weight was calculated to be 127,000 indicating that NR6 is a disulphide linked dimer. A tetrameric complex rnning at approximately 250,000 daltons was also observed. Although a band running at approximately 50,000 daltons was observed, no monomeric NR6 was detected under non-reducing conditions indicating that the majority of NR6 expressed in this system is disulphide linked.

(iv) Affinity Chromatography of mNR6

Concentrated conditioned media (200 ml) was applied to M2 affinity resin (5 ml) under gravity. To enhance recovery the unbound fraction was reapplied to the column four times prior to extensive washing of the column with 200 volumes of Buffer A. Biosensor analysis indicates that approximately 20% of the M2 binding originally present in the concentrate remains in the unbound fraction. The bound fraction was eluted from the column using an immunodesorbant (50 ml); actisep (Sterogene Labs, USA).

(v) Ion exchange and Desalting of mNR6

In order to buffer exchange mNR6 prior to anion chromatography, 10 ml batches of the eluted fraction (50 ml) were applied to an XK column (400×26 mm I.D.) containing G25 sepharose (Pharmacia, Sweden). Chromatography was developed at 4 ml/min using an FPLC (Pharmacia, Sweden) equipped with an online UV280 and conductivity monitor. The mobile phase was 10 mM Tris, 0.1M NaCl, 0.02% v/v Tween, pH 8.0. 10 ml fractions were collected between 12.5 min and 25 min to optimise recovery and removal of salt. Fractions were analysed by Biosensor analysis and pooled according to binding.

All pooled active fractions were diluted with an equal volume of 20 mM Tris, 0.02% (v/v) Tween, pH 8.5 (Buffer B) and then loaded onto a Mono Q 5/5 (Pharmacia, Sweden) at a flow rate of 2 ml/min. The column was washed with buffer B. Elution was performed using a linear gradient between buffer B and buffer B containing 0.6M NaCl over 30 min at a flow rate of 1 ml/min. Fractions (1 minute) were collected and analysed on the Biosensor and also by SDS PAGE and Western blot analysis. Fractions 15 to 26 (approximately 0.4M NaCl) appear to contain the majority of mNR6 as indicated by the Biosensor.

C. Production of CHO Derived N′FLAGmNR6 (Monomeric Form)

(i) Protein Production

A cDNA fragment containing the entire coding sequence of murine NR6 with an N-terminal FLAG™ sequence was cloned into the expression vector pCHO1 for production of N-terminal FLAG-tagged protein. This vector contains a neomycin resistance gene with expression of the NR6 gene under the control of an EF1α promoter. This expression construct was transfected into CHO cells using Lipofectamine (Gibco BRL, USA) according to the manufacturer instructions. Transfected cells were cultured in IMDM+10% (v/v) FCS with resistant cells selected in geneticin (600 μg/ml, Gibco BRL, USA). A neomycin resistant clone, secreting comparatively high levels of NR6 was selected and expanded for further analysis.

(ii) Protein Expression

N′FLAG-NR6 expressed in serum free conditioned media (10 litre) was harvested from transfected CHO and cells. Collected media was concentrated using a CH2 ultrafiltration system equipped with a S1Y10 cartridge (Amicion molecular weight cut-off 10,000). Preliminary examination of the expressed product under reducing and non-reducing SDS PAGE followed by western blot analysis was performed. Visualisation of the protein on Westerns was specific to the primary antibody anti FLAG M2. Under reducing conditions a band approximately at 65,000 daltons was observed. Under non-reducing conditions, dimer and larger molecular weight aggregates were observed. These are disulphide linked monomers as they are not present in the reducing gel. Small amounts of monomer appear to be present in non-reducing gels.

(iii) Affinity Chromatography of NR6

Concentrated conditioned media was applied to an anti FLAG M2 affinity resin (100×16 mm I.D.). After washing the unbound proteins off the column, the bound proteins were eluted using FLAG peptide (60 μg/ml) in PBS.

(iv) Ion Exchange Chromatography of NR6

Eluted fractions from affinity column were dialysed overnight against 20 mM Tris-HCl pH 8.5 (buffer C) containing 50 mM Dithiothretol (DTT) using 25,000 cut-off dialysis tubing (Spectra/Por7, Spectrum). The dialysed fractions were loaded onto Mono Q 5/5 (Pharmacia, Sweden) previously equilibrated with buffer C containing 5 mM DTT. Chromatography was developed using a linear gradient between buffer C and buffer C containing 1.0 M NaCl at a flow rate of 0.5 ml/min.

(v) Refolding of NR6

Fractions containing NR6 from the Mono Q were adjusted to 50 mM DTT and left overnight at 4° C. To initiated refolding the sample was then dialysed against 50 mM Tris-HCl (pH 8.5), 2 M Urea, 0.1% (v/v) Tween 20, 10 mM Glutathione (reduced) and 2 mM Glutathione (oxidised) at a final protein concentration of 100 μg/ml. Folding was carried out at ambient temperature with one change of the buffer over 24 hours.

(v) Reversed Phase High Performance Liquid Chromatography (RP-HPLC)

The folded product was further purified by RP-HPLC using a Vydac C4 resin (250×4.6 mm I.D.) previously equilibrated with 0.1% (v/v) Trifluoroacetic acid (TFA). Elution was carried out using a linear gradient from 0 to 80% (v/v) acetonitrile/0.1% (v/v) TFA at a flow rate of 1 ml per minute.

D. pCHO1/NR6/FLAG

In order to determine the native N termini of NR6, a C terminal FLAG NR6 CHO cell line was established.

The plasmid pKUSA166 (murine NR6 cDNA cloned into the EcoR I site of pBLUESCRIPT) was digested with BamH I to remove the sequences encoding the last 15 amino acids of murine NR6. Synthetic oligonucleotides which encode the 3′ end of mouse NR6 followed by the FLAG peptide tag were annealed and ligated into the BamH I site of pKUSA166. The sequence of the oligonucleotides was as follows:—

[SEQ ID NO: 34]

I L P S G R R G A A R G P A G D Y K D D D D K *

[SEQ ID NO: 33]

GATCTTGCCCTCGGGCAGACGGGGTGCGGCGAGAGGTCCTGCCGGCGAC
TACAAGGACGACGATGACAAGTA G

[SEQ ID NO: 35]

AACGGGAGCCCGTCTGCCCCACGCCGCTCTCCAGGACGGCCGCTGATGT
TCCTGCTGCTACTGTTCATCCTAG

The 5′ end of the linker introduces a silent mutation (CTG>TTG), to destroy the 5′ BamH I site upon insertion of the linker. The NR6 cDNA (with native signal sequence) with the C-terminal FLAG was cut out of pKUSA166 with EcoR I and BamH I and cloned into the EcoR I-BamH I cloning sites of pCHO-1. This vector results in the secretion of NR6 protein with a C-terminal flag tag (C′FLAG-mRN6).

This vector results in the secretion of NR6 protein from KUSA cells. The vector pCHO1 has been previously described in (17) although with a different secretable marker.

(i) Production of Polyclonal NR6 Antiserum

The following peptide from the N terminal area of NR6 was chosen for production of polyclonal antiserum to NR6

| | |
|---|---|
| VISPQDPTLLIGSSLQATCSIHGDTP | [SEQ ID NO: 39] |

The peptide was conjugated to KLH and injected into rabbits. Production and purification of the polyclonal antibody specific to the NR6 peptide sequence follows standard methods.

(ii) Protein Expression

KUSA cells transfected with cDNA of C terminal tagged mNR6 were grown to confluence in flasks (800 ml) using IMDM media containing 10% (v/v) FBS. Conditioned media (100 ml) was harvested 3-4 days post confluence.

(iii) Characterisation of NR6 by Immunoprecipitation and Western Blotting

In order to establish that NR6 with the predicted sequence is produced in KUSA cells transfected with the cDNA, western blot analysis using both M2 antibody and purified NR6 specific rabbit antibody were performed. Conditioned media (1 to 5 ml) was immunoprecipitated with M2 affinity resin (10-20 μl). Then after sufficient time for binding, the beads were washed with MT-PBS and subsequently NR6 eluted with 100 μg/ml FLAG peptide (40 μl, (1, 5 minute incubation). The sample was then subjected to reducing and non reducing SDS PAGE followed by western blot analysis. Both purified NR6 polyclonal antibody (purified by protein G) and M2 antibody recognise a band under reducing conditions of a molecular weight size approximately 65,000 daltons. Since the two antibodies reconising resides at the N terminus and C terminus it is reasonable to assume that full length NR6 is produced. Biotinylation of the respective antibodies by standard methods reduces the background. Under non-reducing conditions polyclonal NR6 bind antibodies to a band of a molecular weight of approximately 127,000, consistent with a dimeric NR6 disulphide linked form. Minor components of tetrameric NR6 are present, no monomeric NR6 is evident using polyclonal NR6 antibodies.

### EXAMPLE 15

### Generation of NR6 Knockout Mice

To construct the NR6 targeting vector, 40.1 kb of genomic NR6 DNA containing exons 2 through to 6 was deleted and replaced with G418-resistance cassette, leaving 5′ and 3′ NR6 arms of 2.9 and 4.5 kb respectively. A 4.5 kb XhoI fragment of the murine genomic NR6 clone 2.2 (FIGS. 3(1)-3(25)) containing exons 7, 8 and 3′ flanking sequence was subcloned into the XhoI site of pBluescript generating pBSNR6Xho4.5. A 2.9 kb NotI-Stul fragment within NR6 intron 1 from the same genomic clone was inserted into NotI and EcoRV digested pBSNR6Xho4.5 creating pNR6-Ex2-6. This plasmid was digested with ClaI, which was situated between the two NR6 fragments, and following blunt ending, ligated with a blunted 6 kb HindIII fragment from placZneo, which contains the laczgene and a PGKneo cassette, to generate the final targeting vector, pNR61acZneo. pNR61acZneo was linearised with NotI and electroporated into W9.5 embryonic stem cells. After 48 hours, transfected cells were selected in 175 μg/ml G418 and resistant clones picked and expanded after a further 8 days.

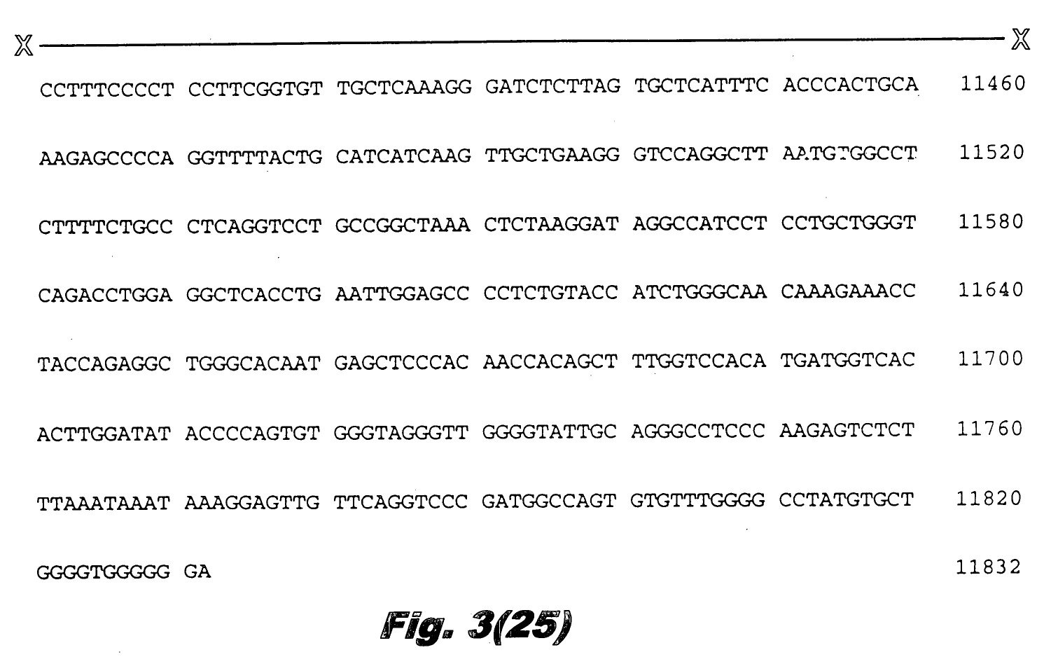

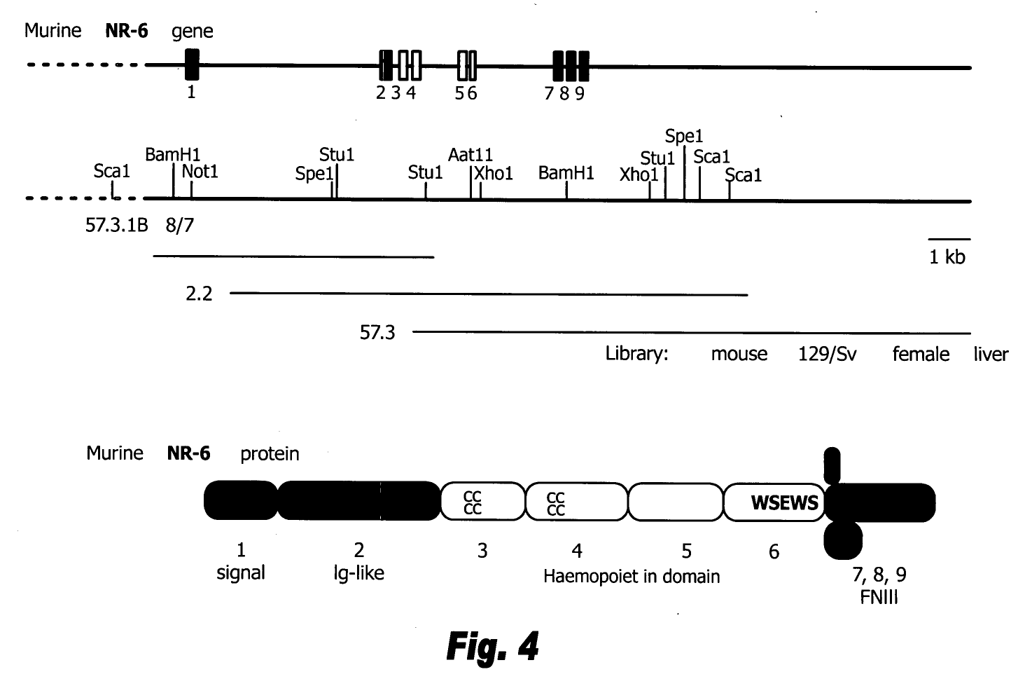

Clones in which the targetting vector had recombined with the endogenous NR6 gene were identified by hybridising SpeI-digested genomic DNA with a 0.6 kb XhoI-StuI fragment from genomic NR6 clone 2.2. This probe (probe A, FIG. 4), which is located 3′ to the NR6 sequences in the targeting vector, distinguished between the endogenous (9.9 kb) and targeted (7.1 kb) NR6 loci (FIG. 5).

Genomic DNA was digested with SpeI for 16 hrs at 37° C., electrophoresed through 0.8% (w/v) agarose, transferred to nylon membranes and hybridised to ³²P-labelled probe in a solution containing 0.5M sodium phosphate, 7% (w/v) SDS, 1 mM EDTA and washed in a solution containing 40 mM sodium posphate, 1% (w/v) SDS at 65° C. Hybridising bands were visualised by autoradiography for 16 hours at −70° C. using Kodak XAR-5 film and intensifying screens. Two targeted ES cell clones, W9.5NR6-2-44 and W9.5NR6-4-2, were injected into C57B1/6 blastocysts to generate chimeric mice. Male chimeras were mated with C57B1/6 females to yield NR6 heterozygotes which were subsequently interbred to produce wild-type (NR6^{+/+}), heterozygous (NR6^{+/-}) and mutant (NR6^{-/-}) mice. The genotypes of offspring were determined by Southern Blot analysis of genomic DNA extracted from tail biopsies.

Genotyping of mice at weaning from matings between NR^{+/-} heterozygous mice derived from both targated ES cell clones revealed an absence of homozygous NR6^{-/-} mutants. As no unusual loss of mice was observed between birth and weaning, this suggest that lack of NR6 is lethal during embryonic development or immediately after birth. Genotyping of embryonic tissues at various stages of development suggests that death occurs late in gestation (beyond day 16) or at birth.

### EXAMPLE 16

### Oligonucleotides

1943:

[SEQ ID NO: 40]

5′ GTC CAA GTG CGT TGT AAC CCA 3′
2070:

[SEQ ID NO: 41]

5′ GCT GAG TGT GCG CTG GGT CTC ACC 3′
2057:

[SEQ ID NO: 42]

5′ GGC TCC ACT CGC TCC AGA 3′

### EXAMPLE 17

### Isolation of a Full-Length Human NR6 cDNA Clones

PCR Amplification of a huNR6 Specific Probe:

Two human ESTs (Genbank Acc: AA042914 and H14009) showing homology with murine NR6 were used to design oligonucleotides for PCR screening of arange of commercially available human genomic and cDNA libraries. Oligonucleotide sequence: Fwd primer: 5′-TGC CCC CAG AGA AAC CCG TCAAC-3′ [SEQ ID NO: 45] and Rev primer: 5′-CGT GAG TAC ATC GGA GCG GGC AGA G-3′ [SEQ ID NO: 46]. The expected fiagment size of 300 bp was amplified (25 cycles, 96° C. denaturation, 60° C. annealing and 72° C. extension, Stratagene Pfu DNA polymerase Cat#600159, Corbett PC-960G) from a human placental cDNA library (Clontech Human Placenta 5′-STRETCH PLUS cDNA library Cat#HL3007b, cloning vector lgt11, oligo(dT) and random primed, source RNA25 year old Caucasian mother). PCR amplification was repeated using aproof reading polymerase (Stratagene) to generate blunt ended PCR products for cloning into pCR-Blunt vector (Invitrogen ZeroBlunt PCRCIoning Kit, Cat# 440302). PCR colony analysis was used to identify transformed E. coli containing appropriately ligated vector and theidentity of the inserts confirmed by sequencing.

Screening of Human Placental Library:

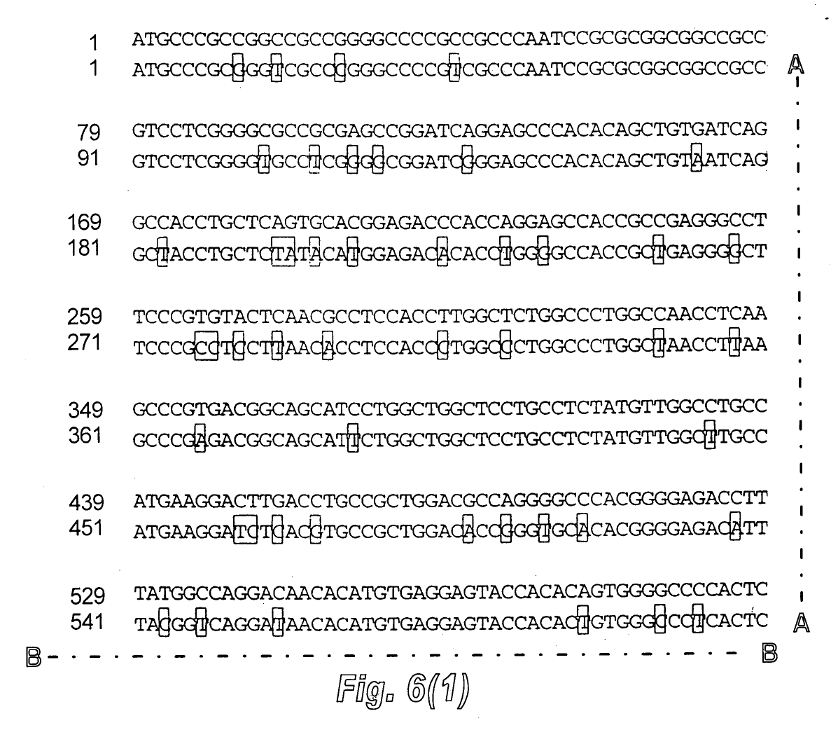

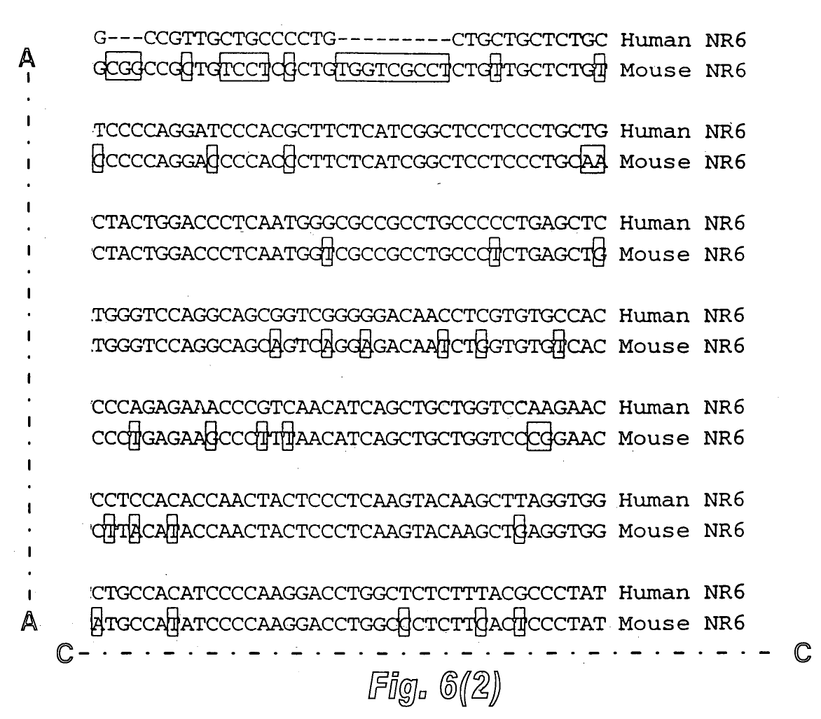

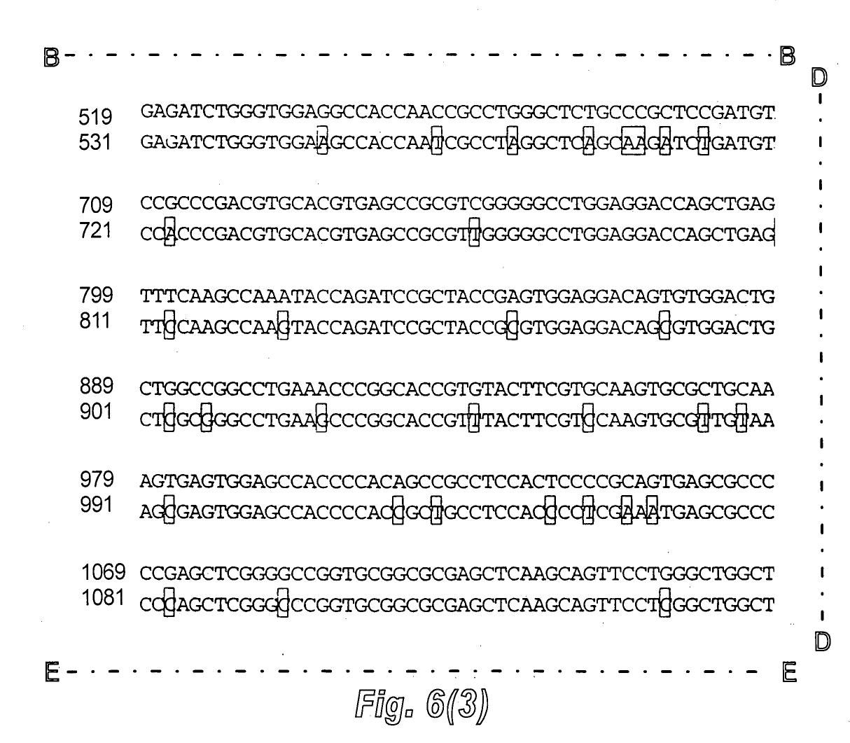

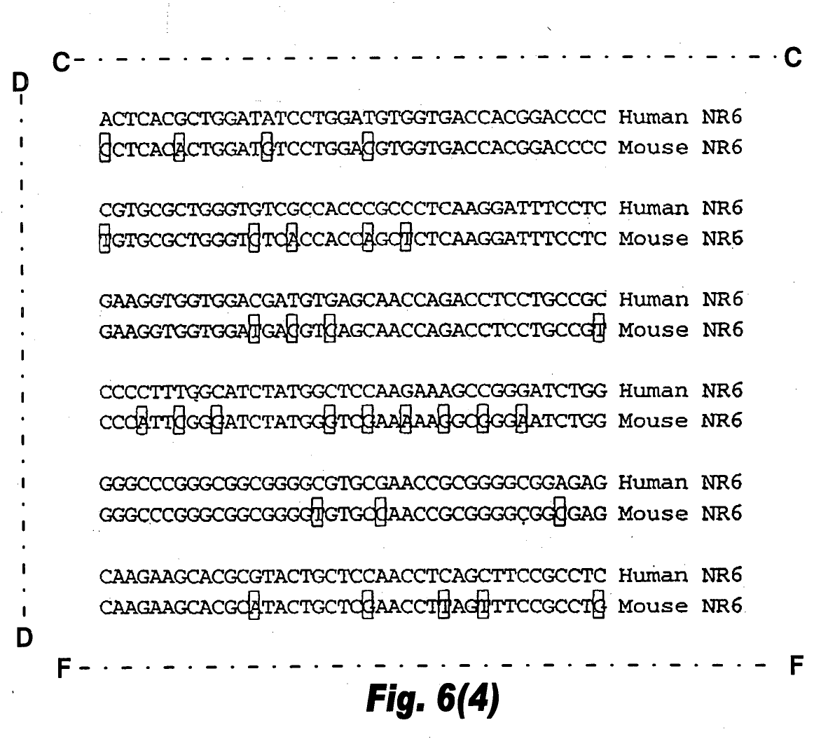

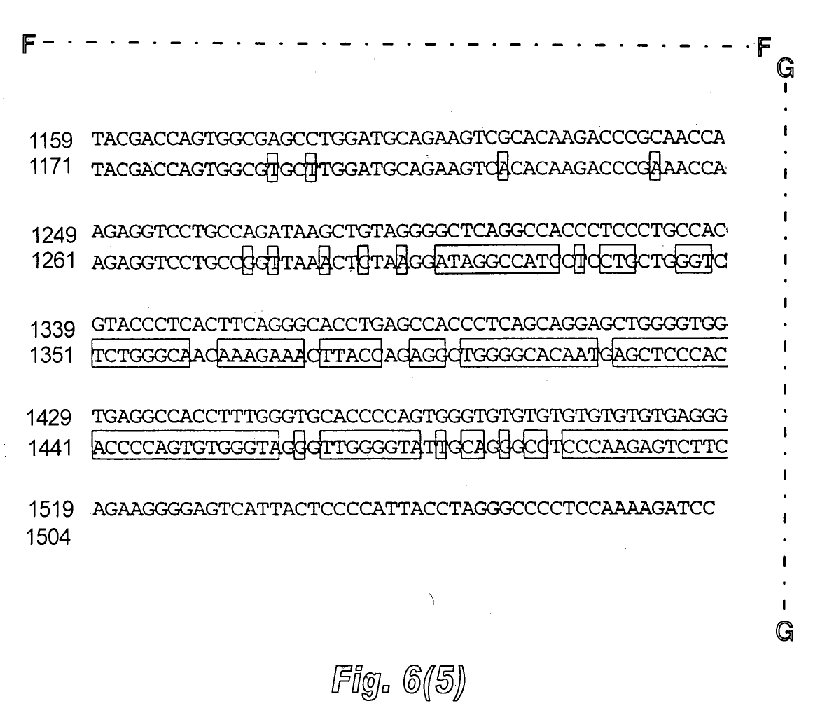

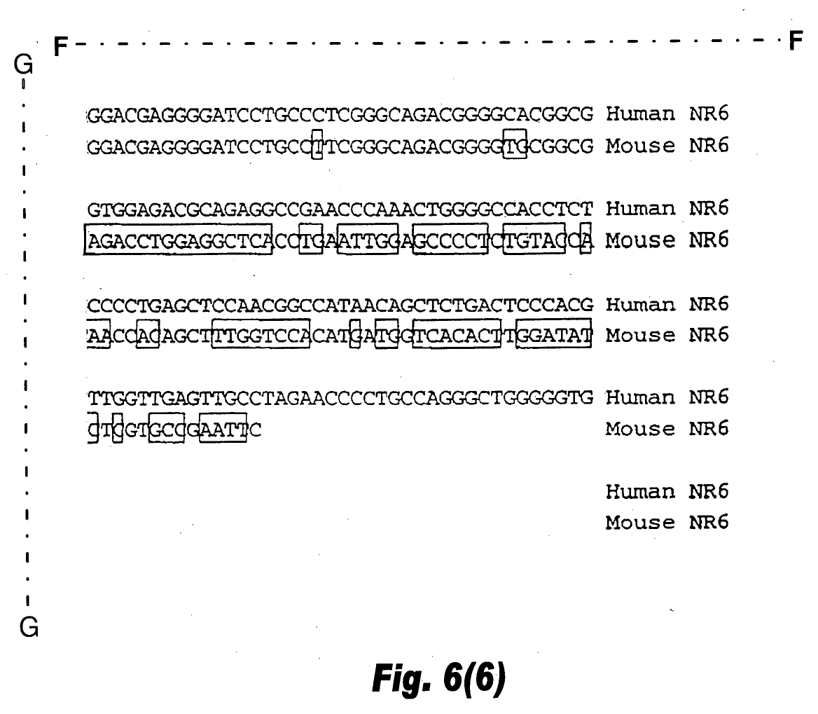

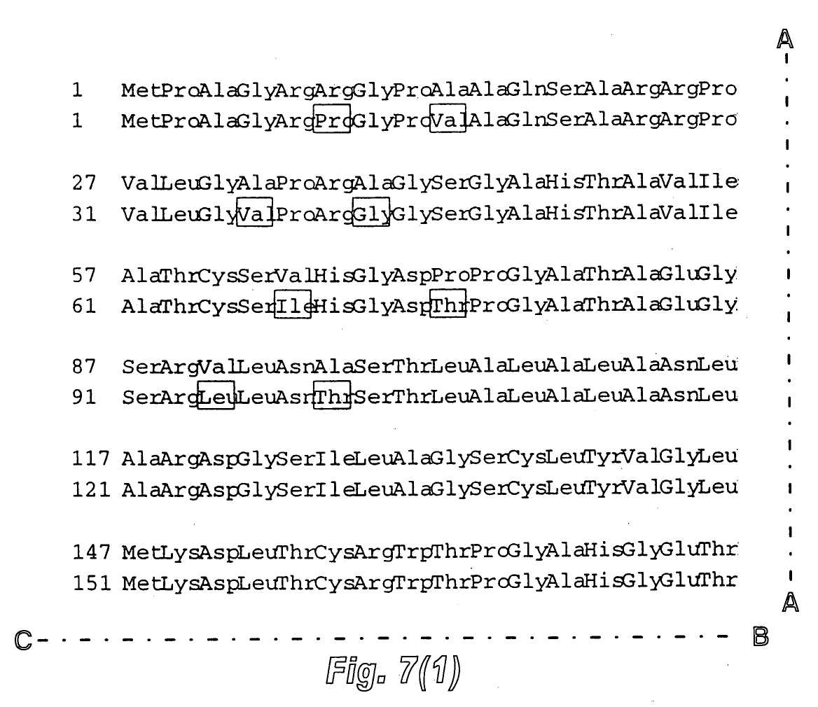

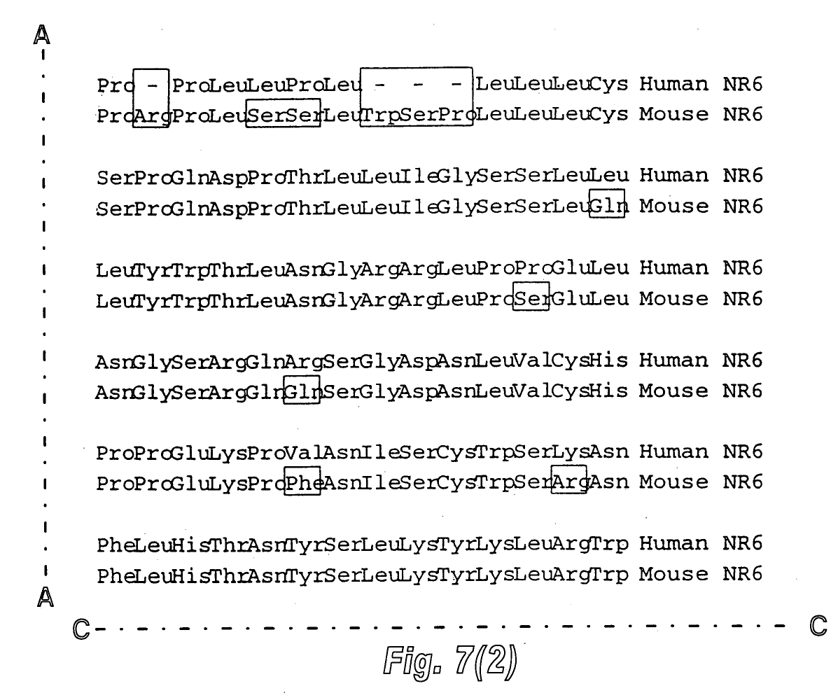

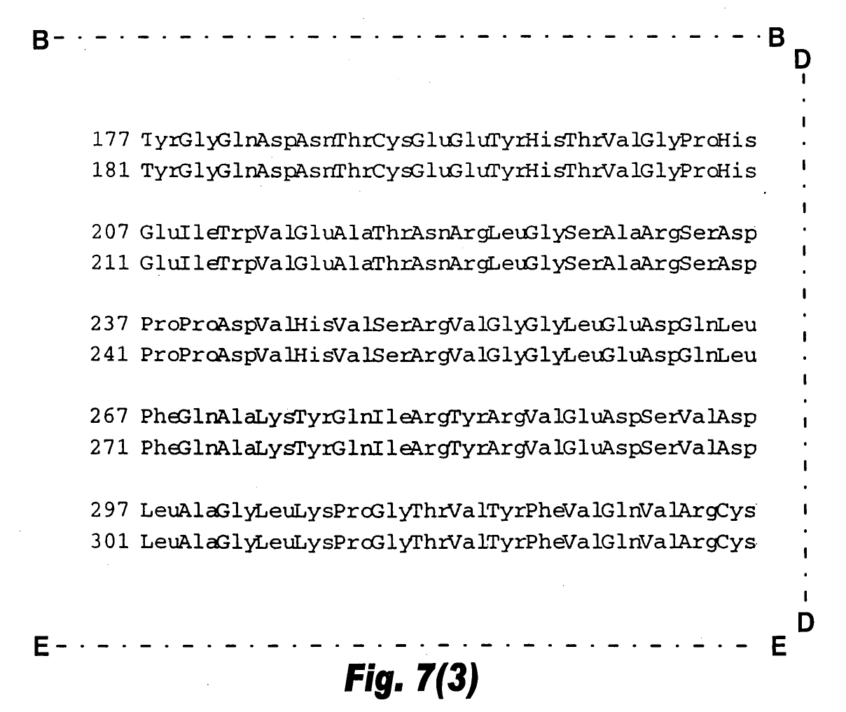

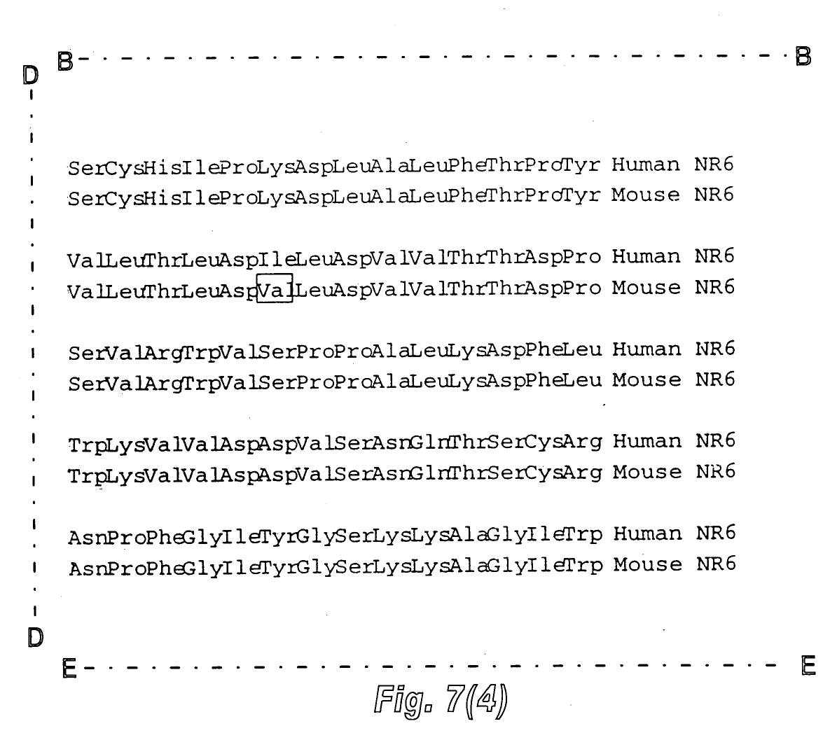

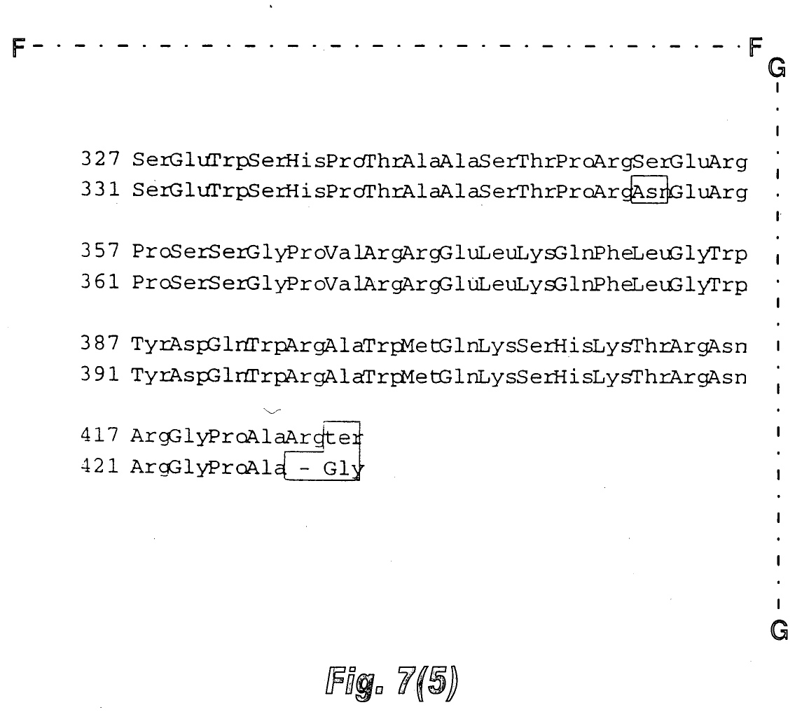

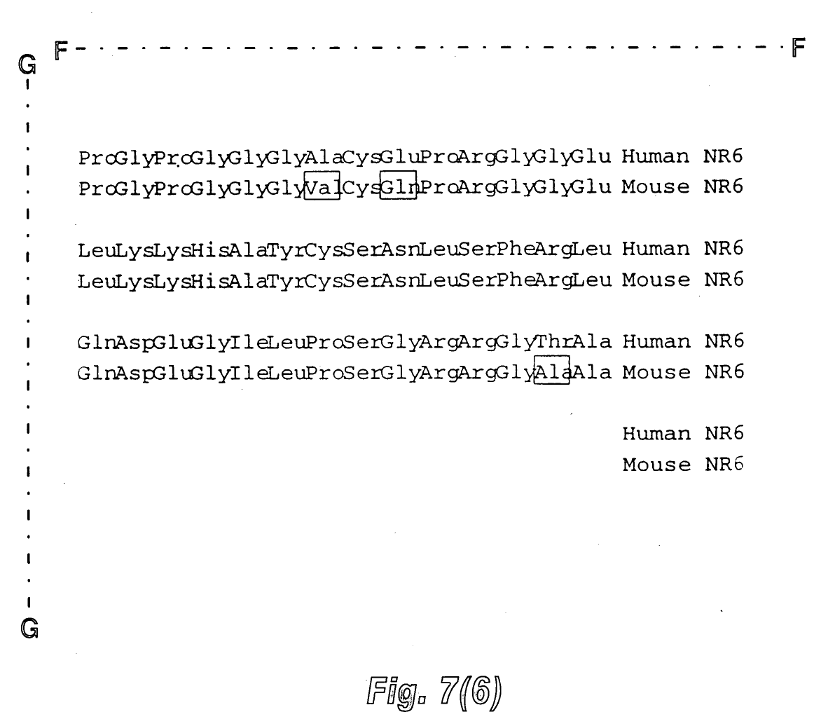

The huNR6 probe was excised from pCR-Blunt using EcoRI, 3′ end labelled with 32P (Pharmacia Biotech Ready To Go DNA Labelling Beads Cat # 27-9240-01) and used to screen the placental cDNA library (standard methods, duplicate filters, 106 plaques screened, high stringency washes—0.2×SSC, 0.1% SDS, 65° C.). Twenty positives were identified on primary screening and following two rounds of plaque purification, eighteen cloned tertiary phage stocks containing inserts ranging from ˜1-3 kb in size remained. Phage clone #11 was selected for thorough sequencing and found to contain 2079 bp insert, with an ORF of 1260 bases, 515 bp of 5′UTR and 304 bp of 3′UTR. The sequence of the ORF and the corresponding amino acid translation showed a high degree of homology to the corresponding mouse NR6 cDNA and amino acid sequences (88% and 95% respectively, FIGS. 6 and 7).

### EXAMPLE 18

### Human NR6 Expression Vectors

pEF-N′-FLAG/hNR6

The coding region of the mature human NR6 protein was amplified using PCR to introduce in frame Asc I restriction enzyme sites at the 5′ and 3′ ends using the following oligonucleotides:

[SEQ ID NO: 47]

| | |
|---|---|
| 5′Oligo | 5′-TCAGGCGCGCCTTGCCCACACAGCTGTGATC-3′ |

[SEQ ID NO: 48]

| | |
|---|---|
| 3′Oligo | 5′-TCAGGGCGCGCCTTATCTGGCAGGACCTCT-3′ |

The resulting PCR derived DNA fragment was then digested with Asc I and cloned into the Mlu I site of pEF-FLAG-S. Expression of NR6 is under control of the polypeptide elongation factor 1a promoter and results in the secretion, using the IL3 signal sequence from pEF-FLAG, of N-terminal FLAG-tagged NR6 protein.

pEF-C′-FLAG/hNR6

The complete coding region, including the endogenous signal sequence, of human NR6 protein was amplified using PCR to introduce in frame Asc I restriction enzyme sites at the 5′ and 3′ ends using the following oligonucleotides:

[SEQ ID NO: 49]

| | |
|---|---|
| 5′Oligo | 5′-TCAGGCGCGCCTGCCCGCCGGCCGC-3′ |

[SEQ ID NO: 50]

| | |
|---|---|
| 3′Oligo | 5′-ATAAGGCGCGCCCTGGCAGGACCTCTCG-3′ |

The resulting PCR derived DNA fragment was then digested with Asc I and cloned into the Asc I site of pEF-FLAG-I. Expression of NR6 is under control of the polypeptide elongation factor 1a promoter and results in the secretion, using the endogenous NR6 signal sequence, of C-terminal FLAG-tagged NR6 protein.

pEF-N′-I-SPY/hNR6

The coding region of the mature human NR6 protein was amplified using PCR to introduce in frame Asc I restriction enzyme sites at the 5′ and 3′ ends using the following oligonucleotides:

[SEQ ID NO: 51]

| | |
|---|---|
| 5′Oligo | 5′-TCAGGCGCGCCTTGCCCACACAGCTGTGATC-3′ |

[SEQ ID NO: 52]

| | |
|---|---|
| 3′Oligo | 5′-TCAGGGCGCGCCTTATCTGGCAGGACCTCT-3′ |

The resulting PCR derived DNA fragment was then digested with Asc I and cloned into the Mlu I site of pEF-1-SPY-S. In this vector the region encoding the FLAG tag has been excised from pEF-FLAG-S and replaced with sequence encoding an I-SPY epitope tag (QYPALT, AMRAD Biotech, Australia). Expression of NR6 is under control of the polypeptide elongation factor 1a promoter and results in the secretion, using the IL3 signal sequence from pEF-FLAG, of N-terminal FLAG-tagged NR6 protein.

pEF-C′-I-SPY/hNR6

The complete coding region, including the endogenous signal sequence, of human NR6 protein was amplified using PCR to introduce in frame Asc I restriction enzyme sites at the 5′ and 3′ ends using the following oligonucleotides:

[SEQ ID NO: 53]

| | |
|---|---|
| 5′Oligo | 5′-TCAGGCGCGCCTGCCCGCCGGCCGC-3′ |

[SEQ ID NO: 54]

| | |
|---|---|
| 3′Oligo | 5′-ATAAGGCGCGCCCTGGCAGGACCTCTCG-3′ |

The resulting PCR derived DNA fragment was then digested with Asc I and cloned into the Asc I site of pEF-1-SPY-I (see above for details). Expression of NR6 is under control of the polypeptide elongation factor 1a promoter and results in the secretion, using the endogenous NR6 signal sequence, of C-terminal FLAG-tagged NR6 protein.

### EXAMPLE 19

### Expression, Purification and Characterisation of CHO Human NR6

A. Transient Expression and Analysis of NR6

Transient Expression of C′-Terminal FLAG-Tagged Human NR6

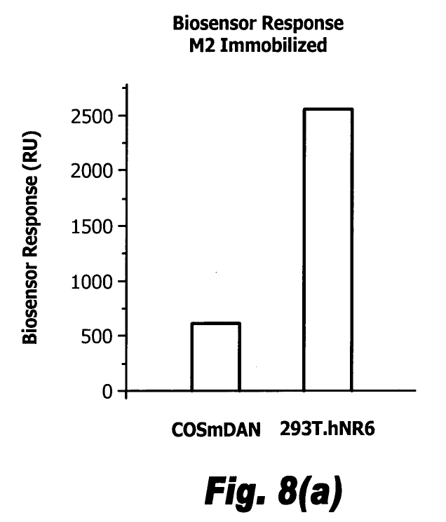

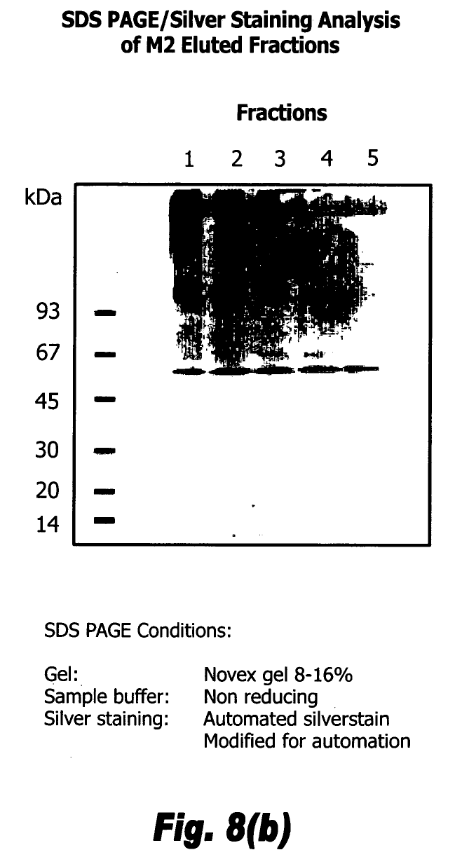

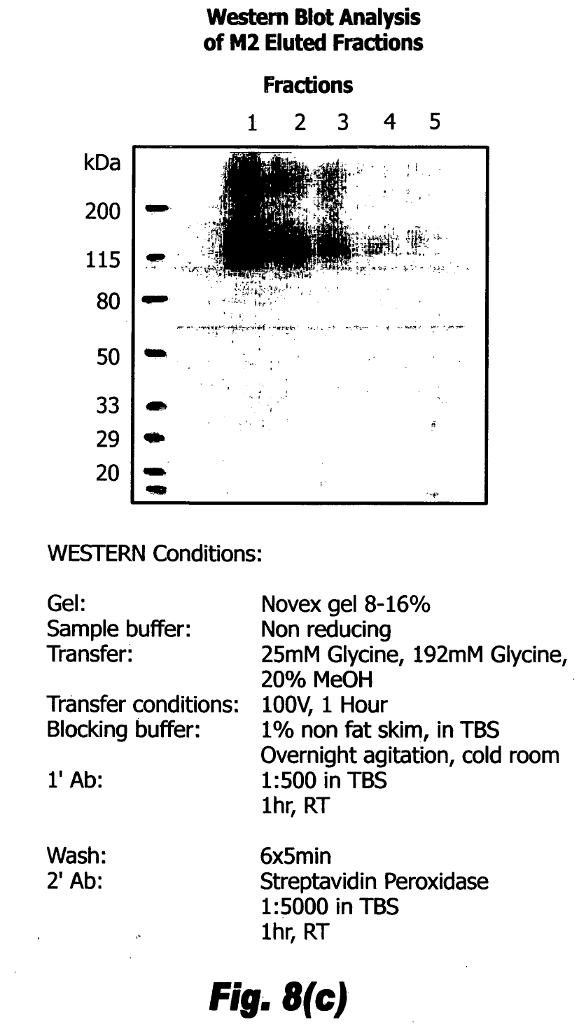

For transient expression of human NR6 the pEF-C′-FLAG/hNR6 expression construct described above was transfected into 293T cells using Lipofectamine (Gibco BRL, USA) according to the manufacturers instructions. Briefly, cells grown to approximately 70-80% confluence in 75 cm2 tissue culture flasks were washed in serum free DMEM media then exposed to a mixture of pEF-C′-FLAG/hNR6 and Lipofectamine diluted in DMEM. After 5 hours at 37° C. with 5% CO2 the cells were washed once with DMEM and incubated for a further 16 hours in DMEM supplemented with 10% v/v FCS, glutamine and antibiotics (DM10). At this time the DM10 was removed and replaced with a further 10 ml/flask of fresh DM10 and transfected cells incubated for a further 48 hours. Supernatants containing secreted human NR6 were recovered, centrifuged and filtered to remove cell debri, then stored at 4° C. Expression and purification was monitored by Biosensor analysis (BiaCore™, Sweden) where anti-FLAG peptide monoclonal antibody (M2, Kodak Eastman, USA) was bound to the sensorchip. Where multiple fractions were analysed for binding to the sensor surface (resonance units) the chip was desorbed with 50 mM Diethylamine pH 12.0 prior to application of the next sample. Biosensor analysis indicated that the transfected 293T cells secreted significant quantities of FLAG-tagged human NR6 protein into the surrounding media (FIG. 8A). The conditioned media (5 ml) was applied to M2 affinity resin (1 ml) under gravity. To enhance recovery the unbound fraction was reapplied to the column 4 times prior to extensive washing of the column with 200 volumes of Buffer A (see Example 14).

The bound fraction was eluted from the column with 10×1 ml volumes of 100 mg/ml FLAG peptide (Kodak Eastman) in Tris-buffered saline. The first 5 fractions were electrophoresed on an SDS-PAGE gel under non-reducing conditions. Silver staining revealed a band of the expected size for dimeric NR6 at approximately 120 kDa in fractions 1-3 (FIG. 8B). To confirm that this band was indeed NR6, an identical gel was subjected to Western blot analysis using the M2 monoclonal antibody. Fractions were electrophoresed under non-reducing conditions, transferred to a PVDF membrane then probed with a biotinylated M2 antibody. Bound antibody was detected using a Streptavidin-HRPO conjugate and ECL substrate. Subsequent autoradiography indicated a band of the expected size for dimeric NR6 at approximately 120 kDa (FIG. 8C).

N-Terminal Amino Acid Sequence of C-Terminal FLAG-Tagged NR6

For determination of the N-terminal amino acid sequence, C-terminal FLAG-tagged NR6 was purified from 75 ml of transfected 293T cell supernatant by M2 affinity chromatography as described above. Peak fractions (as determined by SDS-PAGE) were concentrated by lyophillization, resuspended in 0.5 ml and applied to a Superose 12 size exclusion column (Pharmacia, Flow rate 0.5 ml/min, 1 min fractions in 1% w/v ammonium bicarbonate, pH7.8). Peak fractions containing NR6, as determined by Biosensor and SDS-PAGE analysis, were sunjected to N-terminal sequence analysis using a Hewlett Packard sequencer with the indicated N-terminus at Ala40. This is identical to the N-terminus of mature CHO cell derived murine NR6.

NR6 is Secreted as a Homodimer

Western blot analysis following non-reducing and reducing SDS-PAGE and N′-terminal sequence analysis indicated that the secreted form of NR6 was as a homodimer rather than a heterodimer. To further confirm secretion of homodimeric NR6, 293 T cells were transiently cotransfected (Lipofectamine, as above) with vectors encoding C′-terminal FLAG-tagged NR6 and C′-terminal I-SPY-tagged NR6. For control purposes 293T cells were also transfected with each vector alone.

Supernatants from each transfection were immunoprecipitated with resin coupled—monoclonal antibody specific for either I-SPY or FLAG epitopes. The precipitates were then electrophoresed on SDS-PAGE, transferred to PVDF and probed with anti-FLAG antibody according to the standard protocol. FLAG specific reactivity of the appropriate molecular weight was detected in appropriate controls and in supernatants from cotransfections following precipitation with both anti-FLAG and anti-I-SPY coupled resins (results not shown). This indicates that FLAG-tagged and I-SPY tagged monomers are associating to form homodimers.

B. Production of Stable Cell Lines Secreting Dimeric Human NR6

For the generation of stable cell lines expressing human NR6, CHO cells and 293T cells were cotransfected with the pEF-C′-FLAG/hNR6 or pEF-N′-FLAG/hNR6 expression constructs and a vector incorporating a gene encoding puromycin resistance using Lipofectamine (Gibco BRL, USA) according to the manufacturers instructions. Following selection in puromycin (25 mg/ml, Sigma) resistant cells were cloned in 96 well microtitre plates by limiting dilution and clones assayed for NR6 production by a combination of Dot-blot analysis and Biosensor analysis (as above). For Dot-blot analysis 50 ml of supernatant from each clone was transferred to nitrocellulose membrane using a Dot-blot apparatus (BioRad, USA). The nitrocellulose was then incubated in blocking buffer (Phosphate buffered saline, PBS+1% Casein) for 30 min, washed in PBS and then probed with anti-FLAG M2 antibody (1:1000 in blocking buffer, 60 min), washed again and bound M2 detected using a HRPO conjugated anti-mouse antibody (Silenus, 1:2000 in blocking buffer, 60 min) used in conjunction with TMB substrate (Boehringer Mannheim).

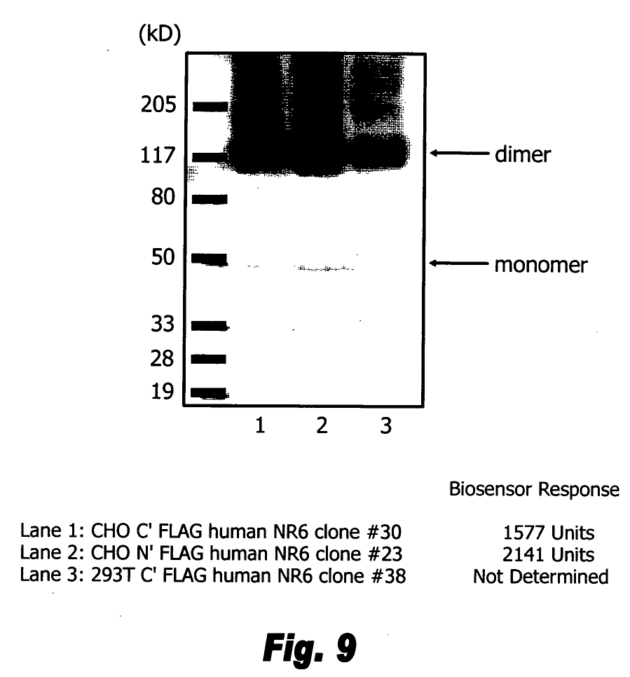

Following Dot-blot and Biosensor analysis 6 CHO cell clones expressing C-terminal FLAG-tagged human NR6, 6 CHO cell clones expressing N-terminal FLAG-tagged human NR6, and 6 293T cell clones expressing C-terminal FLAG-tagged human NR6 were selected and expanded for further analysis. Following further analysis a single clone was selected from each group of 6 for expansion and production of human NR6 for subsequent biological analysis. Biosensor analysis of supernatant from each of these clones indicated relatively high level production of NR6 (FIG. 9) and Western blot analysis confirmed that the dominant form of FLAG-tagged protein was a dimer of molecular weight approx. 120 kDa (FIG. 9).

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

1. Du, X. X. and Williams, D. A. (1994) Blood 83: 2023-2030.
2. Yang, Y. C. and Yin, T. (1992) Biofactors 4: 15-21.
3. Paul, S. R., Bennett, F., Calvetti, J. A., Kelleher, K., Wood, C. R., O'Hara, R. J. J., Leary, A. C., Sibley, B., Clark, S. C., Williams, D. A. and Yang, Y.-C. (1990) Proc. Natl. Acad. Sci. USA 87: 7512.
4. Musashi, M., Clark, S. C., Sudo, T., Urdal, D. L., and Ogawa, M. (1991) Blood 78: 1448-1451.
5. Schibler, K. R., Yang, Y. C. and Christensen, R. D. (1992) Blood 80: 900-3.
6. Tsuji, K., Lyman, S. D., Sudo, T., Clark, S. C., and Ogawa, M. (1992) Blood 79: 2855-60.
7. Burstein, S. A., Mei, R. L., Henthorn, J., Friese, P. and turner, K. (1992) J. Cell. Physiol. 153: 305-12.
8. Hangoc, G., Yin, T., Cooper, S., Schendel, P., Yang, Y. C. and Broxmeyer, H. E. (1993) Blood 81: 965-72.
9. Teramura, M., Kobayashi, S., Hoshino, S., Oshimi, K. and Mizoguchi, H. (1992) Blood 79: 327-31.
10. Yonemura, Y., Kawakita, M., Masuda, T., Fujimoto, K., Kato, K. and Takatsuki, K. (1992) Exp. Hematol. 20: 1011-6.
11. Baumann, H. and Schendel, P. (1991) J. Biol. Chem. 266: 20424-7.
12. Kawashima, I., Ohsumi, J., Mita-Honjo, K., Shimoda-Takano, K., Ishikawa, H., Sakakibara, S., Miyadai, K. and Takiguchi, Y. (1991) Febs. Lett. 283: 199-202.
13. Keller, D. C., Du, X. X., Srour, E. f., Hoffman, R. and Williams, D. A. (1993) Blood 82: 1428-35.
14. Sambrook et al (1989) Cloning: A Laboratory Manual. Cold Spring Harbour Laboratory, Cold Spring Harbour, N.Y.
15. Chirgwin et al (1979) Biochemistry 18: 5294-5299.
16. Mizushima and Nagata (1990) Nucl. Acids Res., 18: 5322.
17. FEBS Lett (1994) 356: 244-248.
18. Bazan, J. F. (1990) Proc Natl Acad Sci USA, 87, 6934-8
19. de Vos, A. M., Ultsch, M. and Kossiakoff, A. A. (1992) Science, 255, 306-12
20. Layton, M. J., Cross, B. A., Metcalf, D., Ward, L. D., Simpson, R. J. and Nicola, N. A. (1992) Proceedings of the National Academy of Sciences of the United States of America 89: 8616-8620
21. Taga, T., Hibi, M., Hirata, T., Tamasaki, K., Tasukawa, K., Matsuda, T., Hirano, T. and Kishimoto, T. (1989) Cell 58: 573-581
22. Merberg, D. M., Wolf, S. F. and Clark, S. C. (1992) Sequence similarity between NKSF and the IL-6/G-CSF family (1992) Immunology Today 13: 77-78
23. Cearing, D. P. and Cosman, D. (1991) Cell 66:9-10
24. Wrighton, N. C., Farrell, F. X., Chang, R., Kashyap, A. K., Barbone, F. P., Mulcahy, L. S., Johnson, D. L., Barrett, R. W., Jolliffe, L. K. and Dower, W. J. (1996) Science 273: 458464.
25. Cwirla, S. E., Balasubramanian, P., Duffin, D. J., Wagstrom, C. R., Gates, C. M., Singer, S. C., Davis, A. M., Tansik, R. L., Mattheakis, L. C., Boytos, C. M., Schatz, P. J., Baccanari, D. P., Wrighton, N. C., Barret, R. W. and Dower, W. J. (1997) Science 276: 1696-9, 1997
26. Samuel Davis et al (1996) Cell 87:1161-1169.
27. Chitra Suri et al (1996) Cell 87: 1171-1180.
28. Nicholas C. Wrighton et al (1996) Science 273: 458-463.
29. Oded Livnah et al (1996) Science 273: 464-471.
30. Cwirla, Steven E. et al (1997) Science 276: 1696-1699.

1. A nucleic acid molecule comprising a sequence of nucleotides encoding or complementary to a sequence encoding a novel haemopoietin receptor or derivative thereof having the motif:

| | |
|---|---|
| Trp Ser Xaa Trp Ser, | [SEQ ID NO: 1] |

wherein Xaa is any amino acid.

2. A nucleic acid molecule according to claim 1 wherein Xaa is Asp or Glu.

3. A nucleic acid molecule according to claim 1 or 2 wherein said nucleic acid molecule is capable of hybridisation under low stringency conditions at 42° C. to:

| | |
|---|---|
| 5′ (A/G)CTCCA(A/G)TC(A/G)CTCCA 3′; | [SEQ ID NO: 7] |
| and | |
| 5′ (A/G)CTCCA(C/T)TC(A/G)CTCCA 3′. | [SEQ ID NO: 8] |

4. A nucleic acid molecule according to claim 3 comprising a sequence of nucleotides substantially as set forth in SEQ ID NO:12 or a nucleotide sequence having at least 60% similarity to the nucleotide sequence set forth in SEQ ID NO:12 or a nucleotide sequence capable of hybridising thereto under low stringency conditions at 42° C.

5. A nucleic acid molecule according to claim 3 comprising a sequence of nucleotides substantially as set forth in SEQ ID NO:14 or a nucleotide sequence having at least 60% similarity to the nucleotide sequence set forth in SEQ ID NO:14 or a nucleotide sequence capable of hybridising thereto under low stringency conditions at 42° C.

6. A nucleic acid molecule according to claim 3 comprising a sequence of nucleotides substantially as set forth in SEQ ID NO:16 or a nucleotide sequence having at least 60% similarity to the nucleotide sequence set forth in SEQ ID NO:16 or a nucleotide sequence capable of hybridising thereto under low stringency conditions at 42° C.

7. A nucleic acid molecule according to claim 3 comprising a sequence of nucleotides substantially as set forth in SEQ ID NO:18 or 24 or a nucleotide sequence having at least 60% similarity to the nucleotide sequence set forth in SEQ ID NO:18 or 24 or a nucleotide sequence capable of hybridising thereto under low stringency conditions at 42° C.

8. A nucleic acid molecule according to claim 3 comprising a sequence of nucleotides substantially as set forth in SEQ ID NO:28 or a nucleotide sequence having at least 60% similarity to the nucleotide sequence set forth in SEQ ID NO:28 or a nucleotide sequence capable of hybridising thereto under low stringency conditions at 42° C.

9. A nucleic acid molecule according to claim 3 comprising a sequence of nucleotides substantially as set forth in SEQ ID NO:38 or a nucleotide sequence having at least 60% similarity to the nucleotide sequence set forth in SEQ ID NO:38 or a nucleotide sequence capable of hybridising thereto under low stringency conditions at 42° C.

10. A nucleic acid molecule according to claim 4 or 5 or 6 or 7 or 8 or 9 wherein said haemopoietin receptor is of murine origin.

11. A nucleic acid molecule according to claim 9 wherein said haemopoietin receptor is of human origin.

12. An expression vector comprising a nucleic acid molecule selected from the list consisting of:
(i) a nucleotide sequence as set forth in SEQ ID NO: 12;
(ii) a nucleotide sequence as set forth in SEQ ID NO:14;
(iii) a nucleotide sequence as set forth in SEQ ID NO: 16;
(iv) a nucleotide sequence as set forth in SEQ ID NO: 18;
(v) a nucleotide sequence as set forth in SEQ ID NO:24;
(vi) a nucleotide sequence as set forth in SEQ ID NO:28; and
(vii) a nucleotide sequence as set forth in SEQ ID NO:38.

13. A method for cloning a nucleotide sequence encoding a haemopoietin receptor having the characteristics of NR6 or a derivative thereof, said method comprising searching a nucleotide database for a sequence which encodes an amino acid sequence as set forth in one or more of SEQ ID NO:1, SEQ ID NO:7 and/or SEQ ID NO:8, designing one or more oligonucleotide primers based on the nucleotide sequence located in said search, screening a nucleic acid library with said one or more oligonucleotides and obtaining a clone therefore which encodes NR6 or a part or derivative thereof.

14. An isolated nucleic acid molecule comprising a sequence of nucleotides encoding a haemopoietin receptor or derivative thereof having an amino acid sequence substantially as set forth in SEQ ID NO:13 or having at least about 50% similarity thereto.

15. An isolated nucleic acid molecule comprising a sequence of nucleotides encoding a haemopoietin receptor or derivative thereof having an amino acid sequence substantially as set forth in SEQ ID NO:15 or having at least about 50% similarity thereto.

16. An isolated nucleic acid molecule comprising a sequence of nucleotides encoding a haemopoietin receptor or derivative thereof having an amino acid sequence substantially as set forth in SEQ ID NO:17 or having at least about 50% similarity thereto.

17. An isolated nucleic acid molecule comprising a sequence of nucleotides encoding a haemopoietin receptor or derivative thereof having an amino acid sequence substantially as set forth in SEQ ID NO:19 or having at least about 50% similarity thereto.

18. An isolated nucleic acid molecule comprising a sequence of nucleotides encoding a haemopoietin receptor or derivative thereof having an amino acid sequence substantially as set forth in SEQ ID NO:25 or having at least about 50% similarity thereto.

19. An isolated nucleic acid molecule comprising a sequence of nucleotides encoding a haemopoietin receptor or derivative thereof having an amino acid sequence substantially as set forth in SEQ ID NO:29 or having at least about 50% similarity thereto.

20. An isolated novel haemopoietin receptor comprising the amino acid motif:

| | |
|---|---|
| Trp Ser Xaa Trp Ser | [SEQ ID NO: 1] |

wherein Xaa is any amino acid.

21. An isolated haemopoietin receptor according to claim 20 wherein Xaa is Asp or Glu.

22. An isolated haemopoietin receptor according to claim 21 comprising the amino acid sequence substantially as set forth in SEQ ID NO: 13.

23. An isolated haemopoietin receptor according to claim 21 comprising the amino acid sequence substantially as set forth in SEQ ID NO: 15.

24. An isolated haemopoietin receptor according to claim 21 comprising the amino acid sequence substantially as set forth in SEQ ID NO: 17.

25. An isolated haemopoietin receptor according to claim 21 comprising the amino acid sequence substantially as set forth in SEQ ID NO: 19.

26. An isolated haemopoietin receptor according to claim 21 comprising the amino acid sequence substantially as set forth in SEQ ID NO:25.

27. An isolated haemopoietin receptor according to claim 21 comprising the amino acid sequence substantially as set forth in SEQ ID NO:29.

28. A method for modulating expression of NR6 in a mammal, said method comprising contacting a genetic sequence encoding said NR6 with an effective amount of a modulator of NR6 expression for a time and under conditions sufficient to up-regulate or down-regulate or otherwise modulate expression of NR6, wherein the genetic sequence encoding said NR6 is selected from the nucleotide sequence set forth in SEQ ID NO:12 or 14 or 16 or 18 or 24 or 28 or 38 or is a sequence having at least about 60% similarity to at least one of SEQ ID NO:12 or 14 or 16 or 18 or 24 or 28 or 38 and is capable of hybridising thereto under low stringency conditions at 42° C.

29. A method of modulating activity of NR61n a mammal, said method comprising administering to said mammal, a modulating effective amount of a molecule for a time and under conditions sufficient to increase or decrease NR6 activity wherein said NR6 comprises an amino acid sequence:
(i) encoded by a nucleotide sequence selected from the nucleotide sequence set forth in SEQ ID NO:12 or 14 or 16 or 18 or 24 or 28 or 38 or a nucleotide sequence having at least 60% similarity to the nucleotide sequence set forth in SEQ ID NO:12 or 14 or 16 or 18 or 24 or 28 or 38 and which is capable of hybridising thereto under low stringency conditions at 42° C.; and
(ii) substantially as set forth in SEQ ID NO:12 or 14 or 16 or 18 or 32 or 30 or a sequence having at least 50% similarity thereto.

30. A pharmaceutical composition comprising an NR6 receptor in soluble form and one or more pharmaceutically acceptable carriers and/or diluents wherein said NR6 comprises the amino acid sequence:
(i) encoded by a nucleotide sequence selected from the nucleotide sequence set forth in SEQ ID NO:12 or 14 or 16 or 18 or 24 or 28 or 38 or a nucleotide sequence having at least 60% similarity to the nucleotide sequence set forth in SEQ ID NO:12 or 14 or 16 or 18 or 24 or 28 or 38 and which is capable of hybridising thereto under low stringency conditions at 42° C.; and
(ii) substantially as set forth in SEQ ID NO:12 or 14 or 16 or 18 or 32 or 30 or a sequence having at least 50% similarity thereto.

31. An isolated antibody or a preparation of antibodies to an NR6 receptor, said NR6 receptor comprising the amino acid sequence:
(i) encoded by a nucleotide sequence selected from the nucleotide sequence set forth in SEQ ID NO:12 or 14 or 16 or 18 or 24 or 28 or 38 or a nucleotide sequence having at least 60% similarity to the nucleotide sequence set forth in SEQ ID NO:12 or 14 or 16 or 18 or 24 or 28 or 38 and which is capable of hybridising thereto under low stringency conditions at 42° C.; and
(ii) substantially as set forth in SEQ ID NO:12 or 14 or 16 or 18 or 24 or 28 or 38 or a sequence having at least 50% similarity thereto.

32. A transgenic animal comprising a mutation in at least one allele of the gene encoding NR6.

33. A transgenic animal according to claim 32 comprising a mutation in two alleles of the gene encoding NR6.

34. A transgenic animal according to claim 32 or 33 wherein said animal is a murine animal.
